SEQUENCE LISTING (454 sequences)

SEQ ID NO: 1 - DNA, 339 bp, Homo sapiens
ctcgaggagtcagggggaggcttggtacagcctggggggtccctgagactctcctgtgaagcctctggattcacctttagcagctatgccatgagctgggtccgccaggctccagggaaggggctggagtgggtctcagttattagtggtaatggtggtagcacatactacgcagactccgtgaagggccggttcaccttctccagagacaattccaagaacacgctgtatctgcgaatgaacagcctgagagccgaggacacggccgtatattactgtgcgaaagatagattaagtcagtgggagttactacagattgactactggggccagggaacc

SEQ ID NO: 2 - protein, 113 aa, Homo sapiens
LEESGGGLVQPGGSLRLSCEASGFTFSSYAMSWVRQAPGKGLEWVSVISGNGGSTYYADSVKGRFTFSRDNSKNTLYLRMNSLRAEDTAVYYCAKDRLSQWELLQIDYWGQGT

SEQ ID NO: 3 - DNA, 354 bp, Homo sapiens
ctcgaggagtctgggggaggcttggtaaagccgggggggtcccttagactctcctgcgcaggctctggtttcactttcagtaacgtctggatgaactgggtccgccaggctccagggaaggggctggaatgggtcggccgtattaaaatcaagactgatggtgggacaacagactacgctacaccggaatgggtcggccgtattaaaatcaagactgatggtgggacaacagactacgctacaccgaacagcctgaaaaccgaggacacagccgtatattactgtaccacagatgattggtataacactagaggctactactactacggtatggacgtctggggccaagggacc

SEQ ID NO: 4 - protein, 118 aa, Homo sapiens
LEESGGGLVKPGGSLRLSCAGSGFTFSNVWMNWVRQAPGKGLEWVGRIKIKTDGGTTDYATPEWVGRIKIKTDGGTTDYATPNSLKTEDTAVYYCTTDDWYNTRGYYYYGMDVWGQGT

SEQ ID NO: 5 - DNA, 338 bp, Homo sapiens
ctcgaggagtctgggggaggcttggtccagcctggggggtccctgaaactctcctgtgcagcctctgggttcgccttcagtggctctgctctgcactgggtccgccaggcttccgggagagggctggagtgggttggccgtattagaaccaaagctaacaattacgcgacagtgtatggtgcgtcggtgaagggcaggttcaccatctccaaagacaatgccaagaactcactgtatctgcaaatgaacagcctgagagccgaggacacggctgtgtattactgtgcgagactgcttggcactggctggtacggagttgactactggggccagggaac

SEQ ID NO: 6 - protein, 113 aa, Homo sapiens
LEESGGGLVQPGGSLKLSCAASGFAFSGSALHWVRQASGRGLEWVGRIRTKANNYATVYGASVKGRFTISKDNAKNSLYLQMNSLRAEDTAVYYCARLLGTGWYGVDYWGQGT

SEQ ID NO: 7 - DNA, 333 bp, Homo sapiens
ctcgagtcggggggaggcttggtacagcctggggggtccctgagactctcctgtgcagcctctggattcacctttagcagctatgccgtgagctgggtccgccaggctccagggaaggggctggagtgggtctcagctattagtggtagtggtggtagcacatattacgcagacttagtgaagggccggttcgccatctccagagacaattccaagaacacgctgtatctgcaaatgaacagcctgagagccgaggacacggccgtatattactatgcgaaagatcaaatgaacttaccgtacaactggttcgacccctggggccagggaacc

SEQ ID NO: 8 - protein, 111 aa, Homo sapiens
LESGGGLVQPGGSLRLSCAASGFTFSSYAVSWVRQAPGKGLEWVSAISGSGGSTYYADLVKGRFAISRDNSKNTLYLQMNSLRAEDTAVYYYAKDQMNLPYNWFDPWGQGT

SEQ ID NO: 9 - DNA, 345 bp, Homo sapiens
ctcgagcagtctgggggagacttggtacagcctggggggtccctgagactctcctgtgtagcctctggattcattttcagtaattatgacatgcactgggtccgccaacctgcaggaaaaggtctggagtgggtcgcaaccattggtactgctactgacacatactatccaggctccgtgaagggccgattcaccatctccagagataatgccaagagctccttctttcttcgaatgaacagcctgggagccgaggacacggctgtttattactgtgcaaaaggaggaggagaccagaggactacggcgactacgcggtacttcgatctgtggggacgtggcacc

SEQ ID NO: 10 - protein, 115 aa, Homo sapiens
LEQSGGDLVQPGGSLRLSCVASGFIFSNYDMHWVRQPAGKGLEWVATIGTATDTYYPGSVKGRFTISRDNAKSSFFLRMNSLGAEDTAVYYCAKGGGDQRTTATTRYFDLWGRGT

SEQ ID NO: 11 - DNA, 339 bp, Homo sapiens
ctcgaggagtctggagcagaagtgaagaagccgggcgaaaatcttaagatctcctgcgaggcttctggatacaattttgtcaatcactggatcggctgggtgcgccagatgcccgggagaggccttgagtggatgggccgcatctatcctggagactctgaaaccagattcagtccgtccttccaagggcaggtcaccatctcagtcgacaaaactctgagtaccgcctccctacagtggaacagtctcaagacgtcggacagcgccacatattattgtgtgacactggggcgctggagcagctggcaaggtggggcgctctcatggggccagggaacc

SEQ ID NO: 12 - protein, 113 aa, Homo sapiens
LEESGAEVKKPGENLKISCEASGYNFVNHWIGWVRQMPGRGLEWMGRIYPGDSETRFSPSFQGQVTISVDKTLSTASLQWNSLKTSDSATYYCVTLGRWSSWQGGALSWGQGT

SEQ ID NO: 13 - DNA, 342 bp, Homo sapiens
ctcgaggagtctggggctgaagtgaagaaacctggggcctcagtggaggtctcctgcaagacctctggatacaccttcatcgagtaccctatacactgggtgcgacaggcccctggacaagggcttgagtggacgggctggatcacccctatcgatggtggcacagactttgcagggaagtttcagggccgggccaccatgaccagcgacatgtccaccagcacagccaagttggtcctgaagagcctgaggtctgacgacacggccgtctatttctgtgcgcgggcacggggggggggatttttggacaggttattgtactcggactggggccagggaacc

SEQ ID NO: 14 - protein, 114 aa, Homo sapiens
LEESGAEVKKPGASVEVSCKTSGYTFIEYPIHWVRQAPGQGLEWTGWITPIDGGTDFAGKFQGRATMTSDMSTSTAKLVLKSLRSDDTAVYFCARARGGGFLDRLLYSDWGQGT

SEQ ID NO: 15 - DNA, 309 bp, Homo sapiens
ctcgaggagtctgggggaggcttggtcaagcctggagggtccctgaggctctcctgtgcagcctctggattcaccttcagtgactactacatgagttggatccgccaggctccagggaaggggctggaatttatatcatacattagcagtggtggtgacaccatacaccacgcagactctgtgaagggccgattcaccatctccagggacaacgccaagaagtcactgtatctccaaatgaacagcctgagagtcgaggatacggccgtatattactgtgcgtgccgtggggtctggggccagggaacc

SEQ ID NO: 16 - protein, 103 aa, Homo sapiens
LEESGGGLVKPGGSLRLSCAASGFTFSDYYMSWIRQAPGKGLEFISYISSGGDTIHHADSVKGRFTISRDNAKKSLYLQMNSLRVEDTAVYYCACRGVWGQGT

SEQ ID NO: 17 - DNA, 309 bp, Homo sapiens
ctcgaggagtcatggggaggcttggtcaagcctggagggtccctgaggctctcctgtgcagcctctggattcaccttcagtgactactacatgagttggatccgccaggctccagggaaggggctggaatttatatcatacattagtagtggtggtgacaccatacaccacgcagactctgtgaagggccgattcaccatctccagggacaacgcccagaagtcactgtatctccaaatgaacagcctgagagtcgaggacacggccgtatattactgtgcgtgccgtggggtctggggccagggaacc

SEQ ID NO: 18 - protein, 102 aa, Homo sapiens
LEESWGGLVKPGGSLRLSCAASGFTFSDYYMSWIRQAPGKGLEFISYISSGGDTIHHADSVKGRFTISRDNAQKSLYLQMNSLRVEDTAVYYCACRGVWGQG

SEQ ID NO: 19 - DNA, 345 bp, Homo sapiens
ctcgagtcggggggaggcttggtccagcctggggggtccctgaaactctcctgtgcagcctctgggttcgccttcagtggctctgctctgcactgggtccgccaggcttccgggagagggctggagtgggttggccgtattagaaccaaagctaacaattacgcgacagtgtatggtgcgtcggtgaagggcaggttcaccatctccagagatgattcaaagagcacggcgtatctgctaatgaacagcctgaaaaccgaggacacggccgtctattactgtactagttatgataccagctatgataggagtggttattatttgaactactggggccagggaacc

SEQ ID NO: 20 - protein, 115 aa, Homo sapiens
LESGGGLVQPGGSLKLSCAASGFAFSGSALHWVRQASGRGLEWVGRIRTKANNYATVYGASVKGRFTISRDDSKSTAYLLMNSLKTEDTAVYYCTSYDTSYDRSGYYLNYWGQGT

SEQ ID NO: 21 - DNA, 348 bp, Homo sapiens
ctcgagtctgggggaggcttgggacagcctggggggtccctgagactctcctgtgcagcctctggatttacctttagcagctatgccatgagctgggtccgccaggctccagggaaggggctggagtgggtctcagctattagtgataggggggagagcacatactacgcagactccgtgaagggccggttcaccatctccagggacaattctaagaacacgctgtatgtgcaaatgaacagcctgagagccgaggacacggccctatatttctgcgcgaaagatcaatttctatggttcggggagtcaacagcgggtgatgcttttgatatctggggccaagggaca

SEQ ID NO: 22 - protein, 116 aa, Homo sapiens
LESGGGLGQPGGSLRLSCAASGFTFSSYAMSWVRQAPGKGLEWVSAISDRGESTYYADSVKGRFTISRDNSKNTLYVQMNSLRAEDTALYFCAKDQFLWFGESTAGDAFDIWGQGT

SEQ ID NO: 23 - DNA, 333 bp, Homo sapiens
ctcgagtcgggcccaggactggtgaagccttcacagaccctgtccctcacctgcactgtctctggtggctccatcagcagtggttactactggacctggatccgccagtacccagggaggggcctggagtggattggatacatctcttacagggggagcacctactacaacccgtccctcaagagtcgagttacaatatcactagacacgtctaagaaccagtttttcttgaacctgacctctgtgactgccgcggacacggccgtgtatttctgtgcgagagatcggagtagagcaacatctggtattcttgactactggggccagggaacc

SEQ ID NO: 24 - protein, 111 aa, Homo sapiens
LESGPGLVKPSQTLSLTCTVSGGSISSGYYWTWIRQYPGRGLEWIGYISYRGSTYYNPSLKSRVTISLDTSKNQFFLNLTSVTAADTAVYFCARDRSRATSGILDYWGQGT

SEQ ID NO: 25 - DNA, 339 bp, Homo sapiens
ctcgaggagtcagggggaggtgtggtacagccagggcggtccctgagactcccctgtacagcttctggattcatctttggtgattatgctatgagctgggtccgccaggctccagggaaggggctggagtggataggtttcgttagaagcaaagcttttggtgcgacaacacaatacgccgcatctgtgcaaggcagattcaccatctcaagagatgcttccaaaaatatcgcctatctgcaaatgaacagcctgaaaagcgaggacacagccatatattattgtactacttacggtagaaccacttggtactactttgactattggggccagggaacc

SEQ ID NO: 26 - protein, 113 aa, Homo sapiens
LEESGGGVVQPGRSLRLPCTASGFIFGDYAMSWVRQAPGKGLEWIGFVRSKAFGATTQYAASVQGRFTISRDASKNIAYLQMNSLKSEDTAIYYCTTYGRTTWYYFDYWGQGT

SEQ ID NO: 27 - DNA, 330 bp, Homo sapiens
ctcgagtcggggggaggcttggtcaagcctggagggtccctgcgactctcctgtttagcctctgggttcacctttagcagctatgccatgagctgggtccgccaggctccagggaaggggctggcgtgggtctcaactattagtggtagtggtgataacacatactacgcagactccgtgaagggccggttcaccatctccagagacaattccaagaacacggtgtatctgcaaatgaacagcctgagagccgaggacacggccgtctattactgtgcgaatcgtccggttcggggagtggactactttgactactggggccagggaacc

SEQ ID NO: 28 - protein, 109 aa, Homo sapiens
LESGGGLVKPGGSLRLSCLASGFTFSSYAMSWVRQAPGKGLAWVSTISGSGDNTYYADSVKGRFTISRDNSKNTVYLQMNSLRAEDTAVYYCANRPVRGVDYFDYWGQG

SEQ ID NO: 29 - DNA, 309 bp, Homo sapiens
ctcgaggagtctgggggaggcttggtcaagcctggagggtccctgaggctctcctgtgcagcctctggattcaccttcagtgactactacatgagttggatccgccaggctccagggaaggggctggaatttatatcatacattagtagtggtggtgacaccatacaccacgcagactctgtgaagggccgattcaccatctccagggacaacgccaagaagtcactgtatctccaaatgaacagcctgagagtcgaggacacggccgtatattactgtgcgtgccgtggggtctggggccagggaacc

SEQ ID NO: 30 - protein, 103 aa, Homo sapiens; identical to SEQ ID NO: 16

SEQ ID NO: 31 - DNA, 345 bp, Homo sapiens; identical to SEQ ID NO: 19

SEQ ID NO: 32 - protein, 115 aa, Homo sapiens; identical to SEQ ID NO: 20

SEQ ID NO: 33 - DNA, 351 bp, Homo sapiens
ctcgaggagtcaggggctgaggtgaagaagcctgggtcctcggtgaaggtctcctgcaaggcttctggaggcaccttcagcagttatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagatcgaagtggggttcttcgaagcagctcgccgatatggtacttcgatctctggggccgtggcacc

SEQ ID NO: 34 - protein, 117 aa, Homo sapiens
LEESGAEVKKPGSSVKVSCKASGGTFSSYAISWVRQAPGQGLEWMGGIIPIFGTANYAQKFQGRVTITADKSTSTAYMELSSLRSEDTAVYYCARDRSGVLRSSSPIWYFDLWGRGT

SEQ ID NO: 35 - DNA, 360 bp, Homo sapiens
ctcgaggagtcagggggaggcgtggtccagcctgggaggtccctgagactctcctgtgcagcgtctggattcagtttcagtaactatggcatgcactgggtccgccaggctccaggcaagggactggagtgggtggcagttatatggcatgatggaagtaataaagactatggcgactccgtgaagggccgattcagcatctccagagacaattccaggagaacgttgtatctgcaaatgaacaacttgagagccgaggacacggctatatactactgtgcgagagaggggggttaccgaaacgtcgcggatatattgcgccccccacctgatgcttttgataactggggccaggggaca

SEQ ID NO: 36 - protein, 120 aa, Homo sapiens
LEESGGGVVQPGRSLRLSCAASGFSFSNYGMHWVRQAPGKGLEWVAVIWHDGSNKDYGDSVKGRFSISRDNSRRTLYLQMNNLRAEDTAIYYCAREGGYRNVADILRPPPDAFDNWGQGT

SEQ ID NO: 37 - DNA, 333 bp, Homo sapiens
ctcgagtcggggggaggcttcgtacagcctggggggtctctgagactctcctgtgcagcctctggattcaccttcagggactatgccatgggctgggtccgccaggctccagggaaggggccggagtgggtctcaattattagtgctagtggtggttccatatactacgcagactccgtgaagggccgattcaccatctccagagacaacgccaagaacacactgtatctgcaaatgaacagtctcagagccgacgacacggctgtatactactgtgcaagacagaccagcagcagatggtatgattggttcgacccctggggccagggaacc

SEQ ID NO: 38 - protein, 111 aa, Homo sapiens
LESGGGFVQPGGSLRLSCAASGFTFRDYAMGWVRQAPGKGPEWVSIISASGGSIYYADSVKGRFTISRDNAKNTLYLQMNSLRADDTAVYYCARQTSSRWYDWFDPWGQGT

SEQ ID NO: 39 - DNA, 345 bp, Homo sapiens
ctcgaggagtctggggctgaggtgaagaagcctgggtcctcggtgaaggtctcctgcaaggcttctggagaccactatggtatcaactgggtgcgacaggcccctggacaagggcttgagtggatgggcggtatcatccctgtctttggcacaactacctacgcacagaagttccagggcagagccaccattaccgcggacgactccacggggacggcctttttggagctgaccagactgacatttgacgacacggccgtctatttctgtgcgacacctcaccaactgcatgtcctccggggcggtaaagccctctccccctgggactactggggccagggaacc

SEQ ID NO: 40 - protein, 115 aa, Homo sapiens
LEESGAEVKKPGSSVKVSCKASGDHYGINWVRQAPGQGLEWMGGIIPVFGTTTYAQKFQGRATITADDSTGTAFLELTRLTFDDTAVYFCATPHQLHVLRGGKALSPWDYWGQGT

SEQ ID NO: 41 - DNA, 348 bp, Homo sapiens
ctcgaggagtctggggctgaagtgaagaagccggggtcctcggtgaaggtctcctgcacggcttctggaggcatcttcagcaattatgctgtcatctgggtgcgacaggcccctggacaagggcttgaatggatgggagggttcatccccatgtttgatacagcaaaccacgcacagcacctccagggcagagtcacgatcaccgcgggcgattccacgagcgtcgtctatctggaactgcgcagcctgagatctgaagacaccgccatatatttttgcgcggcagccaaattgcaccctaactggaactttggaactttctactttgactcctggggccagggaacc

SEQ ID NO: 42 - protein, 116 aa, Homo sapiens
LEESGAEVKKPGSSVKVSCTASGGIFSNYAVIWVRQAPGQGLEWMGGFIPMFDTANHAQHLQGRVTITAGDSTSVVYLELRSLRSEDTAIYFCAAAKLHPNWNFGTFYFDSWGQGT

SEQ ID NO: 43 - DNA, 306 bp, Homo sapiens
ctcgagtctgggggaggcttggtcaagcctggagggtccctgaggctctcctgtgcagcctctggattcaccttcagtgactactacatgagttggatccgccaggctccagggaaggggctggaatttatatcatacattagtagtggtggtgacaccatacaccacgcagactctgtgaagggccgattcaccatctccagggacaacgccaagaagtcactgtatctccaaatgaacagcctgagagtcgaggacacggccgtatattactgtgcgtgccgtggggtctggggccagggaacc

SEQ ID NO: 44 - protein, 102 aa, Homo sapiens
LESGGGLVKPGGSLRLSCAASGFTFSDYYMSWIRQAPGKGLEFISYISSGGDTIHHADSVKGRFTISRDNAKKSLYLQMNSLRVEDTAVYYCACRGVWGQGT

SEQ ID NO: 45 - DNA, 333 bp, Homo sapiens
ctcgagtcggggggagacttggtacagcctggcgggtccctgagactctcctgtgtagcctctggattcacttttgatgattatgccatgcactgggtccggcagactccagggaagggcctggagtgggtctcaggtataagttggagaagtgattacagaggctatgcggactctgtgaagggccgattcaccatctccagagacaacgccaagaactccctgtatcttcaaatgaacagtctgggagttgaggacacggccttgtattactgtgcaaaaggcacgtattacgatattttgactggttattcttcctggggccagggaacc

SEQ ID NO: 46 - protein, 111 aa, Homo sapiens
LESGGDLVQPGGSLRLSCVASGFTFDDYAMHWVRQTPGKGLEWVSGISWRSDYRGYADSVKGRFTISRDNAKNSLYLQMNSLGVEDTALYYCAKGTYYDILTGYSSWGQGT

SEQ ID NO: 47 - DNA, 324 bp, Homo sapiens
ctcgagtctgggggaggcgtggtccagcctgggaggtccctgagactctcctgtgcagcgtctggattcaccctcaatagctatggcatgcactgggtccgccagactccaggcaaggggctggagtgggtggcaaacatatggaaggatggaattaataaatattatgcagactccgtgatgggccgagtcaccatctccagagacaattccaggaacacactgtatctccaaatgaacagcctgagagccgaggacacggctgtgtatttctgtgcgagagatttggactactctggtatggacgtctggggccagggaacc

SEQ ID NO: 48 - protein, 108 aa, Homo sapiens
LESGGGVVQPGRSLRLSCAASGFTLNSYGMHWVRQTPGKGLEWVANIWKDGINKYYADSVMGRVTISRDNSRNTLYLQMNSLRAEDTAVYFCARDLDYSGMDVWGQGT

SEQ ID NO: 49 - DNA, 333 bp, Homo sapiens; identical to SEQ ID NO: 23

SEQ ID NO: 50 - protein, 111 aa, Homo sapiens; identical to SEQ ID NO: 24

SEQ ID NO: 51 - DNA, 333 bp, Homo sapiens; identical to SEQ ID NO: 23

SEQ ID NO: 52 - protein, 111 aa, Homo sapiens; identical to SEQ ID NO: 24

SEQ ID NO: 53 - DNA, 303 bp, Homo sapiens
gagctcacgcagtctccagccaccgtgtctgtgtctccaggggaaagagccaccctctcctgcagggccagtcagagtattagtttccacttagcctggtaccagcagaaacctggccaggctcccagtctcctcatctacggaacatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 54 - protein, 101 aa, Homo sapiens
ELTQSPATVSVSPGERATLSCRASQSISFHLAWYQQKPGQAPSLLIYGTSTRATGIPARFSGSGSGTEFTLTISSLQSEDSAVYYCQQYHNWPPLTFGGGT

SEQ ID NO: 55 - DNA, 303 bp, Homo sapiens
gagctcacgcagtctccagccaccgtgtctgtgtctccaggggaaagagccaccctctcctgcagggccagtcagagtattagtttccacttagcctggtaccagcagaaacctggccaggctcccaggctcctcatctatggggcatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 56 - protein, 101 aa, Homo sapiens
ELTQSPATVSVSPGERATLSCRASQSISFHLAWYQQKPGQAPRLLIYGASTRATGIPARFSGSGSGTEFTLTISSLQSEDSAVYYCQQYHNWPPLTFGGGT

SEQ ID NO: 57 - DNA, 285 bp, Homo sapiens
gccaccctgtctctgtctccaggggatagagccaccctctcctgcagggccagtcagagtattagtttccacttagcctggtaccagcagaaacctggccaggctcccaggctcctcatctatggggcatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 58 - protein, 95 aa, Homo sapiens
ATLSLSPGDRATLSCRASQSISFHLAWYQQKPGQAPRLLIYGASTRATGIPARFSGSGSGTEFTLTISSLQSEDSAVYYCQQYHNWPPLTFGGGT

SEQ ID NO: 59 - DNA, 285 bp, Homo sapiens
gccaccgtgtctgtgtctccaggggaaagagccaccctctcctgcagggccagtcagagtattagtttccacttagcctggtaccagcagaaacctggccaggctcccaggctcctcatctatggggcatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 60 - protein, 95 aa, Homo sapiens
ATVSVSPGERATLSCRASQSISFHLAWYQQKPGQAPRLLIYGASTRATGIPARFSGSGSGTEFTLTISSLQSEDSAVYYCQQYHNWPPLTFGGGT

SEQ ID NO: 61 - DNA, 288 bp, Homo sapiens
ccagccaccgtgtctgtgtctccaggggaaagagccaccctctcctgcagggccagtcagagtgttagcagtaacttagcctggtaccagcagaaacgtggccaggctcccagtctcctcatctacggaacatctaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 62 - protein, 96 aa, Homo sapiens
PATVSVSPGERATLSCRASQSVSSNLAWYQQKRGQAPSLLIYGTSTRATGIPARFSGSGSGTEFTLTISSLQSEDSAVYYCQQYHNWPPLTFGGGT

SEQ ID NO: 63 - DNA, 288 bp, Homo sapiens
ccagccaccgtgtctgtgtctccaggggaaagagccaccctctcctgcagggccagtcagagtattagtttccacttagcctggtaccagcagaaacctggccaggctcccagtctcctcatctacggaacatctaccagggccactggtatcccagccaggttcagtggcagtgggtctgggacagagttcactctcaccatcagcagcctgcagtctgaagattttgcagtttattactgtcagcagtatcataactggcctcccctcactttcggcggagggacc

SEQ ID NO: 64 - protein, 96 aa, Homo sapiens
PATVSVSPGERATLSCRASQSISFHLAWYQQKPGQAPSLLIYGTSTRATGIPARFSGSGSGTEFTLTISSLQSEDFAVYYCQQYHNWPPLTFGGGT

SEQ ID NO: 65 - DNA, 454 bp, Homo sapiens
ctcgaggagtctggcccaggactggtgaagccttcggagaccctgtccctcacctgcactgtctctggtgactccatgagtagttattattggaactggatccggcagtccccagggaagggactggaatggattggatatatctattacaatgggaactccaactacaacccctccctcaggagtcgagtcaccatatcaattgacacgtccaagaagcagttctccctgaagctgacctctgcgaccgccgcagacacggccgtttatttctgtgcggggacggaatatgattatctttgggggacccccaatacggatgcatttgatatctggggccgagggacagtggtcgccgtctcctcagcctccaccaagggcccatcggtcttccccctggcaccctcctccaagagcacctctgggggcacagcggccctgggctgcctggtca

SEQ ID NO: 66 - protein, 151 aa, Homo sapiens
LEESGPGLVKPSETLSLTCTVSGDSMSSYYWNWIRQSPGKGLEWIGYIYYNGNSNYNPSLRSRVTISIDTSKKQFSLKLTSATAADTAVYFCAGTEYDYLWGTPNTDAFDIWGRGTVVAVSSASTKGPSVFPLAPSSKSTSGGTAALGCLV

SEQ ID NO: 67 - DNA, 445 bp, Homo sapiens
ctcgaggagtctgggggaggcgtggtccagcctgggaggtccctaagactctcctgtgcagcctctggattcaccttcagtagctatggcatgcactgggtccgccaggctccaggcaaggggctggagtgggtggcagtgatatcgtatgatggaagtaataaaaagtatgcagactctgtgaagggccgattcaccatctccagagacgattccaagaaaacgctgtatctgcaaatgaacagtatgagacgtgaggacacggctgtgtatttctgtgcgaaagcggcgaatacagtaggtcgtccaggatggttcgacccctggggccagggaaccctggtcaccgtctcctcagcctccaccaagggcccatcggtcttccccctggcaccctcctccaagagcacctctgggggcacagcggccctgggctgcctggtca

SEQ ID NO: 68 - protein, 148 aa, Homo sapiens
LEESGGGVVQPGRSLRLSCAASGFTFSSYGMHWVRQAPGKGLEWVAVISYDGSNKKYADSVKGRFTISRDDSKKTLYLQMNSMRREDTAVYFCAKAANTVGRPGWFDPWGQGTLVTVSSASTKGPSVFPLAPSSKSTSGGTAALGCLV

SEQ ID NO: 69 - DNA, 433 bp, Homo sapiens
ctcgagtcggggggaggcgcgatacagccgggggagtccctgagactcttctgtgcagcctctggattcacctttcgcgactatgccatgggctgggtccgccgggctccagggaagggactggagtgggtctcatctatcaatgatagtggtgatagaacatattacgcagactccgtgaagggccgcttcaccatctccagagacaactccaagaattctctttatctgcaaatgaccagcctgagagccgcggacacggccatatattactgtgcgaaaggcttgatcggtctctcatcttttcatgtctggggccaagggacactggtcaccgtctcttcagcctccaccaagggcccatcggtcttccccctggcaccctcctccaagagcacctctgggggcacagcggccctgggctgcctggtca

SEQ ID NO: 70 - protein, 144 aa, Homo sapiens
LESGGGAIQPGESLRLFCAASGFTFRDYAMGWVRRAPGKGLEWVSSINDSGDRTYYADSVKGRFTISRDNSKNSLYLQMTSLRAADTAIYYCAKGLIGLSSFHVWGQGTLVTVSSASTKGPSVFPLAPSSKSTSGGTAALGCLV

SEQ ID NO: 71 - DNA, 457 bp, Homo sapiens
ctcgagtctgggggaggcttggtacagccgggggggtccctgagactatcttgtgcagcctctggattcacctttagaaggcattccatgagttgggtccgccaggctccagggaaggggctggagtggatctcagctattagtggtagtgctggtagttcatactacgcagactccgtgaagggccggttcaccatttccagagacaatttcaagaacacattatatctgcaaatgaacagcctgcgacccgaggacacggccatatattattgtgcgaaaagagtgtctgcttaccttattggggattactcctttaactactacatagacgtctggggcacagggaccacggtcaccgtctcctcagcttccaccaagggcccatcggtcttccccctggcgccctgctccaggagcacctctgggggcacagcggccctgggctgcctggtca

SEQ ID NO: 72 - protein, 152 aa, Homo sapiens
LESGGGLVQPGGSLRLSCAASGFTFRRHSMSWVRQAPGKGLEWISAISGSAGSSYYADSVKGRFTISRDNFKNTLYLQMNSLRPEDTAIYYCAKRVSAYLIGDYSFNYYIDVWGTGTTVTVSSASTKGPSVFPLAPCSRSTSGGTAALGCLV

SEQ ID NO: 73 - DNA, 433 bp, Homo sapiens
ctcgagtcgggcccaggactggtgaagccttcacagaccctgtccctcacttgcacagtctctggcggctccgtcagaagtggtggttactactggagctggatccgtcacctcccagggaagggcctggagtggattgggtgcaccttttacgggggaaggacctactacagcccgtccctcaagagtcgagttaccatatcgacagacacgtctaagaaccagttctccctgaggctgacctctgtgactgccgcggacacggccgtgtattattgtgcgagagatgatggcggtagacccatagacgtctggggcagagggaccacggtcgccgtctcctcagcctccaccaagggcccatcggtcttccccctggcaccctcctccaagagcacctctgggggcacagcggccctgggctgcctggtca

SEQ ID NO: 74 - protein, 144 aa, Homo sapiens
LESGPGLVKPSQTLSLTCTVSGGSVRSGGYYWSWIRHLPGKGLEWIGCTFYGGRTYYSPSLKSRVTISTDTSKNQFSLRLTSVTAADTAVYYCARDDGGRPIDVWGRGTTVAVSSASTKGPSVFPLAPSSKSTSGGTAALGCLV

SEQ ID NO: 75 - DNA, 451 bp, Homo sapiens
ctcgagcagtctgggggaggcgtggtccagcccggggggtccctgagactctcctgtgcagcctctggattcaccttcagtagccatggcatgaactgggtccgccaggctccagggaaggggctggagtggcttgcattcataagtggtagtggtgataccatatttgacgccgactccgtgaagggccgattcaccatctccagagacaacgccgggaacttattgtatctggaaatgaacagcctgcgagccgaggacacggctgtatattactgtgcaagagatcataccaggtgctattccttgagggggtgcggtatggacgtctggggccaagggaccacggtcaccgtcgcctcagcctccaccaagggcccatcggtcttccccctggcaccctcctccaagagcacctctgggggcacagcggccctgggctgcctggtca

SEQ ID NO: 76 - protein, 150 aa, Homo sapiens
LEQSGGGVVQPGGSLRLSCAASGFTFSSHGMNWVRQAPGKGLEWLAFISGSGDTIFDADSVKGRFTISRDNAGNLLYLEMNSLRAEDTAVYYCARDHTRCYSLRGCGMDVWGQGTTVTVASASTKGPSVFPLAPSSKSTSGGTAALGCLV

SEQ ID NO: 77 - DNA, 449 bp, Homo sapiens
ctcgaggagtctgggggaggcgtggtccagcctgggaggtccctgagactctcctgtgcagcctctggattcactttcaagaattatgccatgcactgggtccgccaggctccaggcaaggggctggagtgggtggcagttatatcatatgatgggaccaatgaatactacgcagactccgtgaagggccgattcaccatctccagagacaattccaagaacacactgtatctgcaaatgagcagcctgagacttgaggacacgtctgtgttttactgtgcgagagacgtcccgcctaaatcgccctgggtgccagctgccctctattggggccggggaaccctggtcaccgtctcctcagcctccaccaagggcccatcggtcttccccctggcacccctcctccaagagcacctctgggggcacagcggccctgggctgcctggtca

SEQ ID NO: 78 - protein, 149 aa, Homo sapiens
LEESGGGVVQPGRSLRLSCAASGFTFKNYAMHWVRQAPGKGLEWVAVISYDGTNEYYADSVKGRFTISRDNSKNTLYLQMSSLRLEDTSVFYCARDVPPKSPWVPAALYWGRGTLVTVSSASTKGPSVFPLAPLLQEHLWGHSGPGLPG

SEQ ID NO: 79 - DNA, 439 bp, Homo sapiens
ctcgaggagtctggggctgaggtgaagaagcctgggtcctcggtgaagatctcctgcaaggcttctggagacaccttcaacacttttactatcacctgggtgcgacaggcccctggacaaggacttgagtggatggggaggatcagccctatccctgatataacaaattacgcacagaacttccaggmcagagtcaaaatcaccgcggacaaatccacgagaacagcctacatggaattgagcagtctgagatctgacgacacggccgtctattattgtgcgagagagcgatcgatggcccggaatggcttggycgtctggggccaagggaccacggtcatcgtctcctcagcctccaccaagggcccatcggtcttccccctggcaccctcctccaagagcacctctgggggcacagcggccctgggctgcctggtca

SEQ ID NO: 80 - protein, 143 aa, Homo sapiens
LEESGAEVKKPGSSVKISCKASGDTFNTFTITWVRQAPGQGLEWMGRISPIPDITNYAQNFQARVKITADKSTRTAYMELSSLRSDDTAVYYCARERSMARAVWGQGTTVIVSSASTKGPSVFPLAPSSKSTSGGTAALGCLV

SEQ ID NO: 81 - DNA, 442 bp, Homo sapiens
ctcsagcagtctgggggaggcgtggtccagcctgagaggtccctgagactctcctgtgcagcctctggattcagtttcagtagttcttctatgcactgggtccgccaggctccaggcaaggggctggagtgggtggccgttatatcatatgatggaagcaatgaacactatgcagactccgtgaagggccgtttcaccatctccagagacaattccaagaacacggtgtatctgcaaatgaacagcctgacacctgcggacacggctgcgtatttctgtgcgagagggggatggctccaaatacaatactactttgactactggggccaaggaaccctggtcaccgtctcctcagcctccaccaagggcccatcggtcttccccctggcaccctcctccaagagcacctctgggggcacagcggccctgggctgcctggtca

SEQ ID NO: 82 - protein, 147 aa, Homo sapiens
LEQSGGGVVQPERSLRLSCAASGFSFSSSSMHWVRQAPGKGLEWVAVISYDGSNEHYADSVKGRFTISRDNSKNTVYLQMNSLTPADTAAYFCARGGWLQIQYYFDYWGQGTLVTVSSASTKGPSVFPLAPSSKSTSGGTAALGCLV

SEQ ID NO: 83 - DNA, 445 bp, Homo sapiens
ctcgagtcgggcccaggactgctgaagccttcggagaccctgtcactcacctgcactgtctctggtggctccatcagttcctactactggacctggatccggcagaccccagggaagggactggagtggattgggtctatctctgacagtgggagcgccagctacaacccctccctcaagagtcgagtcactatatcagtggacacgtccacgaaccagttctccctgaagctgacctctgtgtccgccgcagacacggccgtatactactgtgcgagacatgtaaatatagattacgcttataacctaaattactttcactactggggccagggaaccctggtcaccgtctcctcagcctccaccaagggcccatcggtcttccccctggcaccctcctccaagagsacctctgggggcacagcggccctgggctgcctggtca

SEQ ID NO: 84 - protein, 148 aa, Homo sapiens
LESGPGLLKPSETLSLTCTVSGGSISSYYWTWIRQTPGKGLEWIGSISDSGSASYNPSLKSRVTISVDTSTNQFSLKLTSVSAADTAVYYCARHVNIDYAYNLNYFHYWGQGTLVTVSSASTKGPSVFPLAPSSKSTSGGTAALGCLV

SEQ ID NO: 85 - DNA, 454 bp, Homo sapiens
ctcgagtcgggcccaggactggtgaagccttcggagaccctgtccctcacttgcaatgtctctggaggctccatgaagaattacttctgggcctggatccggcagcccgcagggaagggactggagtggattgggtatatctattacagtgggaccaccaactacaacccctccctcaagagtcgagtcaccatatcagtggacacgtccgagaaccaattctccctgaggctgagctctgtgtccgccgcagacacggccgtctattattgtgcgagacttgtcggccccgattattggagtggtgtcaactacttctacggaatggacgtctggggccaagggaccacggtcaccgtctcctccgcctccaccaagggcccatcggtcttccccctggcaccctcctccaagagcacctctgggggcacagcggccctgggctgcctggtca

SEQ ID NO: 86 - protein, 151 aa, Homo sapiens
LESGPGLVKPSETLSLTCNVSGGSMKNYFWAWIRQPAGKGLEWIGYIYYSGTTNYNPSLKSRVTISVDTSENQFSLRLSSVSAADTAVYYCARLVGPDYWSGVNYFYGMDVWGQGTTVTVSSASTKGPSVFPLAPSSKSTSGGTAALGCLV

SEQ ID NO: 87 - DNA, 415 bp, Homo sapiens
ctcgagtctgggggaggcgtggtccagcctgggaggtccctgaaactctcctgtgcagcgtctggattcaccttcactactcatggcatgcactgggtccgccagtctccaggcaaggggctggagtgggtggcagttatacggtctgatggaaagactaaatactatgcagactccgtgaagggccgattcaccatatccagagacgattcgaagaacacgctatatctgcaaatgaacagcctgagagccgaggacacggctgtctactactgtgcgagaaatctccaagactggggccagggaaccctggtcaccgtctcctcagcctccaccaagggcccatcggtcttccccctggcaccctcctccaagagcacctctgggggcacagcggccctgggctgcctggtca

SEQ ID NO: 88 - protein, 138 aa, Homo sapiens
LESGGGVVQPGRSLKLSCAASGFTFTTHGMHWVRQSPGKGLEWVAVIRSDGKTKYYADSVKGRFTISRDDSKNTLYLQMNSLRAEDTAVYYCARNLQDWGQGTLVTVSSASTKGPSVFPLAPSSKSTSGGTAALGCLV

SEQ ID NO: 89 - DNA, 436 bp, Homo sapiens
ctcgagtggggcgcaggactgttgaagccttcggagaccctgtccctcacctgcgcagtcgatgagaggaccttcagtgatgactactggagctggatccgccagcccccagggaaggggctggagtggattggggagatcaataaaagtggaatatccacctacaacccgtccctgacgagtcgagtcaccatattattagacatgtccaagaggcagttctccctgaggctgagctctgtgaccgccgcggacacggctgtgtattattgtgcaagaaacgtggatcagggagatagtgcccactttgactactggggccagggaacccaggtcaccgtctcctcagcctccaccaagggcccatcggtcttccccctggcaccctcctccaagagcacctctgggggcacagcggccctgggctgcctggtca

SEQ ID NO: 90 - protein, 145 aa, Homo sapiens
LEWGAGLLKPSETLSLTCAVDERTFSDDYWSWIRQPPGKGLEWIGEINKSGISTYNPSLTSRVTILLDMSKRQFSLRLSSVTAADTAVYYCARNVDQGDSAHFDYWGQGTQVTVSSASTKGPSVFPLAPSSKSTSGGTAALGCLV

SEQ ID NO: 91 - DNA, 457 bp, Homo sapiens
gaggagtctgggggaggcttgggacagccgggggggtccctgagactctcctgtgaagtgtctggattcacctttagcagctatgccgtgacctgggtccgccaggctccagggaaggggctacagtgggtctcaactatcagtggttctggtgaaaacacatactacgcagactccgtgaggggccggtttaccgtctccagagacaattccaagaacactctgtatctacaaatgaacagcctgagagccgaggacacggccgtttatttctgtgcgagagtgccctataacgatatcttgcaccgctttctacaccagccttactttgactgctggggccagggaaccctggtcaccgtctcctcagcttccaccaagggcccatcggtcttccccctggcgccctgctccaggagcacctctgggggcacagcggccctgggctgcctggtca

SEQ ID NO: 92 - protein, 152 aa, Homo sapiens
EESGGGLGQPGGSLRLSCEVSGFTFSSYAVTWVRQAPGKGLQWVSTISGSGENTYYADSVRGRFTVSRDNSKNTLYLQMNSLRAEDTAVYFCARVPYNDILHRFLHQPYFDCWGQGTLVTVSSASTKGPSVFPLAPCSRSTSGGTAALGCLV

SEQ ID NO: 93 - DNA, 442 bp, Homo sapiens
ctcgagcagtctgggggaggcttggtcaagcctggaggatccctgagactctcctgtgcgggctctggattcacgttcaatgactactacctggcttggatccgccaggctccagggaaggggctggagtggcttgcattcattagtagcagtggttcttccatatactatgccgactctctgaagggccgattcaccatctccagggacaacgtccggaagtctctgtctctgcaaatgaacagcctgagagtcgaggacacggccgtatatttctgtgcgagagtcgttgtaccgacggacgaatattacatggacgtctggggcaaagggaccacggtcaccgtctcctcagcctccaccaagggcccatcggtcttccccctggcaccctcctccaagagcacctctgggggcacagcggccctgggctgcctggtca

SEQ ID NO: 94 - protein, 147 aa, Homo sapiens
LEQSGGGLVKPGGSLRLSCAGSGFTFNDYYLAWIRQAPGKGLEWLAFISSSGSSIYYADSLKGRFTISRDNVRKSLSLQMNSLRVEDTAVYFCARVVVPTDEYYMDVWGKGTTVTVSSASTKGPSVFPLAPSSKSTSGGTAALGCLV

SEQ ID NO: 95 - DNA, 460 bp, Homo sapiens
ctcgagtggggcgcaggactgttgaagccttcggagaccctgtccctcacctgcgctgtctatggtgggtccttcagtgattactactggagctggatccgccagcccccagggaaggggctggagtggattggggaaatcaatcatagtggacgcaccaagtacaacccgtccctcaagagtsgagtcaccatatcagtagacacgtccaagaaccagttctccctgaagctgagctctgtgaccgccgcggacacggctgtatattactgtgcgagagtctyttccccccgtattacgatttttgaagtggtattccgctactactacatggacgtctggggcaaagggaccacggtcaccgtctcctcagcttccaccaagggcccatcggtcttccccctggcgccctgctccaggagcacctctgggggcacagcggccctgggctgcctggtca

SEQ ID NO: 96 - protein, 153 aa, Homo sapiens
LEWGAGLLKPSETLSLTCAVYGGSFSDYYWSWIRQPPGKGLEWIGEINHSGRTKYNPSLKSRVTISVDTSKNQFSLKLSSVTAADTAVYYCARVSSPRITIFEVVFRYYYMDVWGKGTTVTVSSASTKGPSVFPLAPCSRSTSGGTAALGCLV

SEQ ID NO: 97 - DNA, 430 bp, Homo sapiens
ctcgagtggggcgcggggctcttgaagccttcggagaccctgtccctcacctgcgctgtctatggtgggtccttcagtggttactactggacctggatccgccagtccccagggaaggggctggagtggattggggaaatcaatcaaagtggaagcacccactacaacccgtcgttgaacagtcgagtcaccatatcagtagacacgtctaagaaccagatcttcctgaacgtgaactctgtgaccgccgcggacacggctatgtattactgtgcgagatactcgaatatgggtggctggttggacccctggggccagggaaccctggtcatcgtctcctcagcctccaccaagggcccatcggtcttccccctggcaccctcctccaagagcacctctgggggcacagcggccctgggctgcctggtca

SEQ ID NO: 98 - protein, 143 aa, Homo sapiens
LEWGAGLLKPSETLSLTCAVYGGSFSGYYWTWIRQSPGKGLEWIGEINQSGSTHYNPSLNSRVTISVDTSKNQIFLNVNSVTAADTAMYYCARYSNMGGWLDPWGQGTLVIVSSASTKGPSVFPLAPSSKSTSGGTAALGCLV

SEQ ID NO: 99 - DNA, 466 bp, Homo sapiens
ctcgagtctgggggaggcctggtcaagcctggggggtccctgagactctcctgtgcagcctctggcttcagcttcagtgattatactatgaactgggtccgccaggctccagggagggggctggagtgggtctcatcaataagaagcactagtccttacatattctacgcagactcagtgaagggccgattcaccatctccagagacaacgccgcaaactcactgtatctgcaaatgaacagcctgcgagccgaggacacggctgtctattactgtgcgagcgcccgccctgttagtatgattcggggagttcccccccgctacaattaccacggtatggacgtctggggcctggggaccacggtcaccgtctcctcagcctccaccaagggcccatcggtcttccccctggcaccctcctccaagagcacctctgggggcacagcggccctgggctgcctggtca

SEQ ID NO: 100 - protein, 155 aa, Homo sapiens
LESGGGLVKPGGSLRLSCAASGFSFSDYTMNWVRQAPGRGLEWVSSIRSTSPYIFYADSVKGRFTISRDNAANSLYLQMNSLRAEDTAVYYCASARPVSMIRGVPPRYNYHGMDVWGLGTTVTVSSASTKGPSVFPLAPSSKSTSGGTAALGCLV

SEQ ID NO: 101 - DNA, 466 bp, Homo sapiens
ctcgagtctgggggaggcctggtcaagcctggggggtccctgagactctcctgtgcagcctctggcttcagcttcagtgattatactatgaactgggtccgccaggctccagggagggggctggagtgggtctcatcaataagaagcactagtccttacatattctacgcagactcagtgaagggccgattcaccatctccagagacaatgccgcaaactcactgtatctgcaaatgaacagcctgcgagccgaggacacggctgtctattactgtgcgagcgcccgccctgttagtatgattcggggagttcccccccgctacaattaccacggtatggacgtctggggcctggggaccacggtcaccgtctcctcagcctccaccaagggcccatcggtcttccccctggcaccctcctccaagagcacctctgggggcacagcggccctgggctgcctggtca

SEQ ID NO: 102 - protein, 155 aa, Homo sapiens; identical to SEQ ID NO: 100

SEQ ID NO: 103 - DNA, 445 bp, Homo sapiens
ctcgagcagtctgggggaggcttggtaaaccctggggggtcccttagactctcctgtgcagcctctggattcactttcagtaaggcctggatgacctgggtccgccaggctccagggaaggggctggagtgggttggccgtattaaaagcatgactgatagtgggacaacagactacgctgcacccgtgaaaggccgattctccatctccagagacgattcaaaaaacatgctgtatttgcaaatgagcagcctgaaaaccgaggacacagccgtgtattactgtgccacagatccaagggcacacccggatgcttttgatatctggggccaagggacaatggtcaccgtctcttcagcctccaccaagggcccatcggtcttccccctggcaccctcctccaagagcacctctgggggcacagcggccctgggctgcctggtca

SEQ ID NO: 104 - protein, 148 aa, Homo sapiens
LEQSGGGLVNPGGSLRLSCAASGFTFSKAWMTWVRQAPGKGLEWVGRIKSMTDSGTTDYAAPVKGRFSISRDDSKNMLYLQMSSLKTEDTAVYYCATDPRAHPDAFDIWGQGTMVTVSSASTKGPSVFPLAPSSKSTSGGTAALGCLV

SEQ ID NO: 105 - DNA, 436 bp, Homo sapiens
ctcgagtcggggggaggcttggtccagcctggggggtccctgagactctcctgtgcagcctctggattcaccttcagtaattatgctatacattgggtccgccaggctccagggaagggactggaatatgtttcagctattagtagcaatggggatagcacatattatgcaaagtctgtgaacggcagattcaccatctccagagaccattccaagaacacgctgtatcttcagatgggcagcctgagagctgaggacatggctgtgtattactgtgtgaggtcccccctcctacgatattctaaaatggacgtctggggccaagggaccacggtcaccgtctcctcagcctccaccaagggcccatcggtcttccccctggcaccctcctccaagagcacctctgggggcacagcggccctgggctgcctggtca

SEQ ID NO: 106 - protein, 145 aa, Homo sapiens
LESGGGLVQPGGSLRLSCAASGFTFSNYAIHWVRQAPGKGLEYVSAISSNGDSTYYAKSVNGRFTISRDHSKNTLYLQMGSLRAEDMAVYYCVRSPLLRYSKMDVWGQGTTVTVSSASTKGPSVFPLAPSSKSTSGGTAALGCLV

SEQ ID NO: 107 - DNA, 320 bp, Homo sapiens
tctccttccaccctgtctgcatctgtcggcgacagagtcaccatcacttgccgggccagtcaaagtattagaacctggttggcctggtatcagcagaaaccagggaaagcccctaacctcctgatctatcatgcctccagtttggaaagtggggtcccatcaaggttcagcggtaatggatctgggacggaattcactctcaccatcaacagcctgcagcctgatgattttgcaacttattactgccaacactatattacttattcgtggacgttcggccaagggaccaaggtggaaatcaaacgaactgtggctgcacc

SEQ ID NO: 108 - protein, 106 aa, Homo sapiens
SPSTLSASVGDRVTITCRASQSIRTWLAWYQQKPGKAPNLLIYHASSLESGVPSRFSGNGSGTEFTLTINSLQPDDFATYYCQHYITYSWTFGQGTKVEIKRTVAA

SEQ ID NO: 109 - DNA, 320 bp, Homo sapiens
tctccttccaccctgtctgcatctgtaggagacagagtcaccgtcacttgccgggccagtcagagtattagtagctggttggcctggtatcagcagaaaccagggaaagcccctaaactcctgatctatgatgcctccagtttgcaaagtggggtcccatcaaggttcagcggcggtggatctgggacagaattcactctcaccatcaacagcctgcagcctgatgattttgcaacttattactgccaacagtataatagttatccgtggacgttcggccaagggaccaaggtggaaatcaaacgaactgtggctgcacc

SEQ ID NO: 110 - protein, 106 aa, Homo sapiens
SPSTLSASVGDRVTVTCRASQSISSWLAWYQQKPGKAPKLLIYDASSLQSGVPSRFSGGGSGTEFTLTINSLQPDDFATYYCQQYNSYPWTFGQGTKVEIKRTVAA

SEQ ID NO: 111 - DNA, 320 bp, Homo sapiens
tctccttccaccctgtctgcatctgtaggagacaaagtcaccatcacttgccgggccagtcagagtattagtaggtgggtggcctggtatcagcagaaaccagggacagcccctaaggtcctgatctatgatgcctccaggttggaaagtggggtcccatcaaggttcagcggcagtggatctgggacagaattcactctcaccatcagtagcctgcagcctgatgattttgcaacttattactgccaacagtataatagttatttgtggacgttcggccaagggaccaaggtggaaatcaaacgaactgtggctgcacc

SEQ ID NO: 112 - protein, 106 aa, Homo sapiens
SPSTLSASVGDKVTITCRASQSISRWVAWYQQKPGTAPKVLIYDASRLESGVPSRFSGSGSGTEFTLTISSLQPDDFATYYCQQYNSYLWTFGQGTKVEIKRTVAA

SEQ ID NO: 113 - DNA, 320 bp, Homo sapiens
tctccaccctccctgtctgcatttgttggagacaaagtcaccatcacttgccgggcaagtcaggacatttacagagctttagcctggtatcagcagaagccagggaagcctcctaacctcttgatctatcatgcctccagtttgcaaagaggggtcccatcaaggttcagcggcagtggatctgggacagatttcactctcaccatcagcagcctgcagcctgaagattttgcaacttattactgtcaacagtttaatagttttccgctcactttcggcggacggaccaacgtggagatcaaccgaactgtggctgcacc

SEQ ID NO: 114 - protein, 106 aa, Homo sapiens
SPPSLSAFVGDKVTITCRASQDIYRALAWYQQKPGKPPNLLIYHASSLQRGVPSRFSGSGSGTDFTLTISSLQPEDFATYYCQQFNSFPLTFGGRTNVEINRTVAA

SEQ ID NO: 115 - DNA, 320 bp, Homo sapiens
tctccatcctccctgtctgcatctgtaggagacagaatcaccatcacttgccgggccagtcaagacattagtagttatttagggtggtatcagcagaaaccagggacggcccctaagctcctgatatatgctgcatccactttgcacagtggggtcccatcaaggttcagcggcagtggatctgggacagaattcactctcacaatcaataggctgcagcctgaagactttgcagcttattactgtcaacaggttgacagttatcctcgaactttcggcggagggaccaaggtggagatgaaacgaactgtggctgcacc

SEQ ID NO: 116 - protein, 106 aa, Homo sapiens
SPSSLSASVGDRITITCRASQDISSYLGWYQQKPGTAPKLLIYAASTLHSGVPSRFSGSGSGTEFTLTINRLQPEDFAAYYCQQVDSYPRTFGGGTKVEMKRTVAA

SEQ ID NO: 117 - DNA, 320 bp, Homo sapiens
tctccatccatcctgtctgcatctgtaggagacagagtcaccatcacttgccgggccagtcaagacattagtagttatttagggtggtatcagcagaaaccagggacggcccctaagctcctgatatatgctgcatccactttgcacagtggggtcccatcaaggttcagcggcagtggatctgggacagaattcactctcacaatcaataggctgcagcctgaagactttgcagcttattactgtcaacaggttgacagttatcctcgaactttcggcggagggaccaaggtggagatgaaacgaactgtggctgcacc

SEQ ID NO: 118 - protein, 106 aa, Homo sapiens
SPSILSASVGDRVTITCRASQDISSYLGWYQQKPGTAPKLLIYAASTLHSGVPSRFSGSGSGTEFTLTINRLQPEDFAAYYCQQVDSYPRTFGGGTKVEMKRTVAA

SEQ ID NO: 119 - DNA, 320 bp, Homo sapiens
tctccagcctccctatatgcgtctgttggcgacaccgtcaccatcacttgccgggcaagtcagggcattagaaaagctttaggctggtatcagcagaaacaagggggagcccctaagtgcctgctctatgctgcattcactttgcaaactggagtcccaccaaggttcagcggcagtggatctgggacagaattcactctcacaatcagtagcctgcagcctgaagactttgcaacttattactgtctacagcataattcttatccttggacgttcggccaagggaccaaggtggaaatcaaacgaactgtggctgcacc

SEQ ID NO: 120 - protein, 106 aa, Homo sapiens
SPASLYASVGDTVTITCRASQGIRKALGWYQQKQGGAPKCLLYAAFTLQTGVPPRFSGSGSGTEFTLTISSLQPEDFATYYCLQHNSYPWTFGQGTKVEIKRTVAA

SEQ ID NO: 121 - DNA, 317 bp, Homo sapiens
tctccatcctccctgtctgcatctgtaggagacagaatcaccatcacttgccgggcaagtcagagaattagtacctatgtaaattggtatcagcagaagccagggaaaggccctaagctcctgatctatgccgcatccagtttgcaaagtggggtcccatcaaggttcagtggcagtggatctgggacagatttcactctcaccatcagcagtctccagtctgaagactttgcaacttactactgtcaacagagttacatatcttggacgttcggccaagggaccaaggtggaaatcaaacgaactgtggctgcacc

SEQ ID NO: 122 - protein, 105 aa, Homo sapiens
SPSSLSASVGDRITITCRASQRISTYVNWYQQKPGKGPKLLIYAASSLQSGVPSRFSGSGSGTDFTLTISSLQSEDFATYYCQQSYISWTFGQGTKVEIKRTVAA

SEQ ID NO: 123 - DNA, 320 bp, Homo sapiens
tctccatcctccctgtctgcatctgtaggagacagagtcaccatcacttgccgggcaagtcagagcgttacgagatatttaaattggtatcagcagaaaccaggcaaagcccctaaggtcctgatctatgctgcatccagtttgcaaagtggggtcccatcaaggttcagtggcagtggatctgggacagatttcactctcaccatcagcagtctgcaacctgaagattttgcatcttactactgtcaacagagttacagtaccccttggacgttcggccaagggaccaaggtggaaatcaaacgaactgtggctgcacc

SEQ ID NO: 124 - protein, 106 aa, Homo sapiens
SPSSLSASVGDRVTITCRASQSVTRYLNWYQQKPGKAPKVLIYAASSLQSGVPSRFSGSGSGTDFTLTISSLQPEDFASYYCQQSYSTPWTFGQGTKVEIKRTVAA

SEQ ID NO: 125 - DNA, 320 bp, Homo sapiens
tctccatcctccctgtctgcatctgtaggagacagagtcaccatcacttgccgggcaagtcagagcattagcagctatttaaattggtatcaacagaaaccagggaaagcccctaagctcctgataaatgttgcatccagtttgcaaagtggggtcccatcaaggttcagtggcagtgcatctgggacaaatttcactctcaccatcagcagtctgcaacctgaagattttgcaacttactactgtcagcagacttacaggagccctaggacgttcggccaagggaccaaggtggaaatcaaacgaactgtggctgcacc

SEQ ID NO: 126 - protein, 106 aa, Homo sapiens
SPSSLSASVGDRVTITCRASQSISSYLNWYQQKPGKAPKLLINVASSLQSGVPSRFSGSASGTNFTLTISSLQPEDFATYYCQQTYRSPRTFGQGTKVEIKRTVAA

SEQ ID NO: 127 - DNA, 320 bp, Homo sapiens
tctccatcctccctgtctgcatctgtaggagacagagtcaccatcagttgccgggcaagtcagagcattagcgactatttacattggtatcagcagaaaccagggaaagcccctaacctcctgatctatgctgcatccaatttgcacagtggggtcccatcgaggttcagtggcagtggatctgggacagatttcactctcaccatcaacagtctgcaacctgaagattttgcaacttactactgtcaacagagtttctctaccccgtggacgttcggccacgggaccaaggtggaaatcaaacgaactgtggctgcacc

SEQ ID NO: 128 - protein, 106 aa, Homo sapiens
SPSSLSASVGDRVTISCRASQSISDYLHWYQQKPGKAPNLLIYAASNLHSGVPSRFSGSGSGTDFTLTINSLQPEDFATYYCQQSFSTPWTFGHGTKVEIKRTVAA

SEQ ID NO: 129 - DNA, 320 bp, Homo sapiens
tctccatcctccctgtctgcatctgttggagacagagtcaccatcacttgccgggcaagtcagagcatcagcaactatttaaattggtatcagcagaaaccagggaaagcccctaaactcctgatctctggtgcatccagtttgcagagtggggtcccatctaggttcagtggcagtggatttgggacagatttcagtctcaccatcaactttctgcaatctgaagattttgctgtttactactgtcaacagggttacagcaccccgtacacttttggccaggggaccaagctggagatgaaacgaactgtggctgcacc

SEQ ID NO: 130 - protein, 106 aa, Homo sapiens
SPSSLSASVGDRVTITCRASQSISNYLNWYQQKPGKAPKLLISGASSLQSGVPSRFSGSGFGTDFSLTINFLQSEDFAVYYCQQGYSTPYTFGQGTKLEMKRTVAA

SEQ ID NO: 131 - DNA, 320 bp, Homo sapiens
tctccatcctccctggctgcatctgtcggaaacagagtcgccatcacttgccgggcaagtcagagcatttccaactatgtaaattggtatcagcagaaaccagggaaagcccctaacctcctaatctctgctgcatccaatttacaaagtggggtcccatcaaggttcactggcagtggatctgggacagatttcactctcaccatcagtagtctggaacctgaagattttgcaacttactactgtcaacagagtcaggctgcccctctcactttcggcggagggaccaaggtggagatcaagcgaactgtggctgcacc

SEQ ID NO: 132 - protein, 106 aa, Homo sapiens
SPSSLAASVGNRVAITCRASQSISNYVNWYQQKPGKAPNLLISAASNLQSGVPSRFTGSGSGTDFTLTISSLEPEDFATYYCQQSQAAPLTFGGGTKVEIKRTVAA

SEQ ID NO: 133 - DNA, 320 bp, Homo sapiens
tctccatcctccctggctgcatctgtcggagacagagtcgccatcacttgccgggcaagtcagagcatttccaactatgtaaattggtatcagcagaaaccagggaaagcccctaacctcctaatctctgctgcatccaatttacaaagtggggtcccatcaaggttcactggcagtggatctgggacagatttcactctcaccatcagtagtctggaacctgaagattttgcaacttactactgtcaacagagtcaggctgcccctctcactttcggcggagggaccaaggtggagatcaagcgaactgtggctgcacc

SEQ ID NO: 134 - protein, 106 aa, Homo sapiens
SPSSLAASVGDRVAITCRASQSISNYVNWYQQKPGKAPNLLISAASNLQSGVPSRFTGSGSGTDFTLTISSLEPEDFATYYCQQSQAAPLTFGGGTKVEIKRTVAA

SEQ ID NO: 135 - DNA, 320 bp, Homo sapiens
tctccatcctccctgggcgcatctgtaggggacatcgtcaccatcacttgccgggcaagtcagatcattaccacccatttaaattggtatcagcaaaaaccaggcaaagcccctaacctcctgatctatggtgcatccaatttgcaagctggggtcccatcgaggttcagtggcagtggatctgggactgatttcactctcaccatcagcagtctgcaaccagaagattttgcaacttactactgtcaccagacttacacgacccctctcactttcggcggagggaccaaggtggacatcaaacgaactgtggctgcacc

SEQ ID NO: 136 - protein, 106 aa, Homo sapiens
SPSSLGASVGDIVTITCRASQIITTHLNWYQQKPGKAPNLLIYGASNLQAGVPSRFSGSGSGTDFTLTISSLQPEDFATYYCHQTYTTPLTFGGGTKVDIKRTVAA

SEQ ID NO: 137 - DNA, 323 bp, Homo sapiens
tctccttcctccctgtctgcatctgtcggagacagagtcaccatctcttgccgggcaagtcagaccattaccagggccttaaactggtaccaacacacacctgggaaaggccctaaactcctgatctttggtgcatccagcttgcaaaggggggtctcatcgaggttcagtggcagtggctctgagacagatttcactctcaccatcagcggtctgcagcctgaagattttgcgacttactactgtcagcagactcagactagtcctcgctacacctttggccaagggaccaagctggagataaagcgaactgtggctgcacc

SEQ ID NO: 138 - protein, 107 aa, Homo sapiens
SPSSLSASVGDRVTISCRASQTITRALNWYQHTPGKGPKLLIFGASSLQRGVSSRFSGSGSETDFTLTISGLQPEDFATYYCQQTQTSPRYTFGQGTKLEIKRTVAA

SEQ ID NO: 139 - DNA, 323 bp, Homo sapiens
tctccatcctccctgtctgcatctgtaggagacagagtcaccatcacttgccgggccagtcagggcattgacaattatttagcctggtatcagcaaagaccagggaaagcccctaagctcctgatctatggtgcatccactttgcaaagtggggtcccatcaaggttcagcggcagtggatctgggacagtcttcactctcaccatcagcagcctgcagcctgaagattttgcaacttattactgtcaacagcttaatagttatccttcgatcaacttcggccaagggacacgactggagattaaacgaactgtggctgcacc

SEQ ID NO: 140 - protein, 107 aa, Homo sapiens
SPSSLSASVGDRVTITCRASQGIDNYLAWYQQRPGKAPKLLIYGASTLQSGVPSRFSGSGSGTVFTLTISSLQPEDFATYYCQQLNSYPSINFGQGTRLEIKRTVAA

SEQ ID NO: 141 - DNA, 320 bp, Homo sapiens
tctccatcctccctgtctgcatctgtaggagacagagtcaccatcacttgccaggcgagtcaggacattagttactatttaaattggtatcagcagaaaccagggaaagcccctaagctcctgatctacgatgcattgaatgtggaaacaggggtcccatcaaggttcggtggaagtggatctgggacagatttcactttcaccatcagcagcctgcagcctggagattttgcaatatattactgtcagcagtatgctaatttcccgtatacttttggccaggggaccaagctggagatcaaacgaactgtggctgcacc

SEQ ID NO: 142 - protein, 106 aa, Homo sapiens
SPSSLSASVGDRVTITCQASQDISYYLNWYQQKPGKAPKLLIYDALNVETGVPSRFGGSGSGTDFTFTISSLQPGDFAIYYCQQYANFPYTFGQGTKLEIKRTVAA

SEQ ID NO: 143 - DNA, 320 bp, Homo sapiens
tctccatcctccctgtctgtatctgtaggagacagagtcaccatcacttgccaggcgagtcacgacattagcaactatttacattggtttcagcagaaaccaggggaagcccctaagctcctgatctacgatgcatccaatttggaaacaggggtcccatcaaggttcaggggaagtggatttgggacagattttactttcaccatcagcagcctgcagcctgaagatattgcaacatattactgtcaacagtatggtaatctcccgtacacttttggccaggggaccaagctgcagatcaaacgaactgtggctgcacc

SEQ ID NO: 144 - protein, 106 aa, Homo sapiens
SPSSLSVSVGDRVTITCQASHDISNYLHWFQQKPGEAPKLLIYDASNLETGVPSRFRGSGFGTDFTFTISSLQPEDIATYYCQQYGNLPYTFGQGTKLQIKRTVAA

SEQ ID NO: 145 - DNA, 320 bp, Homo sapiens
tctccatcctccctgtccgcatctataggagacagagtcagtatcgcttgccaggcgagtgagggcattagcaactatttaaattggtatcagcagaaaccagggaaagcccctaagctcctaatctacgatgcatccaatttggaatcaggggtcccatcaagatttagtggaagtggccttgagacagattttactctcaccatcaacagcctgcagcctgaagatattgcaacatattactgtcaacagtatgatagtctccctctcactttcggcggagggaccaaggtggagatcaaacgaactgtggctgcacc

SEQ ID NO: 146 - protein, 106 aa, Homo sapiens
SPSSLSASIGDRVSIACQASEGISNYLNWYQQKPGKAPKLLIYDASNLESGVPSRFSGSGLETDFTLTINSLQPEDIATYYCQQYDSLPLTFGGGTKVEIKRTVAA

SEQ ID NO: 147 - DNA, 320 bp, Homo sapiens
tctccatcctccctgtctgcatctgtaggagacagagtcaccatcacttgccaggcgagtcaggacattagcaactatttaaattggtatcaacagaaaccagggaaagcccctaagctcctgatctacgatgcatccaatttgcaagcaggggtcccgtcaaggttcagtggaagtggatctgggacagattttactttcaccatcagcagcctgcagcctgaagatattgcaacatattactgtcaacagtatgataatctccctcccactttcggcggagggaccaaggtggagttcaaacgaactgtggctgcacc

SEQ ID NO: 148 - protein, 106 aa, Homo sapiens
SPSSLSASVGDRVTITCQASQDISNYLNWYQQKPGKAPKLLIYDASNLQAGVPSRFSGSGSGTDFTFTISSLQPEDIATYYCQQYDNLPPTFGGGTKVEFKRTVAA

SEQ ID NO: 149 - DNA, 320 bp, Homo sapiens
tctccatcctccctgtctgcatctgtaggagacagagtcaccatcacttgtcgggcgagtcagggcattaataattatttagcctggtatcagcagaaaccagggaaagttccgcagctcctgatcgatgctgcatccactttgcaatcaggggtcccatctcggttcagtggcagtggatctgggacagatttcactctcaccatcagcagcctgcagcctgaagatgttgcaacttattactgtcaaaagtataacagtgccccattcactttcggccctgggaccaaagtggatatcaaacgaactgtggctgcacc

SEQ ID NO: 150 - protein, 106 aa, Homo sapiens
SPSSLSASVGDRVTITCRASQGINNYLAWYQQKPGKVPQLLIDAASTLQSGVPSRFSGSGSGTDFTLTISSLQPEDVATYYCQKYNSAPFTFGPGTKVDIKRTVAA

SEQ ID NO: 151 - DNA, 323 bp, Homo sapiens
tctccagccaccctgtctgtgtcgccaggggaaagagccaccctctcctgcagggccagtcagagtattagcagcaacttagcctggtaccagcagagacctggccaggctcccaggctcctcatctatgatgcatccaccagggccactggtgtcccagccaggttcagtggcagtgggtctgggacagagtacactctcaccatcagcagcctgcagtctgaagattttgcagtttattactgtcagcagtataagaactggcctccgtactcttttggccaggggaccaagctggacataaaacgaactgtggctgcacc

SEQ ID NO: 152 - protein, 107 aa, Homo sapiens
SPATLSVSPGERATLSCRASQSISSNLAWYQQRPGQAPRLLIYDASTRATGVPARFSGSGSGTEYTLTISSLQSEDFAVYYCQQYKNWPPYSFGQGTKLDIKRTVAA

SEQ ID NO: 153 - DNA, 317 bp, Homo sapiens
tctccagccaccctgtctgtgtctccaggggaaagagccaccctctcctgcagggccagtcagagtgttagcagcaacttagcctggtaccagcagaaagttggccaggctcccaggctcctcatctatggtgcatccagcagggccactggtatcccagccaggttcagtggcactgggtctgggacagagttcactctcaccatcagcagcctgcagtctgaagattttgcagtttattactgtcagcagtataatgactggtacacttttggccaggggaccaagctggagatcaaacgaactgtggctgcacc

SEQ ID NO: 154 - protein, 105 aa, Homo sapiens
SPATLSVSPGERATLSCRASQSVSSNLAWYQQKVGQAPRLLIYGASSRATGIPARFSGTGSGTEFTLTISSLQSEDFAVYYCQQYNDWYTFGQGTKLEIKRTVAA

SEQ ID NO: 155 - DNA, 323 bp, Homo sapiens
tctccaggcaccctgtctgtgtctccagggggaagagccaccctctcctgcagggccagtcagagtgttggcagcaacttagtctggtatcaacataaacctggccaggctcccagactcctcatctatggtgcatccaccagggccactggtatcccagccaggtttagtggcagtgggtctgggacagagttcactctcaccatcagcggcctgcagtctgaagattttgcaatttattactgtcagcagtataataattggcctcgcgggacgttcggccaagggaccaaggtggaagtcaaacgaactgtggctgcacc

SEQ ID NO: 156 - protein, 107 aa, Homo sapiens
SPGTLSVSPGGRATLSCRASQSVGSNLVWYQHKPGQAPRLLIYGASTRATGIPARFSGSGSGTEFTLTISGLQSEDFAIYYCQQYNNWPRGTFGQGTKVEVKRTVAA

SEQ ID NO: 157 - DNA, 323 bp, Homo sapiens
tctccaggcaccctgtctgtgtctccaggggagagagccaccctctcctgcagggccagtcagaatattgacagcttcttagcctggtaccagcagaaacctggccaggctcccaggctcctcatttatggtgcgtccaccagggccactggtatcccagccaggttcagtggcagtgggtctgggacagaattcactctcaccattagcagcctgcagtctgaagactttgcagtttattactgtcaacagtatgataactggcctccggccacttttggccaggggaccaagctggagatgaaacgaactgtggctgcacc

SEQ ID NO: 158 - protein, 107 aa, Homo sapiens
SPGTLSVSPGERATLSCRASQNIDSFLAWYQQKPGQAPRLLIYGASTRATGIPARFSGSGSGTEFTLTISSLQSEDFAVYYCQQYDNWPPATFGQGTKLEMKRTVAA

SEQ ID NO: 159 - DNA, 323 bp, Homo sapiens
tctccagccaccctgtctgtgtctccaggggacagagtcaccctctcctgcagggccagtcacagtgttagtagcaacttagcctggtaccagcagaaacctggccaggctcccaggctcctcgtctatgatgcatccaccagggccactgatgtcccagccaggttcagtggcagtgggtctgggacagaattcactctcaccatcagcagcctgcagtctgaagattttgcagtttattactgtcagcagtataataactggcctctgtggacgctcggccaagggaccaaggtggaaatcaaacgaactgtggctgcacc

SEQ ID NO: 160 - protein, 107 aa, Homo sapiens
SPATLSVSPGDRVTLSCRASHSVSSNLAWYQQKPGQAPRLLVYDASTRATDVPARFSGSGSGTEFTLTISSLQSEDFAVYYCQQYNNWPLWTLGQGTKVEIKRTVAA

SEQ ID NO: 161 - DNA, 323 bp, Homo sapiens
tctccaggcaccctgtctttgtctccaggggaaagagccaccctctcctgcagggccagtcagactgttttcagccgctacttagcctggtaccagcagaaacctgggcggtctcccaggctcctcatctatgttgcatccagcagggccactggcatcccagacaggttcagtggcagtgggtctgggacagacttcactctcaccatcagcagactggagcctgaagattttgcagtgtattactgtcagcaatatggtagctcaccgtacacttttggccaggggaccaaggtggagatcaaacgaactgtggctgcacc

SEQ ID NO: 162 - protein, 107 aa, Homo sapiens
SPGTLSLSPGERATLSCRASQTVFSRYLAWYQQKPGRSPRLLIYVASSRATGIPDRFSGSGSGTDFTLTISRLEPEDFAVYYCQQYGSSPYTFGQGTKVEIKRTVAA

SEQ ID NO: 163 - DNA, 323 bp, Homo sapiens
tctccaggcaccctgtccttgtctccaggggaaagagccaccctctcctgcagggccagtcagagtgttagtagtaggtacttagcctggtaccagcagaaacctggccaggctcccaggctcctcatctatggtgcatccagcagggccactggcatcccagacaggttcagtggcagtgggtctgggacagacttcactctcaccatcaacagactggagcctgaagattttgcagtgtattactgtcagcagtatggtacctcaccgtacacttttggccaggggaccaagctggagatcaaacgaactgtggctgcacc

SEQ ID NO: 164 - protein, 107 aa, Homo sapiens
SPGTLSLSPGERATLSCRASQSVSSRYLAWYQQKPGQAPRLLIYGASSRATGIPDRFSGSGSGTDFTLTINRLEPEDFAVYYCQQYGTSPYTFGQGTKLEIKRTVAA

SEQ ID NO: 165 - DNA, 326 bp, Homo sapiens
tctccaggcaccctgtctttgtctccaggggaaagagccaccctctcctgcagggccagtgagagcataagcagcagctacttagcctggtaccagcagaaacctggccaggctcccaggctcctcatctatggtgcatccaacagggcctctggcatcccagacaggttcattggcagtgggtctgcgacagacttcactctcaccatcagcagactggagcctgaagattttgcagtgtattactgtcagcgttatgatacctcactccggaggacgttcggccaagggaccaaggtggaaatcaaacgaactgtggctgcacc

SEQ ID NO: 166 - protein, 108 aa, Homo sapiens
SPGTLSLSPGERATLSCRASESISSSYLAWYQQKPGQAPRLLIYGASNRASGIPDRFIGSGSATDFTLTISRLEPEDFAVYYCQRYDTSLRRTFGQGTKVEIKRTVAA

SEQ ID NO: 167 - DNA, 323 bp, Homo sapiens
tctccaggcagcctgtctttgtctccaggggagagagccaccctctcttgcagggccagtcagagtgtgaggagcaactacgtagcctggtaccagaaggaacctggccgggctccccgactcctcatctatggtgcatccaccagggccagtggcatcccagacaggttcagtggcagtgggtctggggcagacttcactctcaccatcagtggactggagcctgaagattttgcagtgtattactgtcagcagtatggtagctcaccggggacttttggccaggggacgaggctggagatcaaacgaactgtggctgcacc

SEQ ID NO: 168 - protein, 107 aa, Homo sapiens
SPGSLSLSPGERATLSCRASQSVRSNYVAWYQKEPGRAPRLLIYGASTRASGIPDRFSGSGSGADFTLTISGLEPEDFAVYYCQQYGSSPGTFGQGTRLEIKRTVAA

SEQ ID NO: 169 - DNA, 338 bp, Homo sapiens
tctccagactccctgcctgtgtctctgggcgagagggccaccatcaactgcaagtccagccagactattttatatggctccagtaataagaattccttggcttggtaccagcagaaaccaggacagcctcctaggctgctcatttcctgggcatctacccgggaatccggggtccctgaccgattcagtggcagcgggtctgggacagatttcactctcaccatcagtggcctgcaggctgaggatgtggcagtttattactgtcaacaatatcattctgttccgtggacgttcggccaggggaccaaggtggaattcaaacgaactgtggctgcacc

SEQ ID NO: 170 - protein, 112 aa, Homo sapiens
SPDSLPVSLGERATINCKSSQTILYGSSNKNSLAWYQQKPGQPPRLLISWASTRESGVPDRFSGSGSGTDFTLTISGLQAEDVAVYYCQQYHSVPWTFGQGTKVEFKRTVAA

SEQ ID NO: 171 - DNA, 338 bp, Homo sapiens
tctccagactccctgcctgtgtctctgggcgagagggccaccatcaactgcaagtccagccagactattttatacggctccagtaataagaattccttggcttggtaccagcagaaaccaggacagcctcctaggctgctcatttcctgggcatctacccgggaatccggggtccctgaccgattcagtggcagcgggtctgggacagatttcactctcaccatcagtggcctgcaggctgaggatgtggcagtttattactgtcaacaatatcattctgttccgtggacgttcggccaggggaccaaggtggaattcaaacgaactgtggctgcacc

SEQ ID NO: 172 - protein, 112 aa, Homo sapiens; identical to SEQ ID NO: 170

SEQ ID NO: 173 - DNA, 338 bp, Homo sapiens
tctccagactccctgcctgtgtctctgggcgagagggccaccatcaactgcaagtccagccagactattttatacggctccagtaataagaattcctkggcttggtaccagcagaaaccaggacagcctcctaggttgctcatttcctgggcctctacccgggaatccggggtccctgaccgattcagtggcagcgggtctgggacagatttcactctcaccatcagtggcctgcaggctgaagatgtggcagtttattactgtcaacaatatcattctgttccgtggacgttcggccaggggaccaaggtggaattcaaacgaactgtggctgcacc

SEQ ID NO: 174 - protein, 112 aa, Homo sapiens; identical to SEQ ID NO: 170

SEQ ID NO: 175 - DNA, 338 bp, Homo sapiens
tctccagactccctgcctgtgtctctgggcgagagggccaccatcaactgcaagtccagccagactattttatacggctccagtaataagaattccttggcttggtaccagcagaaaccaggacagcctcctaggctgctcatttcctgggcatctacccgggaatccggggtccctgaccgattcagtggcagcgggtctgggacagatttcactctcaccatcagtggcctgcaggctgaagatgtggcagtttattactgtcaacaatatcattccattccgtggacgttcggccaggggaccaaggtggacttcaaacgaactgtggctgcacc

SEQ ID NO: 176 - protein, 112 aa, Homo sapiens
SPDSLPVSLGERATINCKSSQTILYGSSNKNSLAWYQQKPGQPPRLLISWASTRESGVPDRFSGSGSGTDFTLTISGLQAEDVAVYYCQQYHSIPWTFGQGTKVDFKRTVAA

SEQ ID NO: 177 - DNA, 341 bp, Homo sapiens
tctccagactccctggctgtgtctctgggcgagagggccaccatcaactgcaagtccagccagagtgttttatacagctccaacaataagaacttcttagcttggtaccagcagaaaccaggacagcctcctaaactgctcatttactgggcatctacccgggaatccggggtccctgaccgattcagtggcagcgggtctgggacagagttcactctcaccatcagcaacctgcaggctgaagatgtggcagattattactgtcagcaatattatggtagtcctccgtggacgttcggccaagggaccagggtggaaatcaaacgaactgtggctgcacc

SEQ ID NO: 178 - protein, 113 aa, Homo sapiens
SPDSLAVSLGERATINCKSSQSVLYSSNNKNFLAWYQQKPGQPPKLLIYWASTRESGVPDRFSGSGSGTEFTLTISNLQAEDVADYYCQQYYGSPPWTFGQGTRVEIKRTVAA

SEQ ID NO: 179 - DNA, 338 bp, Homo sapiens
tctccagactccctggctgtgtctctgggcgagagggccaccatcaactgcaagtccagtcagagtgttttatacatctccaacaataagaactgcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagtattatagtactccttggacgttcggccaagggaccaaggtggaaatcaaacgaactgtggctgcacc

SEQ ID NO: 180 - protein, 112 aa, Homo sapiens
SPDSLAVSLGERATINCKSSQSVLYISNNKNCLAWYQQKPGQPPKLLIYWASTRESGVPDRFSGSGSGTDFTLTISSLQAEDVAVYYCQQYYSTPWTFGQGTKVEIKRTVAA

SEQ ID NO: 181 - DNA, 338 bp, Homo sapiens
tctccagactccctggctgtgtctctgggcgagagggccaccatcaactgcaagtccagccagcgtgttttccacaactccaacaataaaaactggttggcttggtaccagcagaaaccgggacaacctcctaagctgctcatttactgggcatctacacgggaatccggggtccctgaccgattcagtggcagcgggtctgggacagatttcactctcaccatcagcagcctgcaggctgaagatgtggcagtttattactgtcagcagtattatagtgctccccggacgttcggccaagggaccaaggtggaaatcaaacgaactgtggctgcacc

SEQ ID NO: 182 - protein, 112 aa, Homo sapiens
SPDSLAVSLGERATINCKSSQRVFHNSNNKNWLAWYQQKPGQPPKLLIYWASTRESGVPDRFSGSGSGTDFTLTISSLQAEDVAVYYCQQYYSAPRTFGQGTKVEIKRTVAA

SEQ ID NO: 183 - DNA, 338 bp, Homo sapiens
tctccagactccctggctgtgtctctgggcgagagggccaccatcaactgcaagtccagccagagtgttttatacagctccagcaatgagaactacttagctkggtaccaacagaagccagggcagcctcctaaactgctcatttactgggcatctacccgggaatccggggtccctgaccgattcaatgtcagcaggtctgggacagatttcactctcaccatcagcagcctgcaggctgcagatgtggcagtttattactgtcaccaatattacagtactccgtacacttttggccaggggaccaggctggagatcaagcgaactgtggctgcacc

SEQ ID NO: 184 - protein, 112 aa, Homo sapiens
SPDSLAVSLGERATINCKSSQSVLYSSSNENYLAWYQQKPGQPPKLLIYWASTRESGVPDRFNVSRSGTDFTLTISSLQAADVAVYYCHQYYSTPYTFGQGTRLEIKRTVAA

SEQ ID NO: 185 - DNA, 338 bp, Homo sapiens
tctccagactccctggctgtgtctctgggcgagagggccaccatcaactgcaagtccagccagagtgttttatacagctccagcaatgagaactacttagcttggtaccaacagaagccagggcagcctcctaaactgctcatttactgggcatctacccgggaatccggggtccctgaccgattcagtggcagcgggtctgggacagatttcactctcaccatcagcagcctgcaggctgcagatgtggcagtttattactgtcaccaatattacagtactccgtacacttttggccaggggaccaggctggagatcaagsgaactgtggctgcacc

SEQ ID NO: 186 - protein, 112 aa, Homo sapiens
SPDSLAVSLGERATINCKSSQSVLYSSSNENYLAWYQQKPGQPPKLLIYWASTRESGVPDRFSGSGSGTDFTLTISSLQAADVAVYYCHQYYSTPYTFGQGTRLEIKRTVAA

SEQ ID NO: 187 - DNA, 338 bp, Homo sapiens
tctccagactccctggctgtgtctctgggcgagagggccaccatcaactgcaagtccagccagagtgttttatacaactccaacaataagaactacttagcttggtaccagcagagaccaggacagcctcctaagctgctcattcgctgggcatctacccgggactccggggtccctgaccgattcagtggcagcgggtctgggacagatttcactctcaccatcagcagcctgcaggctgaggatgtggcagtttattactgtcagcaatattatagtagtcctcccactttcggccctgggaccaaagtggatatcaaacgaacggtggctgcacc

SEQ ID NO: 188 - protein, 112 aa, Homo sapiens
SPDSLAVSLGERATINCKSSQSVLYNSNNKNYLAWYQQRPGQPPKLLIRWASTRDSGVPDRFSGSGSGTDFTLTISSLQAEDVAVYYCQQYYSSPPTFGPGTKVDIKRTVAA

SEQ ID NO: 189 - DNA, 338 bp, Homo sapiens
tctccagactccctggctgtgtctctgggcgagagggccaccatcaactgcaggtccagccagagtctttcatccacctccgacaataacaaccacttaagttggtaccaggtgaaaccaggacagtctcctagactgctcatttactgggcatctaaccgggaatcaggggtccctgaccgattcagcggcagcgggtctgggacagatttcactctcaccatcagcagcctgcaggctgaagatgtggcactctattactgtctacactattctaatactttttggacattcggccaagggaccagggtggaaatcaaacgaactgtggctgcacc

SEQ ID NO: 190 - protein, 112 aa, Homo sapiens
SPDSLAVSLGERATINCRSSQSLSSTSDNNNHLSWYQVKPGQSPRLLIYWASNRESGVPDRFSGSGSGTDFTLTISSLQAEDVALYYCLHYSNTFWTFGQGTRVEIKRTVAA

SEQ ID NO: 191 - DNA, 454 bp, Homo sapiens
caggtgaaactgctcgagtctgggggaggtgtggtacagccgggggggtccctgagactctcctgtgcagcctctggattcagctttagcagctataccatgtcctgggtccgccaggctccagggaaggggctggagtgggtctcaggtattagtggtagtgggagcgcatactacggagactccgtgaagggccggtttaccatctccagagacaattccaagaacacgctgtatctgcaaatgaacagcctgagagccgaggacacggccgtatattactgtgcgaaagcctccgcccagggggtagtggttctctccgcgggatttcgatactactttaactactggggccagggaaccctggtcaccgtctcctcagcttccaccaagggcccatcggtcttccccctggcgccctgctccaggagcacctctgggggcacagcggccc

SEQ ID NO: 192 - protein, 151 aa, Homo sapiens
QVKLLESGGGVVQPGGSLRLSCAASGFSFSSYTMSWVRQAPGKGLEWVSGISGSGSAYYGDSVKGRFTISRDNSKNTLYLQMNSLRAEDTAVYYCAKASAQGVVVLSAGFRYYFNYWGQGTLVTVSSASTKGPSVFPLAPCSRSTSGGTAA

SEQ ID NO: 193 - DNA, 457 bp, Homo sapiens
caggtgaaactgctcgagtctgggggaggcttggtacagcctggggggtccctgagactctcctgtgcagcctctggattcacctttagcagctatgccatgagctgggtccgccaggctccagggaaggggctggagtgggtctcagcttttagtggtagtggaggtggcacatactacgcagactccgtgaagggccggttcaccatctccagagacaattccaagaacacgctgtatctgcaaatgaacagcctgagagccgaggacacggccgtatattactgtgcgaaagcctccgcccagggggtagtggttctctccgcgggatttcgatactactttaactactggggccagggaaccctggtcaccgtctcctcagcttccaccaagggcccatcggtcttccccctggcgccctgctccaggagcacctctgggggcacagcggccc

SEQ ID NO: 194 - protein, 152 aa, Homo sapiens
QVKLLESGGGLVQPGGSLRLSCAASGFTFSSYAMSWVRQAPGKGLEWVSAFSGSGGGTYYADSVKGRFTISRDNSKNTLYLQMNSLRAEDTAVYYCAKASAQGVVVLSAGFRYYFNYWGQGTLVTVSSASTKGPSVFPLAPCSRSTSGGTAA

SEQ ID NO: 195 - DNA, 460 bp, Homo sapiens
caggtgaaactgctcgagtctgggggaggcttggtacagcctggggggtccctgagactctcctgtgcagcctctggattcacctttagcagctataccatgtcctgggtccgccaggctccagggaaggggctggagtgggtctcagcttttagtggtagtggaggtggcacatactacgcagactccgtgaagggccggttcaccatctccagagacaattccaagaacacgctgtatctgcaaatgaacagcctgagagccgaggacacggccgtatattactgtgcgaaagccgcctccgcccagggggttctcgttctctccgcgggatttcgatactactttaactactggggccagggaaccctggtcaccgtctcctcagcttccaccaagggcccatcggtcttccccctggcgccctgctccaggagcacctctgggggcacagcggccc

SEQ ID NO: 196 - protein, 153 aa, Homo sapiens
QVKLLESGGGLVQPGGSLRLSCAASGFTFSSYTMSWVRQAPGKGLEWVSAFSGSGGGTYYADSVKGRFTISRDNSKNTLYLQMNSLRAEDTAVYYCAKAASAQGVLVLSAGFRYYFNYWGQGTLVTVSSASTKGPSVFPLAPCSRSTSGGTAA

SEQ ID NO: 197 - DNA, 451 bp, Homo sapiens
caggtgaaactgctcgagtctgggggaggcttggtacagcctggggggtccctgagactctcctgtgcagcctctggattcacctttagcagctatgccatgagctgggtccgccaggctccagggaaggggctggagtgggtctcagcttttagtggtagtggaggtggcacatactacgcagactccgtgaagggccggttcaccatctccagagacaattccaagaacacgctgtatctgcaaatgaacagcctgagagccgaggacacggccgtatattactgtgcgaaagcctccgcggtagtggttctctccgcgggatttcgatactactttaactactggggccagggaaccctggtcaccgtctcctcagcttccaccaagggcccatcggtcttccccctggcgccctgctccaggagcacctctgggggcacagcggccc

SEQ ID NO: 198 - protein, 150 aa, Homo sapiens
QVKLLESGGGLVQPGGSLRLSCAASGFTFSSYAMSWVRQAPGKGLEWVSAFSGSGGGTYYADSVKGRFTISRDNSKNTLYLQMNSLRAEDTAVYYCAKASAVVVLSAGFRYYFNYWGQGTLVTVSSASTKGPSVFPLAPCSRSTSGGTAA

SEQ ID NO: 199 - DNA, 442 bp, Homo sapiens
caggtgaaactgctcgagtctgggggaggcttggtacagcctggggggtccctgagactctcctgtgcagcctctggattcacctttagcagctatgccatgagctgggtccgccaggctccagggaaggggctggagtgggtctcagcttttagtggtagtggaggtggcacatactacgcagactccgtgaagggccggttcaccatctccagagacaattccaagaacacggtgtatctgcaaatgaacagcctgagacctgaggacacggctatgtattactgtgtgaaagatcacgtagcagtgcctggttttctctcttactttgaccactggggccagggaaccctggtcaccgtctcctcagcttccaccaagggcccatcggtcttccccctggcgccctgctccaggagcacctctgggggcacagcggccc

SEQ ID NO: 200 - protein, 147 aa, Homo sapiens
QVKLLESGGGLVQPGGSLRLSCAASGFTFSSYAMSWVRQAPGKGLEWVSAFSGSGGGTYYADSVKGRFTISRDNSKNTVYLQMNSLRPEDTAMYYCVKDHVAVPGFLSYFDHWGQGTLVTVSSASTKGPSVFPLAPCSRSTSGGTAA

SEQ ID NO: 201 - DNA, 442 bp, Homo sapiens
caggtgaaactgctcgagtctgggggaggcttggtacagcctggggggtccctgagactctcctgtgcagcctctggattcacctttagcagctatgccatgagctgggtccgccaggctccagggaaggggctggagtgggtctcagcttttagtggtagtggaggtggcacatactacgcagactccgtgaagggccgattcaccatttccagagacaattccaagaacacggtgtatctgcaaatgaacagcctgagacctgaggacacggctatgtattactgtgtgaaagatcacgtagcagtgcctggttttctctcttactttgaccactggggccagggaaccctggtcaccgtctcctcagcctccaccaagggcccatcggtcttccccctggcgccctgctccaggagcacctctgggggcacagcggccc

SEQ ID NO: 202 - protein, 147 aa, Homo sapiens; identical to SEQ ID NO: 200

SEQ ID NO: 203 - DNA, 436 bp, Homo sapiens
caggtgaaactgctcgagtctgggggaggcgtggtccagcctgggaggtcccttagactctcctgtgcagcgtctggattcatcttcagtagttatggcatgcattgggtccgccaggctccaggcaaggggctggagtgggtggcgtttataccatttgatggaaagaacaaatactatggagactctgtgaagggccgattcaccatctccagagacaattccgagaacacgctgtatctgcagatgaacagcctgagaactgatgacacggctgtgtattactgtgcgaaagaccgcattgagagattaatgtctggtcttgactactggggccagggatccctggtcaccgtctcctcagcctccaccaagggcccatcggtcttccccctggcaccctcctccaagagcacctctgggggcacagcggccc

SEQ ID NO: 204 - protein, 145 aa, Homo sapiens
QVKLLESGGGVVQPGRSLRLSCAASGFIFSSYGMHWVRQAPGKGLEWVAFIPFDGKNKYYGDSVKGRFTISRDNSENTLYLQMNSLRTDDTAVYYCAKDRIERLMSGLDYWGQGSLVTVSSASTKGPSVFPLAPSSKSTSGGTAA

SEQ ID NO: 205 - DNA, 448 bp, Homo sapiens
caggtgaaactgctcgagtctggcccaggactggtggagccttcacagaccctgaccctcacctgctctgtctctggcggctccatcaccagtgatagttactactggggctggatccgccagcacccagggaagggcctggagtggattgggtacatcgttcacagtgggatcgcctactacaacccggccctcaagggtcgagctaccatatcactagacacctctaagaaccgggtgtccctgaagctgagctctgcgacggccgcggacacggccgtgtattactgtgcgagagatagtagccgtaaggatcgaggcttcagagcctggttcgacccctggggccagggaaccctggtcaccgtctcctcagcctccaccaagggcccatcggtcttccccctggcgccctgctccaggagcacctctgggggcacagcggccc

SEQ ID NO: 206 - protein, 149 aa, Homo sapiens
QVKLLESGPGLVEPSQTLTLTCSVSGGSITSDSYYWGWIRQHPGKGLEWIGYIVHSGIAYYNPALKGRATISLDTSKNRVSLKLSSATAADTAVYYCARDSSRKDRGFRAWFDPWGQGTLVTVSSASTKGPSVFPLAPCSRSTSGGTAA

SEQ ID NO: 207 - DNA, 451 bp, Homo sapiens
caggtgaaactgctcgagtctggcccaggactggtgaagccttcggagaccctgtccctcacctgcaccgtctctggtggctccatcaatacttatatctggcactggatccggcagtccccagggaagggactggagtggattggtcacatctattacagtgggagctccttcgccaacccgtccctcaagagtcgcatttccatttcagtggccgcctctaagaaccagttcttcctcgatctgaactctgtgacggccgcggacacggccgtctattactgtgcgagagaacgaattctggctagtggctatgggagggactacaactccgggatggacgtctggggccagggaaccctggtcaccgtctcctcagcctccaccaagggcccatcggtcttccccctggcaccctcctccaagagcacctctgggggcacagcggccc

SEQ ID NO: 208 - protein, 150 aa, Homo sapiens
QVKLLESGPGLVKPSETLSLTCTVSGGSINTYIWHWIRQSPGKGLEWIGHIYYSGSSFANPSLKSRISISVAASKNQFFLDLNSVTAADTAVYYCARERILASGYGRDYNSGMDVWGQGTLVTVSSASTKGPSVFPLAPSSKSTSGGTAA

SEQ ID NO: 209 - DNA, 433 bp, Homo sapiens
caggtgaaactgctcgagtctggggcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacccttggatacacgtggcccacactttgactactggggccagggaaccctggtcaccgtctcctcagcttccaccaagggcccatcggtcttccccctggcgccctgctccaggagcacctctgggggcacagcggccc

SEQ ID NO: 210 - protein, 144 aa, Homo sapiens
QVKLLESGAEVKKPGESLKISCKGSGYSFTSYWIGWVRQMPGKGLEWMGIIYPGDSDTRYSPSFQGQVTISADKSISTAYLQWSSLKASDTAMYYCARPLDTRGPHFDYWGQGTLVTVSSASTKGPSVFPLAPCSRSTSGGTAA

SEQ ID NO: 211 - DNA, 317 bp, Homo sapiens
tctccatcctccctgtctgcatctgtaggagacagagtcaccatcacttgccgggcaagtcagaacattaggaagtatttaaattggtatcagcacaaaccagggaaagcccctaaactcctgatctttcttgcatccaatttgcaaagtggagtcgcatccagattcagtggcagtggatctggggctgatttcagtctcaccatcagcagtctgcaacctgaagattttgcaacttactactgtcaagcttataacaatatccctactttcggccctgggaccaaagtggatatcagacgaactgtggctgcacc

SEQ ID NO: 212 - protein, 105 aa, Homo sapiens
SPSSLSASVGDRVTITCRASQNIRKYLNWYQHKPGKAPKLLIFLASNLQSGVASRFSGSGSGADFSLTISSLQPEDFATYYCQAYNNIPTFGPGTKVDIRRTVAA

SEQ ID NO: 213 - DNA, 317 bp, Homo sapiens
tctccatcctccctgtctgcatctgtaggagacagagtcaccatcacttgccgggcaagtcagaacattaggaagtatttaaattggtatcagcacaaaccagggaaagcccctaaactcctgatctttcttgcatccaatttgcaaaatggagtcgcatccagattcagtggcagtggatctggggctgatttcagtctcaccatcagcagtctgcaacctgaagattttgcaacttactactgtcaagcttataacaatatccctactttcggccctgggaccaaagtggatatcagacgaactgtggctgcacc

SEQ ID NO: 214 - protein, 105 aa, Homo sapiens
SPSSLSASVGDRVTITCRASQNIRKYLNWYQHKPGKAPKLLIFLASNLQNGVASRFSGSGSGADFSLTISSLQPEDFATYYCQAYNNIPTFGPGTKVDIRRTVAA

SEQ ID NO: 215 - DNA, 317 bp, Homo sapiens
tctccatcctccctgtctgcatctgtaggagacagagtcaccatcacttgccgggcaagtcagaacattaggaagtatttaaattggtatcagcacaaaccagggaaagcccctaaactcctgatctttcttgcatccaatttgcaaagtggagtcgcatccagattcagtggcagtggatctggggctgatttcagtctcaccatcagcagtctgcaacctgaagattttgcaacttactactgtcaagcttataacaatatccctactttcggccccgggaccaaagtggatatcagacgaactgtggctgcacc

SEQ ID NO: 216 - protein, 105 aa, Homo sapiens; identical to SEQ ID NO: 212

SEQ ID NO: 217 - DNA, 320 bp, Homo sapiens
tctccatcctccctatctgcatctgtaggagacagagtcaccatcacctgccgggcaagtcagaccattggcacctatttaaattggtatcagcacaaaccagggaaagcccctaagctcctgatctatgttgcatccagtttgcaaagtggggtcccatcaaggttcagtggcagtggatctgggacagatttcactctcaccatcagcagtctgcaacctgaagattttgcagcttactactgtcaacagagttacggtacccctccaacttttggccaggggaccaagctggagatcaaacgaactgtggctgcacc

SEQ ID NO: 218 - protein, 106 aa, Homo sapiens
SPSSLSASVGDRVTITCRASQTIGTYLNWYQHKPGKAPKLLIYVASSLQSGVPSRFSGSGSGTDFTLTISSLQPEDFAAYYCQQSYGTPPTFGQGTKLEIKRTVAA

SEQ ID NO: 219 - DNA, 320 bp, Homo sapiens
tctccatcctccctatctgcatctgtaggagacagagtcaccatcacctgccgggcaagtcagaccattggcacctatttaaactggtatcagcacaaaccagggaaagcccctaagctcctgatctatgttgcatccagtttgcaaagtggggtcccatcaaggttcagtggcagtggatctgggacagatttcactctcaccatcagcagtctgcaacctgaagattttgcagcttactactgtcaacagagttacggtacccctccaacttttggccaggggaccaagctggagatcaaacgaactgtggctgcacc

SEQ ID NO: 220 - protein, 106 aa, Homo sapiens; identical to SEQ ID NO: 218

SEQ ID NO: 221 - DNA, 320 bp, Homo sapiens; identical to SEQ ID NO: 217

SEQ ID NO: 222 - protein, 106 aa, Homo sapiens; identical to SEQ ID NO: 218

SEQ ID NO: 223 - DNA, 320 bp, Homo sapiens
tctccatcctccctgtctgcatctgtaggagacagagtcaccatcacttgccgggcaagtcagagcattagaaattttctaaattggtatcagcagacaccagggaaagcccctaagctcctgatctatgctgcatccagtctgcaaagtggggtcccatcaaagttcagtggcagtggatctgggacagagttcactctcaccatcagcagtctgcaacctgaagattttgccacttattactgtcaacagagttacagtacccctctgacgttcggccaagggaccaaggtggaaatcaaacgaactgtggctgcacc

SEQ ID NO: 224 - protein, 106 aa, Homo sapiens
SPSSLSASVGDRVTITCRASQSIRNFLNWYQQTPGKAPKLLIYAASSLQSGVPSKFSGSGSGTEFTLTISSLQPEDFATYYCQQSYSTPLTFGQGTKVEIKRTVAA

SEQ ID NO: 225 - DNA, 320 bp, Homo sapiens
tctccatcctccctgtctgcatctgtaggagacagagtcaccatcacttgccgggcaagtcagagcattggcggctatttaaattggtatcagcagaaaccagggaaagcccctaacctcctgatctatactgcatccagtttgcaaagtggggtcccatcaaggttcagtggcagtggatctgggacagatttcactctcagcatcagcagtctgcaacctgaagattttgcaacttactactgtcaacagagttacactacccctaggacgttcggccaagggaccaaggtggaaatcaaacgaactgtggctgcacc

SEQ ID NO: 226 - protein, 106 aa, Homo sapiens
SPSSLSASVGDRVTITCRASQSIGGYLNWYQQKPGKAPNLLIYTASSLQSGVPSRFSGSGSGTDFTLSISSLQPEDFATYYCQQSYTTPRTFGQGTKVEIKRTVAA

SEQ ID NO: 227 - DNA, 320 bp, Homo sapiens
tctccatcctccctgtctgcatctgtaggagacacagtcaccatcacttgccgggcaagtcagaacatttactactatttatattggtatcagcagaaaccaggaaaagcccctaatctcctgatatatggtgcatccagtttgcaaagtggggtcccatcaaggttcagtggcagtggttctgggacagatttcactctcaccatcagcagtctacaacctgaagattttgcaacttactactgtcaacagacttacgacacgcctcccactttcggccctgggaccaaagttgatatcaaacgaactgtggctgcacc

SEQ ID NO: 228 - protein, 106 aa, Homo sapiens
SPSSLSASVGDTVTITCRASQNIYYYLYWYQQKPGKAPNLLIYGASSLQSGVPSRFSGSGSGTDFTLTISSLQPEDFATYYCQQTYDTPPTFGPGTKVDIKRTVAA

SEQ ID NO: 229 - DNA, 320 bp, Homo sapiens
tctccttccaccctgtctgcatttgtaggagacagagtcaccatcacttgccgggccagtcaaagtattagtacctggttggcctggtatcagcaaaaaccagggaaagcccctaacctcctgatctataaggcgtctaatttagaaagtggggtcccatcaaggttcagcggcagtggatctgggacagagttcactctcaccatcagcagcctgcagcctgatgattttgcaacttattactgccagcagtttagaagtcattcgtacacttttggccaggggaccaagttagagatcaaacgaactgtggctgcacc

SEQ ID NO: 230 - protein, 106 aa, Homo sapiens
SPSTLSAFVGDRVTITCRASQSISTWLAWYQQKPGKAPNLLIYKASNLESGVPSRFSGSGSGTEFTLTISSLQPDDFATYYCQQFRSHSYTFGQGTKLEIKRTVAA

SEQ ID NO: 231 - DNA, 308 bp, Homo sapiens
tctccctccaccctgtctgcatctgtaggagacagagtcaccatcacttgccgggccagtcagagtagtagtaactggttggcctggtatcagcagaaacctgggaaaggccccaaactcctgatctataaggcgtctaatttagaaagtggggtcccatcaaggttcagcggcagtggatctgggacagaattcactctcaccatcagcagcctgcagcctgatgattttgcaacttactactgccaacagtccgtgacgttcggccaagggaccaaggtggaaatcaaacgaactgtggctgcacc

SEQ ID NO: 232 - protein, 102 aa, Homo sapiens
SPSTLSASVGDRVTITCRASQSSSNWLAWYQQKPGKGPKLLIYKASNLESGVPSRFSGSGSGTEFTLTISSLQPDDFATYYCQQSVTFGQGTKVEIKRTVAA

SEQ ID NO: 233 - DNA, 308 bp, Homo sapiens
tctccctccaccctgtctgcatctgtaggagacagagtcaccatcacttgccgggccagtcagagtagtagtaactggttggcctggtatcagcagaaacctgggaaaggccccaaactcctgatctataaggcgtctaatttagaaagtggggtcccatcaaggttcagcggcagtggatctgggacagaattcactctcaccatcagcagcctgcagcctgatgattttgcaacttactactgccaacagtccgtgacgttcggccaagggaccaaggtggaatcaggacgaactgtggctgcacc

SEQ ID NO: 234 - protein, 102 aa, Homo sapiens
SPSTLSASVGDRVTITCRASQSSSNWLAWYQQKPGKGPKLLIYKASNLESGVPSRFSGSGSGTEFTLTISSLQPDDFATYYCQQSVTFGQGTKVESGRTVAA

SEQ ID NO: 235 - DNA, 320 bp, Homo sapiens
tctccatcctcactgtctgcatctgtaggagacagagtcaccatcacttgtcgggcgagtcagggtattggcagctggttagcctggtatcagcagaaaccagagaaagcccctaagtccctgatctatgctgcgtccactttgcaaagtggggtcccatcaaggttcagcggcggtgggtctgggacagatttcactctcaccatcaccagcctgcagcctgaagattttgcaacttattactgccaacagtataatagttacccgtacacttttggccaggggaccaagctggagatcaaacgaactgtggctgcacc

SEQ ID NO: 236 - protein, 106 aa, Homo sapiens
SPSSLSASVGDRVTITCRASQGIGSWLAWYQQKPEKAPKSLIYAASTLQSGVPSRFSGGGSGTDFTLTITSLQPEDFATYYCQQYNSYPYTFGQGTKLEIKRTVAA

SEQ ID NO: 237 - DNA, 320 bp, Homo sapiens
tctccacccaccctgtctttgtctccaggggacagagccaccctctcttgcagggccagtgagagtattggcaggcgcttagcctggtaccaacagaaacctggccaggctcccaggctcctcatccatgatgcatctcacagggccagtggcatcccacccaggttcagtggcagtgggtctggcacagacttcactctcaccatcagcagcctagagcctgacgattttgcaatttattactgtcaacagctcacaacctggtcgtacacttttggccaggggaccaaggtggagatcagacgaactgtggctgcacc

SEQ ID NO: 238 - protein, 106 aa, Homo sapiens
SPPTLSLSPGDRATLSCRASESIGRRLAWYQQKPGQAPRLLIHDASHRASGIPPRFSGSGSGTDFTLTISSLEPDDFAIYYCQQLTTWSYTFGQGTKVEIRRTVAA

SEQ ID NO: 239 - DNA, 326 bp, Homo sapiens
tctccaggcaccctgtctttgtctccaggggaaagagccaccctctcctgcagggccagtcagaatattatcagcagttacataacttggtaccagcacaaacctggccagcctcccaggctcctcatctatggtgcatctagcagggccactggcatcccagacagattcagtggcagtgggtctgggacagacttcactctcaccatcagcagactggagcctgaagattttgcagtatattactgtcaacactatggtagttcacttccgtacccctttgggcaggggaccaagctggagatcaaaggaactgtggctgcacc

SEQ ID NO: 240 - protein, 108 aa, Homo sapiens
SPGTLSLSPGERATLSCRASQNIISSYITWYQHKPGQPPRLLIYGASSRATGIPDRFSGSGSGTDFTLTISRLEPEDFAVYYCQHYGSSLPYPFGQGTKLEIKGTVAA

SEQ ID NO: 241 - DNA, 323 bp, Homo sapiens
tctccaggcaccctgtctttgtctccaggggaaagagccaccctctcctgcagggccagtcagagtgttagcagcacctacttagcctggtaccagcagaaacctggccaggctcccaggctcctcatctatggtgcatccaacagggccactggcatcccagacaggttcagtggcagtgggtctgggacagacttcactctcaccatcagcagactggagcctgaagattttgcagtgtattactgtcagcaatatggtagctcaccggccactttcggcggagggaccaaggtggagatcaaacgaactgtggctgcacc

SEQ ID NO: 242 - protein, 107 aa, Homo sapiens
SPGTLSLSPGERATLSCRASQSVSSTYLAWYQQKPGQAPRLLIYGASNRATGIPDRFSGSGSGTDFTLTISRLEPEDFAVYYCQQYGSSPATFGGGTKVEIKRTVAA

SEQ ID NO: 243 - DNA, 323 bp, Homo sapiens
tctccaggcaccctatctttgtctccaggggaaagaaccaccctctcctgcagggccagtcagagtgttaggggcacctacatagcctggtaccagcagagacctggccaggctcccaggctcctcatctatggtgcatccagcagggccactggcatcccagacaggttcagtggcagtgggtctgggacagacttcactctcaccgtcagcagactggagcctgaagattttgcagtgtattactgtcagcagtatggtagctcaccgtggacgttcggccaagggaccaaggtggaaatcaaacgaactgtggctgcacc

SEQ ID NO: 244 - protein, 107 aa, Homo sapiens
SPGTLSLSPGERTTLSCRASQSVRGTYIAWYQQRPGQAPRLLIYGASSRATGIPDRFSGSGSGTDFTLTVSRLEPEDFAVYYCQQYGSSPWTFGQGTKVEIKRTVAA

SEQ ID NO: 245 - DNA, 338 bp, Homo sapiens
tctccaaactccctggctgtgtctctgggcgagagggccaccatcaactgcaagtccagccagagtgttttatacagctccaacaataagaacttcttagcttggtaccagcagagaccaggacagcctcctaagctgctcttttactgggcatctacccgggaatccggggtccctgaccgattcagtggcagcgggtctgggacagatttcactctcaccatcagcagcctgcaggctgaagatgtggcaatttattactgtcagcagtatcacagtactcctccgacgttcggccaagggaccaaggtggagatcaaacgaactgtggctgcacc

SEQ ID NO: 246 - protein, 112 aa, Homo sapiens
SPNSLAVSLGERATINCKSSQSVLYSSNNKNFLAWYQQRPGQPPKLLFYWASTRESGVPDRFSGSGSGTDFTLTISSLQAEDVAIYYCQQYHSTPPTFGQGTKVEIKRTVAA

SEQ ID NO: 247 - DNA, 338 bp, Homo sapiens
tctccaaactccctggctgtgtctctgggcgagagggccaccatcaactgcaagtccagccagagtgttttatacagctccaacaataagaacttcttagcttggtaccagcagagaccaggacagcctcctaagctgctcttttactgggcatctacccgggaatccggggtccctgaccgattcagtggcagcgggtctgggacagatttcactctcaccatcagcagcctgcaggctgaagatgtggcaatttattactgtcagcagtatctcagtactcctccgacgttcggccaagggaccaaggtggagatcaaacgaactgtggctgcacc

SEQ ID NO: 248 - protein, 112 aa, Homo sapiens
SPNSLAVSLGERATINCKSSQSVLYSSNNKNFLAWYQQRPGQPPKLLFYWASTRESGVPDRFSGSGSGTDFTLTISSLQAEDVAIYYCQQYLSTPPTFGQGTKVEIKRTVAA

SEQ ID NO: 249 - DNA, 338 bp, Homo sapiens
tctccagactccctggctgtgtctctgggcgagagggccaccatcaactgcaagtccagccagagtgttttacacagctccaacaataagaactacttagcttggtaccagcagaaaccaggacagcctcctaagctgctcatttactgggcatctatccgggaatccggggtccctgagcgattcagtggcagcgggtctgggacagatttcactctcaccatcagcagcctgcaggctgaagatgtggcagtttattactgtcagcaatattatagttctcctctcactttcggcggagggaccaaggtggagatcaaacgaactgtggctgcacc

SEQ ID NO: 250 - protein, 112 aa, Homo sapiens
SPDSLAVSLGERATINCKSSQSVLHSSNNKNYLAWYQQKPGQPPKLLIYWASIRESGVPERFSGSGSGTDFTLTISSLQAEDVAVYYCQQYYSSPLTFGGGTKVEIKRTVAA

SEQ ID NO: 251 - DNA, 335 bp, Homo sapiens
tctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgacgttcggccaagggaccaaggtggaaatcaaacgaactgtggctgcacc

SEQ ID NO: 252 - protein, 111 aa, Homo sapiens
SPDSLAVSLGERATINCKSSQSVLYSSNNKNYLAWYQQKPGQPPKLLIYWASTRESGVPDRFSGSGSGTDFTLTISSLQAEDVAVYYCQQYYSTPTFGQGTKVEIKRTVAA

SEQ ID NO: 253 - DNA, 445 bp, Homo sapiens
ctcgagtctggggccgaggtgaagaagcctggggcctcagtgaaggtttcgtgcacgacatctggatacaccttcggcgaccactatatgcactgggtgcggcaggcccctggacaaaggcctgagtggttgggaataatcaaccctaggagcggtaggacaacctacgcacagaagttccagggcagagtcaccatgaccagcgacacgtccacgagcacgttctacatggagctgagcggcctgagatttgatgacacggccatgtatttctgtggaagagatgttagacgggcgcctcggtcagtcatcacaccccaagattggttcgacccctggggccagggaaccctggtcaccgtctccttggcctccaccaagggcccatcggtcttccccctggcaccctcctccaagagcacctctgggggcacagcggccctgg

SEQ ID NO: 254 - protein, 148 aa, Homo sapiens
LESGAEVKKPGASVKVSCTTSGYTFGDHYMHWVRQAPGQRPEWLGIINPRSGRTTYAQKFQGRVTMTSDTSTSTFYMELSGLRFDDTAMYFCGRDVRRAPRSVITPQDWFDPWGQGTLVTVSLASTKGPSVFPLAPSSKSTSGGTAAL

SEQ ID NO: 255 - DNA, 445 bp, Homo sapiens
ctcgagtctgggggaggcttggtcaagcctggagggtccctgagactctcctgtgcagcctctggattcagcttcagtgactactacatgacctggatccgccaggctccagggaaggggctggagtggctttcatacattagtggtagtggtcgcaccatatactacgcagactctgtgaagggccgattcaccatctccagggacgacgccaagaactccctgtatctgcaaatgaacagcctgagagccgaggacacggccgtgtattactgtgcgagagattccccaacgaggacgtattccgattttaggggtggtcccaaccagcccgaatactactactacggtatggacgtctggggccaagggaccacggtcaccgtctcctcagcctccaccaagggcccatcggtcttccccctggcaccctcctccaagagca

SEQ ID NO: 256 - protein, 148 aa, Homo sapiens
LESGGGLVKPGGSLRLSCAASGFSFSDYYMTWIRQAPGKGLEWLSYISGSGRTIYYADSVKGRFTISRDDAKNSLYLQMNSLRAEDTAVYYCARDSPTRTYSDFRGGPNQPEYYYYGMDVWGQGTTVTVSSASTKGPSVFPLAPSSKS

SEQ ID NO: 257 - DNA, 445 bp, Homo sapiens
ctcgagtctgggggaggcttggtgaagcctggagggtccctgagactctcctgtggagcctctggattcaggttcagtgactaccacatgagttggatccgccaggctccagggaagggcctggagtgggtctcacacattagtggtagtggcgtttccaaatactacgcagactctgtgaagggccgaatcaccatctccagggacaacgccaagaattcactgtatctacaaatggacagcctgagagacgaggacacggccgtatattactgtgcgagagagtcgtggctggcaatagaccactggggccagggaaccctggtcaccgtctcctcagcctccaccaagggcccatcggtcttccccctggcaccctcctccaagagcacctctgggggcacagcggccctgggctgcctggtcaaggactacttccccgaac

SEQ ID NO: 258 - protein, 148 aa, Homo sapiens
LESGGGLVKPGGSLRLSCGASGFRFSDYHMSWIRQAPGKGLEWVSHISGSGVSKYYADSVKGRITISRDNAKNSLYLQMDSLRDEDTAVYYCARESWLAIDHWGQGTLVTVSSASTKGPSVFPLAPSSKSTSGGTAALGCLVKDYFPE

SEQ ID NO: 259 - DNA, 445 bp, Homo sapiens
ctcgagtcggggggaaacttggcacagccgggggggtccctgagagtctcctgtgcagcctccggattcatgttcgggaattatgacatgttttgggtccgccaggctccagggaaggggctggagtgggtctcagggatcgatggtcgcagtgagaagacatactacgcagactccgtgaagggccggttcagcgtctccagagacaattccaagaacacactgtatttacaattgaacagactgagagccgaagacacggccgtttattactgtgcgaaacccccggacagtgggagccccatctattttgacaactggggccagggaaccctggtcaccgtctcgtcagcctccaccaagggcccatcggtcttccccctggcaccctcctccaagagcacctctgggggcacagcggccctgggctgcctggtcaaggact

SEQ ID NO: 260 - protein, 148 aa, Homo sapiens
LESGGNLAQPGGSLRVSCAASGFMFGNYDMFWVRQAPGKGLEWVSGIDGRSEKTYYADSVKGRFSVSRDNSKNTLYLQLNRLRAEDTAVYYCAKPPDSGSPIYFDNWGQGTLVTVSSASTKGPSVFPLAPSSKSTSGGTAALGCLVKD

SEQ ID NO: 261 - DNA, 445 bp, Homo sapiens
ctcgagtcggggggaggcgtggtccggcccgggacgtccctgacactctcctgtgcagcctctggattcgtcttcacaacttatggcatgcactgggtccgccaggctccagggaaggggccggagtgggtggcagtcatttcaaccgatggaaataaaaaagcctatggcaactccgtgaagggccgattcaccatctccagagacagattcagcaacacggtgtctttgcaaatggacagcctgagaccagatgacacggctatttattactgcgcgaaggaagggctgcgtgggacttacgttcgaggtgacctccagcattggggccagggcaccctggtcgtcgtctcttcggcctccaccaagggcccatcggtcttccccctggcaccctcctccaagagcacctctgggggcacagcggccctgggctgcctggtca

SEQ ID NO: 262 - protein, 148 aa, Homo sapiens
LESGGGVVRPGTSLTLSCAASGFVFTTYGMHWVRQAPGKGPEWVAVISTDGNKKAYGNSVKGRFTISRDRFSNTVSLQMDSLRPDDTAIYYCAKEGLRGTYVRGDLQHWGQGTLVVVSSASTKGPSVFPLAPSSKSTSGGTAALGCLV

SEQ ID NO: 263 - DNA, 445 bp, Homo sapiens
ctcgaggagtctgggggaggcgtggtccagcctggggggtccctgagactctcctgtgcagcctctggattttccttcagtaactatggcatgcactgggtccgccaggctccaggcaaggggctggagtgggtaactcttatatcagatgatggaagtaataaattctatgcagactccgtgaagggccgattcaccatctccagagacaattccaaaaacacgttgtatgtgcaaatgaacagcctgagacctgaagacacggctatatactactgtgcgaaagggtcccgtgatctcagtggttactattcgccggactactggggccagggaaccctggtcaccgtctcctcagcctccaccaagggcccatcggtcttccccctggcaccctcctccaagagcacctctgggggcacagcggccctgggctgcctggtca

SEQ ID NO: 264 - protein, 148 aa, Homo sapiens
LEESGGGVVQPGGSLRLSCAASGFSFSNYGMHWVRQAPGKGLEWVTLISDDGSNKFYADSVKGRFTISRDNSKNTLYVQMNSLRPEDTAIYYCAKGSRDLSGYYSPDYWGQGTLVTVSSASTKGPSVFPLAPSSKSTSGGTAALGCLV

SEQ ID NO: 265 - DNA, 445 bp, Homo sapiens
ctcgaggagtctgggggaggcgtggtccagcctgggaggtccctgagactctcctgtgcagcctctggattccccttcagtagttatggcatgcactgggtccgccaggctccaggcaaggggctggagtgggtggcaggtgtttcatatgatggaagttataaatactatgcggactccgtcaagggccgattcaccatctccagagacagttccaagagcactctatatctgcaaatgaacagcctgagacctgaggacacggctgtgtattactgtgcgagaccttccgcgatttttggaatatacattattctaaacggtttggacgtctggggccaagggaccacggtcaccgyctcttcagcctccaccaagggcccatcggacttccccctggmaccctcctccaagagcacctctggrggcacagcggccctgggct

SEQ ID NO: 266 - protein, 148 aa, Homo sapiens
LEESGGGVVQPGRSLRLSCAASGFPFSSYGMHWVRQAPGKGLEWVAGVSYDGSYKYYADSVKGRFTISRDSSKSTLYLQMNSLRPEDTAVYYCARPSAIFGIYIILNGLDVWGQGTTVTVSSASTKGPSDFPLAPSSKSTSGGTAALG

SEQ ID NO: 267 - DNA, 445 bp, Homo sapiens
ctcgagtctgggggaggcttggtccagcctggggggtccctgagactctcctgtgcagcctctggattcagcgtcagtagcatctatatgagctgggtccgccaggctccagggaaggggctggagtgggtctcacttatttatgacggtggtagcacatactacgcagactccgtgaagggccgattcaccgtctccagggacaattccaagaacacgctctatcttcaaatgaacagcctgagaggtgaagacacggctatatattactgtgcgagaggggtcgaggactattacactgataccagtggtttttacctgggttttgcctactggggccagggaaccccggtcaccgtctcctcagcctccaccaagggcccatcggtcttccccctggcaccctcctccaagagcacctctgggggcacagcggccctgggct

SEQ ID NO: 268 - protein, 148 aa, Homo sapiens
LESGGGLVQPGGSLRLSCAASGFSVSSIYMSWVRQAPGKGLEWVSLIYDGGSTYYADSVKGRFTVSRDNSKNTLYLQMNSLRGEDTAIYYCARGVEDYYTDTSGFYLGFAYWGQGTPVTVSSASTKGPSVFPLAPSSKSTSGGTAALG

SEQ ID NO: 269 - DNA, 445 bp, Homo sapiens
ctcgagcagtctgggggaggcttggtccaaccgggggggtccctgagactctcctgtgcaggctctggattcagtttcaaaagttatttcatgagttgggtccgccaggctccagggaaggggctggagtgggtggccaacataaagcaatatggaggcgacaaatactatgcggactctgtgaaaggacgattcaccatctccagagacgacgccaagaatttagtgtatctggaaatgaagagcctgagagccgacgacacggccgtgtattactgtgcgagaggatccctagagggattttttgagttcggtcagttaagtccgggatggttcgacttctggggccagggaaccctggtcaccgtctcctcagcctccaccaagggcccatcggtcttccccctggcaccctcctccaagagcacctctgggggcacagcgg

SEQ ID NO: 270 - protein, 148 aa, Homo sapiens
LEQSGGGLVQPGGSLRLSCAGSGFSFKSYFMSWVRQAPGKGLEWVANIKQYGGDKYYADSVKGRFTISRDDAKNLVYLEMKSLRADDTAVYYCARGSLEGFFEFGQLSPGWFDFWGQGTLVTVSSASTKGPSVFPLAPSSKSTSGGTA

SEQ ID NO: 271 - DNA, 445 bp, Homo sapiens
ctcgagtcgggcccaggactggtgaagccttcacagaccctgtccctcacctgcgttgtctctggtgactccatggataggggtggatacgcttggagctggaaccggcagccaccagggaagggactggagtggattgggtatatctattacagagggaccacctactacagcccgtccctcaagagtcgagtcaccatgtctttagacacgtccaacaaccagatctccctgaaactgagctctgtgaccgccgcggacacggccgtctattattgtgccagagtaccactcctaaattacgatattttgactggttattatactgtgaatgcttttgatgtctggggccaagggacaatggtcaccgtctcttcagcctccaccaagggcccatcggtcttccccctggcaccctcctccaagagcacctctgggggcacag

SEQ ID NO: 272 - protein, 148 aa, Homo sapiens
LESGPGLVKPSQTLSLTCVVSGDSMDRGGYAWSWNRQPPGKGLEWIGYIYYRGTTYYSPSLKSRVTMSLDTSNNQISLKLSSVTAADTAVYYCARVPLLNYDILTGYYTVNAFDVWGQGTMVTVSSASTKGPSVFPLAPSSKSTSGGT

SEQ ID NO: 273 - DNA, 445 bp, Homo sapiens
ctcgagtcgggcccaggactggtgaagccttcgcagaccctgtccctcacctgcgctgtctctggtggctccatcagcagtgaaggtttctcctggagttggatccggcagccaccagggaagggactggagttcattggttatatttattacaatgggaggacctatttcaacccgtccctcaggagtcgagttagcatttccgcagacatgtccaagaaccaattttccctgaaactgccctctgtgaccgccgcggacacggccgtctatttctgtgccagtacaataggatacacctatggcccggaatacttccatcactggggccagggcacccgggtcaccgtctcctcagcctccaccaagggcccatcggtcttccccctggcaccctcctccaagagcacctctgggggcacagcggccctgggctgcctggtca

SEQ ID NO: 274 - protein, 148 aa, Homo sapiens
LESGPGLVKPSQTLSLTCAVSGGSISSEGFSWSWIRQPPGKGLEFIGYIYYNGRTYFNPSLRSRVSISADMSKNQFSLKLPSVTAADTAVYFCASTIGYTYGPEYFHHWGQGTRVTVSSASTKGPSVFPLAPSSKSTSGGTAALGCLV

SEQ ID NO: 275 - DNA, 445 bp, Homo sapiens
ctcgagtcgggcccaggactggtgaagccttcggagaccctgtccctcacctgcactgtctctggtggctccgtcaccagtagtagttatctctggggctggatccgccagcccccagggaaggggctggactggattgggagtagtcattatagtgggagcacctaccacaacccgtccctcaagagtcgagtcaccacatccgtagacacgtccaagaaccggttctccctgaagctgagctctgtgaccgccgcagacacggctgtatattactgtgcgagacatgttgagggtgactacggtgacttttttgaccactggggccagggaaccctggtcaccgtctcctcagcctccaccaagggcccatcggtcttccccctggcaccctcctccaagagcacctctgggggcacggcggccctgggctgcctggtcaagg

SEQ ID NO: 276 - protein, 148 aa, Homo sapiens
LESGPGLVKPSETLSLTCTVSGGSVTSSSYLWGWIRQPPGKGLDWIGSSHYSGSTYHNPSLKSRVTTSVDTSKNRFSLKLSSVTAADTAVYYCARHVEGDYGDFFDHWGQGTLVTVSSASTKGPSVFPLAPSSKSTSGGTAALGCLVK

SEQ ID NO: 277 - DNA, 445 bp, Homo sapiens
ctcgagtcgggcccaggactggtgaagccttcggagaccctgtccctcacctgcacagtctctgctggctccatcagtagcaacagttatcactggggctggatccggcagcccccaggaaaggggctggaatggattggccatatttattatagtgggtccaccgactacaatccgtcccttcagagtcgagtcaccatatccattgacacgtccatgaatcgcttctccctaagggtgaactctgtgaccgccgcagacacggctgtatatttctgtgcgagattctacggtagttcatatgactactggggccggggaaccctggtcgccgtctcctcagcctccaccaagggcccatcggtcttccccctggcaccctcctccaagagcacctctgggggcacagcggccctgggctgcctggtcaaggactacttccccg

SEQ ID NO: 278 - protein, 148 aa, Homo sapiens
LESGPGLVKPSETLSLTCTVSAGSISSNSYHWGWIRQPPGKGLEWIGHIYYSGSTDYNPSLQSRVTISIDTSMNRFSLRVNSVTAADTAVYFCARFYGSSYDYWGRGTLVAVSSASTKGPSVFPLAPSSKSTSGGTAALGCLVKDYFP

SEQ ID NO: 279 - DNA, 445 bp, Homo sapiens
ctcgagtcgggcccaggactggtgaagccttcggggaccctgtccctcacctgcgctgtttctggtggctccatcagcagtagttactggtggaattgggtccgccagccccccgggaaggggctggagtggattggggaaatctatcatagtggggtcaccaactccaacccgtccctcaagagtcgagtcaccatatcagtagacaagtcgaacaatcgctttaccctagagttgaactctgtgaccgccgcggacacggccgtctattactgtgcgcgagatggaggccggggatattgtagtggtaatagctgccactccgggtctctcccccccccctggttcgacccctggggccagggaatcctggtcaccgtctcctcagcctccaccaagggcccatcggtcttccccctggcaccctcctccaagagcacctctg

SEQ ID NO: 280 - protein, 148 aa, Homo sapiens
LESGPGLVKPSGTLSLTCAVSGGSISSSYWWNWVRQPPGKGLEWIGEIYHSGVTNSNPSLKSRVTISVDKSNNRFTLELNSVTAADTAVYYCARDGGRGYCSGNSCHSGSLPPPWFDPWGQGILVTVSSASTKGPSVFPLAPSSKSTS

SEQ ID NO: 281 - DNA, 444 bp, Homo sapiens
ctcgagtcgggcccaggactggtgaagccttcacagaccctgtccctcacctgcactgtctctggtgcctccatcagcagtgaaacttactactggagctggatccggcagcccgccgggaagggactggagtggattgggcgtatgtataccagcgggagtagcaactacaacccctccctcaagagtcgagtctccatgtcggtcgacacgtccaagaaccagttctccctgaacctgaattctgtgaccgccgcagacacggccgtgtattattgtgcgagagatgtattggtcactatgattcgggggaatgtttttgacatatggggccaagggacagtggtcaccgtctcttcagcctccaccaagggcccatcggtcttccccctggcaccctcctccaagagcacctctgggggcacagcggccctgggctgcctg

SEQ ID NO: 282 - protein, 148 aa, Homo sapiens
LESGPGLVKPSQTLSLTCTVSGASISSETYYWSWIRQPAGKGLEWIGRMYTSGSSNYNPSLKSRVSMSVDTSKNQFSLNLNSVTAADTAVYYCARDVLVTMIRGNVFDIWGQGTVVTVSSASTKGPSVFPLAPSSKSTSGGTAALGCL

SEQ ID NO: 283 - DNA, 444 bp, Homo sapiens
ctcgagtcgggcccaggactggtgaagccttctcagaccctgtccctcacctgcactgtctctggtgcctccatcagcagtgaaacttactactggagctggatccggcagcccgccgggaagggactggagtggattgggcgtatgtataccagcgggagtagcaactacaacccctccctcaagagtcgagtctccatgtcggtcgacacgtccaagaaccagttctccctgaacctgaattctgtgaccgccgcagacacggccgtgtattattgtgcgagagatgtattggtcactatgattcgggggaatgtttttgacatatggggccaagggacagtggtcaccgtctcttcagcctccaccaagggcccatcggtcttccccctggcaccctcctccaagagcacctctgggggcacagcggccctgggctgcctg

SEQ ID NO: 284 - protein, 148 aa, Homo sapiens; identical to SEQ ID NO: 282

SEQ ID NO: 285 - DNA, 445 bp, Homo sapiens
ctcgagtcgggcccaggacaggtgaagccttcacagaccctgtccctcacctgcactgtctctggtggctccatcggcagtggttcttactcctggaactggatccggcagcccgccgggaggggactggagtggattgggcgaatctctgacagtgggaacaccaatttcaacccctccctcaagagtcgagtcaccatgtcagtggacacgtccaagaaccagttcgccctgaaactgacctctgtgaccgccgcagacacggccacatatttctgtgcgagagggagaggtattttgactggtctctttgactattggggccagggatccctggtctccgtctcctcagcctccaccaagggcccatcggtcttccccctggcaccctcctccaagagcacctctgggggcacagcggccctgggctgcctggtcaaggact

SEQ ID NO: 286 - protein, 148 aa, Homo sapiens
LESGPGQVKPSQTLSLTCTVSGGSIGSGSYSWNWIRQPAGRGLEWIGRISDSGNTNFNPSLKSRVTMSVDTSKNQFALKLTSVTAADTATYFCARGRGILTGLFDYWGQGSLVSVSSASTKGPSVFPLAPSSKSTSGGTAALGCLVKD

SEQ ID NO: 287 - DNA, 445 bp, Homo sapiens; identical to SEQ ID NO: 285

SEQ ID NO: 288 - protein, 148 aa, Homo sapiens; identical to SEQ ID NO: 286

SEQ ID NO: 289 - DNA, 445 bp, Homo sapiens
ctcgagtcgggcccaggactggtgaagccttcagagaccctgtccctcacctgcactgtctctggtggctccatcagcagtgggagtgactactggagctggatccggcagcccgccgggaaggggctggagtggattgggcgaatctccaccaaagggagcaccagctacaacccctccctccagagtcgagtcatcatatcactagacacgtccaagaaccagttttccctgaagctgaggtctgtgaccgccgcagacacggccctttattactgagcgagagctttcccgccggagaaggcagcagctggcactttcgacccttggggtcagggaaccctggtcatcgtctcctcagcctccaccaagggcccatcggtcttccccctggcaccctcctccaagagcacctctgggggcacagcggccctgggctgcctgg

SEQ ID NO: 290 - protein, 147 aa, Homo sapiens
LESGPGLVKPSETLSLTCTVSGGSISSGSDYWSWIRQPAGKGLEWIGRISTKGSTSYNPSLQSRVIISLDTSKNQFSLKLRSVTAADTALYYARAFPPEKAAAGTFDPWGQGTLVIVSSASTKGPSVFPLAPSSKSTSGGTAALGCL

SEQ ID NO: 291 - DNA, 445 bp, Homo sapiens
ctcgagtcgggcccaggactggtgaagccttcagagaccctgtccctcacctgcactgtctctggtggctccatcagcagtgggagtgactactggagctggatccggcagcccgccgggaaggggctggagtggattgggcgaatctccaccaaagggagcaccagctacaacccctccctccagagtcgagtcatcatatcactagacacgtccaagaaccagttttccctgaagctgaggtctgtgaccgccgcagacacggccctttattactgtgcgagagctttcccgccggagaaggcagcagctggcactttcgacccttggggtcagggaaccctggtcatcgtctcctcagcctccaccaagggcccatcggtcttccccctggcaccctcctccaagagcacctctgggggcacagcggccctgggctgcctgg

SEQ ID NO: 292 - protein, 148 aa, Homo sapiens
LESGPGLVKPSETLSLTCTVSGGSISSGSDYWSWIRQPAGKGLEWIGRISTKGSTSYNPSLQSRVIISLDTSKNQFSLKLRSVTAADTALYYCARAFPPEKAAAGTFDPWGQGTLVIVSSASTKGPSVFPLAPSSKSTSGGTAALGCL

SEQ ID NO: 293 - DNA, 445 bp, Homo sapiens
ctcgagtcgggcccaggactggtgaagccttcacagaccctgtccctcacctgcactgtctctggtggctccatcagcagtggaagtgactactggtcctggatccggcagcccgccgggaagggactggagtggattggccgaatctccaccagagggagcaccagctacaacccctccctccagagtcgagtcaccatttcactagacacgtccaagaaccagttttccctgaagttgacctctgtgaccgccgcagacacggccctttatttttgtgcgagagctttcccgccggagaaaccagcagctggtactttcgacccctggggccagggaaccctggtcaccgtctcctcagcctccaccaagggcccatcggtcttccccctggcaccctcctccaagagcacctctgggggcacagcggccctgggctgcctgg

SEQ ID NO: 294 - protein, 148 aa, Homo sapiens
LESGPGLVKPSQTLSLTCTVSGGSISSGSDYWSWIRQPAGKGLEWIGRISTRGSTSYNPSLQSRVTISLDTSKNQFSLKLTSVTAADTALYFCARAFPPEKPAAGTFDPWGQGTLVTVSSASTKGPSVFPLAPSSKSTSGGTAALGCL

SEQ ID NO: 295 - DNA, 445 bp, Homo sapiens
ctcgagcagtctggggcggaggtgaagaagccgggggagtctctgaggatctcctgtaagggttctggatacagcttttccacctactggatcgcctgggtgcgccagatgcccgggaaaggcctggagtggatgggcatcatctatcctggtgactctgatgtcaagtacagcccgtctttccaaggccaggtcaccatctcagccgacaggtccatcggcgccgcctacctgcagtggagcagactgaaggcctcggacaccgccatgtatttctgtgcgagacaagatgataggggctattacttctatgactattggggccagggaaccctggtcaccgtctcctcagcttccaccaagggcccatcggtcttccccctggcgccctgctccaggagcacctctgggggcacagcggccctgggctgcctggtcaaggact

SEQ ID NO: 296 - protein, 148 aa, Homo sapiens
LEQSGAEVKKPGESLRISCKGSGYSFSTYWIAWVRQMPGKGLEWMGIIYPGDSDVKYSPSFQGQVTISADRSIGAAYLQWSRLKASDTAMYFCARQDDRGYYFYDYWGQGTLVTVSSASTKGPSVFPLAPCSRSTSGGTAALGCLVKD

SEQ ID NO: 297 - DNA, 324 bp, Homo sapiens
tctccatcttccgtgtctgcatctgtaggagacagagtcactatcacttgtcgggcgactcagggtattagtagttggttagcctggtatcaacagaaaccagggaaaccacctaaactcctgatttttggtgcatctagtttgcaaagtggggtcccatcaaggttcagcggcagtggatctgggacagatttcactctcaccatcagcagtctacagcctgaagattttgcaacttacttttgtcaacaggctcacagtttcccgctcactttcggcggcgggaccaaggtggagatcaaacgaactgtggctgcaccatct

SEQ ID NO: 298 - protein, 108 aa, Homo sapiens
SPSSVSASVGDRVTITCRATQGISSWLAWYQQKPGKPPKLLIFGASSLQSGVPSRFSGSGSGTDFTLTISSLQPEDFATYFCQQAHSFPLTFGGGTKVEIKRTVAAPS

SEQ ID NO: 299 - DNA, 324 bp, Homo sapiens
tctccatcctccctgtctgcatctgtaagagacagagtcaccatcacttgccaggcgagtcaggacattagcaccaatttaaattggtatcagaagaaatcaggcaaacctcctaagctcttgatctacgatgcatccaatttggaaacaggggtcccatcaaggtttggtggaagtggatctgggacagattttactttcaccatcagcaacctgcagcctgaagattttgcaacatattactgtcaacattatgataatgtcccattcactttcggccctgggaccaaagtggatatcagacgaactgtggctgcaccatct

SEQ ID NO: 300 - protein, 108 aa, Homo sapiens
SPSSLSASVRDRVTITCQASQDISTNLNWYQKKSGKPPKLLIYDASNLETGVPSRFGGSGSGTDFTFTISNLQPEDFATYYCQHYDNVPFTFGPGTKVDIRRTVAAPS

SEQ ID NO: 301 - DNA, 324 bp, Homo sapiens
tctccatcctccctgtctgcatctgtaggagacagagtcaccatcacttgccaggcgagtcaggacataagcagaaatttaaattggtatcagcaaaagccagggaaagcccctgtgctcctgatctacggtgcatccactttggaaacaggggtcccatcaaggttcagtggaggtggatctgggacagattttactttcaccatcagcagcctgcagcctgaagatgttgccacattctactgtcaacagtatgatgctctcccgtacacttttggcccggggaccaggctggagttcttacgaactgtggctgcaccatct

SEQ ID NO: 302 - protein, 108 aa, Homo sapiens
SPSSLSASVGDRVTITCQASQDISRNLNWYQQKPGKAPVLLIYGASTLETGVPSRFSGGGSGTDFTFTISSLQPEDVATFYCQQYDALPYTFGPGTRLEFLRTVAAPS

SEQ ID NO: 303 - DNA, 324 bp, Homo sapiens
tctccatcctccctgtctgcatctgtaggagacagagtcaccatcacttgccaggcgagtcaggacattagcacctatttaaattggtatcagcagaaaccagggaaagcccctaaactcctgatctacgatgcatccaatttggaaacacgggtcccatcaaggttcggtggaagtggatctggaaaagactttactctcaccatcaacagcctgcagcctgaagattttgcaacatattactgtcaacagtatgatcattacccgatcaccttcggccaagggacacgactggagattaaacgaactgtggctgcaccatct

SEQ ID NO: 304 - protein, 108 aa, Homo sapiens
SPSSLSASVGDRVTITCQASQDISTYLNWYQQKPGKAPKLLIYDASNLETRVPSRFGGSGSGKDFTLTINSLQPEDFATYYCQQYDHYPITFGQGTRLEIKRTVAAPS

SEQ ID NO: 305 - DNA, 324 bp, Homo sapiens
tctccatcctccctgtctgcatctgtaggagacagagtcaccatcacttgccaggcgagtcaggacattaacaacgacttaaattggtatcagcagaaaccagggaaagcccctaagctcctgatgtacgatgcatccaatttggaagtgggggtcccatttaggtacagtggaagtggatctgggacagattttactttcaccgtcggcagcctgcagcctgaagatgttgcaacatattactgtcaacagtatcatgatctccctcacacttttggccaggggaccaagctggagttctaacgaactgtggctgcaccatct

SEQ ID NO: 306 - protein, 108 aa, Homo sapiens
SPSSLSASVGDRVTITCQASQDINNDLNWYQQKPGKAPKLLMYDASNLEVGVPFRYSGSGSGTDFTFTVGSLQPEDVATYYCQQYHDLPHTFGQGTKLEFLRTVAAPS

SEQ ID NO: 307 - DNA, 324 bp, Homo sapiens
tctccactctccctgtctgcatctgtgggagacagaatcaccatctcttgccgggcaagtctgaccattggtagatatgtaaattggtatcagcagaggccaggggaagcccccaaactcctgatctatgctgcatctaccttgcatattgtggtcccatcaaggttcagtggcagtggctctggcacagatttcactctcaccatcaacagtctgcaacgtgaagactttgcaatttacttctgtcaagagaattacagtgccacgcgcacttttggccaggggaccaaggtggagatcaagcgaactgtggctgcaccatct

SEQ ID NO: 308 - protein, 108 aa, Homo sapiens
SPLSLSASVGDRITISCRASLTIGRYVNWYQQRPGEAPKLLIYAASTLHIVVPSRFSGSGSGTDFTLTINSLQREDFAIYFCQENYSATRTFGQGTKVEIKRTVAAPS

SEQ ID NO: 309 - DNA, 295 bp, Homo sapiens
tctccatcctccctgtctgcatctgtaggagacagagtcaccatcacttgccgggcaagtcagaacattggcatctatttaaattggtatcaccacaaaccagggcaagcccctgagctcctgatctttggtgcatccactttgcaaagtggggtcccatcaaggttcagtggcagtggatctgggacagatttcactctcaccatcagcagtstgcaacctgacgatttggcaacttactactgtcaacagagttacagtacccctctcaccttcggcggggggaccaaggtgg

SEQ ID NO: 310 - protein, 98 aa, Homo sapiens
SPSSLSASVGDRVTITCRASQNIGIYLNWYHHKPGQAPELLIFGASTLQSGVPSRFSGSGSGTDFTLTISSLQPDDLATYYCQQSYSTPLTFGGGTKV

SEQ ID NO: 311 - DNA, 295 bp, Homo sapiens
tctccatcctccctgtctgcttctgtaggagacagagtctccatcacttgccgggcaagtcagagcattagcaactatttaaattggtatcagcagacaccagggaaagcccctaaactcctgatctatgctgcatccagtttgcaaagtggggtcccatcaaggttcagtggcagtggatctgggacagatttcactctcaccatcagcagtctacaacctgaagattttgcaacttacttctgtcaacagagttacagtaccccgtggacgttcggccaagggaccaaggtgg

SEQ ID NO: 312 - protein, 98 aa, Homo sapiens
SPSSLSASVGDRVSITCRASQSISNYLNWYQQTPGKAPKLLIYAASSLQSGVPSRFSGSGSGTDFTLTISSLQPEDFATYFCQQSYSTPWTFGQGTKV

SEQ ID NO: 313 - DNA, 296 bp, Homo sapiens
tctccatcgtccctgtctgcatctataggagacatagtcaccatcacttgccgggcaagtcagggcacttccaattttgtaaattggtatcagcagaaaccagggaaagcccctaaactcctgatctatactgcatccactttgcaaagtggggtcccatcaaggttcagtggcagtggatctgggacagatttcactctcaccatcagcagtctacaacctgaagattttgcaacttacttctgtcaacagagttacagtaccccgtggacgttcggccaagggaccaaggtgga

SEQ ID NO: 314 - protein, 98 aa, Homo sapiens
SPSSLSASIGDIVTITCRASQGTSNFVNWYQQKPGKAPKLLIYTASTLQSGVPSRFSGSGSGTDFTLTISSLQPEDFATYFCQQSYSTPWTFGQGTKV

SEQ ID NO: 315 - DNA, 324 bp, Homo sapiens
tctccatcctcactgtctgcctctgtaggagacagagtcaccttgacttgccgggcaagtcagaccgttaacaactatttacattggtatcagcagaaaccagggaaagcccctaaactgctgatctacgcctcatccactttgcaaagtggggtcacgtcaaggttcagtggcagtggatctgggacagacttcactctcaccatcaccagtctcgaggttgacgattttgcaatttactactgtcaacagagttacagtaccccgtggacattcggccaagggaccaaagtggaaatcaaacgaactgtggctgcaccatct

SEQ ID NO: 316 - protein, 108 aa, Homo sapiens
SPSSLSASVGDRVTLTCRASQTVNNYLHWYQQKPGKAPKLLIYASSTLQSGVTSRFSGSGSGTDFTLTITSLEVDDFAIYYCQQSYSTPWTFGQGTKVEIKRTVAAPS

SEQ ID NO: 317 - DNA, 323 bp, Homo sapiens
tctccatccttcctgtctgcatctgtcggagacagagtcaccatcacttgccgggccagtcagggcattaccacttatttagcctggtatcagcaaaaaccagggagagcccctaagctcctgatctattctgcatccactttgcaaagaggggtcccatcaagattcagcggcagtggatctgggacagagttcgctctcacaatcagcagcctgcagcctgaagattctgcaacttattactgtcaagaacttgatagttacccctacacttttggccaggggaccaagctggagttccactaactgtggctgcaccatct

SEQ ID NO: 318 - protein, 101 aa, Homo sapiens
SPSFLSASVGDRVTITCRASQGITTYLAWYQQKPGRAPKLLIYSASTLQRGVPSRFSGSGSGTEFALTISSLQPEDSATYYCQELDSYPYTFGQGTKLEFH

SEQ ID NO: 319 - DNA, 345 bp, Homo sapiens
tctccactcaccctgcccgtcacccctggagagtcggcctccatctcctgcaaatctagtcagagcctcctgcagagtaatggatacaactatttggattggtacgtggtgaagccagggcagtctccacaactcctgatctacttgggctctaatcgggcctccggggtccctgacaggttcagtggcagtggatcaggcaccgattttacactggaaatcagtagagtggaggctgaggatgttggactttatttctgcatgcaagctctgcacactcctagcatgtacacttttggccaggggaccacggtggagatcaaacgaactgtggctgcaccatct

SEQ ID NO: 320 - protein, 115 aa, Homo sapiens
SPLTLPVTPGESASISCKSSQSLLQSNGYNYLDWYVVKPGQSPQLLIYLGSNRASGVPDRFSGSGSGTDFTLEISRVEAEDVGLYFCMQALHTPSMYTFGQGTTVEIKRTVAAPS

SEQ ID NO: 321 - DNA, 313 bp, Homo sapiens
tctccactcaccctgcccgtcacccctggagagtcggcctccatctcctgcaaatctagtcagagcctcctgcagagtaatggatacaactatttggattggtacgtggtgaagccagggcagtctccacaactcctgatctacttgggctctaatcgggcctccggggtccctgacaggttcagtggcagtggatcaggcaccgattttacactggaaatcagtagagtggaggctgaggatgttggactttatttctgcatgcaacatgcaagctctcgtagcatgtacacttttggccaggggaccacgg

SEQ ID NO: 322 - protein, 104 aa, Homo sapiens
SPLTLPVTPGESASISCKSSQSLLQSNGYNYLDWYVVKPGQSPQLLIYLGSNRASGVPDRFSGSGSGTDFTLEISRVEAEDVGLYFCMQHASSRSMYTFGQGTT

SEQ ID NO: 323 - DNA, 330 bp, Homo sapiens
tctccaggcaccctgtctttgtctccaggggaaagagccaccctctcctgcagggccagtcagagtgttagcagttcctctttggcctggtaccaacagaaacctggccaggctcccagggtcctcatctttgctgcagccagcagggccactggcatcccagacaggttcagtggcagtgggtctgggacagagttcactctcaccatcagcagggtggagcctgaagattttgcagtgtatttctgtcagcactatgataactcaccgaggttcacttttggccaggggaccaagctggagaactaacgaactgtggctgcaccatct

SEQ ID NO: 324 - protein, 102 aa, Homo sapiens
SPGTLSLSPGERATLSCRASQSVSSSSLAWYQQKPGQAPRVLIFAAASRATGIPDRFSGSGSGTEFTLTISRVEPEDFAVYFCQHYDNSPRFTFGQGTKLEN

SEQ ID NO: 325 - DNA, 330 bp, Homo sapiens
tctccaggcaccctgtctttgtctccaggagaaagagccaccctctcctgcagggccagtcagaatgttggcggcagctacttagcctggtaccagcagaaacctggccaggctcccaggctcctcatctatggtgcatccagcagggccactggcgtcccagacaggttcagtggcagtgggtctgggacagacttcattctcaccatcagcagactggagcctgaagattttgcagtgtattactgtcagcagtatggtagctcaccgggattcactttcggccctgggaccaaagtggatatcaaacgaactgtggctgcaccatct

SEQ ID NO: 326 - protein, 110 aa, Homo sapiens
SPGTLSLSPGERATLSCRASQNVGGSYLAWYQQKPGQAPRLLIYGASSRATGVPDRFSGSGSGTDFILTISRLEPEDFAVYYCQQYGSSPGFTFGPGTKVDIKRTVAAPS

SEQ ID NO: 327 - DNA, 330 bp, Homo sapiens
tctccaggcaccctgtctttgtctccaggggaaagagccaccctctcgtgcagggccagtcagagtgttagcaacaacttcttagcctggtaccagcagaaacctggccaggctcccaggctcctcatctatggtgcatccagcagggccactggcgtcccagacaggttcagtggcagtgggtctgggacagacttcattctcaccatcagcagactggagcctgaagattttgcagtgtattactgtcagcagtatggtagctcaccgggattcactttcggccctgggaccaaagtggatatcaaacgaactgtggctgcaccatct

SEQ ID NO: 328 - protein, 110 aa, Homo sapiens
SPGTLSLSPGERATLSCRASQSVSNNFLAWYQQKPGQAPRLLIYGASSRATGVPDRFSGSGSGTDFILTISRLEPEDFAVYYCQQYGSSPGFTFGPGTKVDIKRTVAAPS

SEQ ID NO: 329 - DNA, 330 bp, Homo sapiens
tctccaggcaccctgtctttgtctccaggggaaagagccaccctctcgtgcagggccagtcagagtgttagcaacaacttcttagcctggtaccagcagaaacctggccaggctcccaggctcctcatctatggtgcatccagcagggccactgacatcccagacaggttcagtggcagtgggtctgggacagacttcactctcaccatcagcagactggaggctgacgattttgctgtttattactgtcaacagtatgatacctcagttccggtcactttcggcggagggaccaaggtggaggtcttacgaactgtggctgcaccatct

SEQ ID NO: 330 - protein, 110 aa, Homo sapiens
SPGTLSLSPGERATLSCRASQSVSNNFLAWYQQKPGQAPRLLIYGASSRATDIPDRFSGSGSGTDFTLTISRLEADDFAVYYCQQYDTSVPVTFGGGTKVEVLRTVAAPS

SEQ ID NO: 331 - DNA, 330 bp, Homo sapiens
tctccaggcaccctgtctttgtctccaggggaaagagccaccctctcgtgcagggccagtcagagtgtcagcaacaacttcctagcctggtaccagcagaaacctggccaggctcccaggctcctcatctatggtgcatccagcagggccactgacatcccagacaggttcagtggcagtgggtctgggacagacttcactctcaccatcagcagactggaggctgaggattttgctgtttatcactgtcaacagtatggtacctcagttccggtcactttcggcggagggaccaaggtggagatcaaacgaactgtggctgcaccatct

SEQ ID NO: 332 - protein, 110 aa, Homo sapiens
SPGTLSLSPGERATLSCRASQSVSNNFLAWYQQKPGQAPRLLIYGASSRATDIPDRFSGSGSGTDFTLTISRLEAEDFAVYHCQQYGTSVPVTFGGGTKVEIKRTVAAPS

SEQ ID NO: 333 - DNA, 330 bp, Homo sapiens
tctccaggcaccctgtctttgtctccaggggaaagagccaccctctcgtgcagggccagtcagagtgttagcaacaacttcttagcctggtaccagcagaaacctggccaggctcccaggctcctcatctatggtgcatccagcagggccactgacatcccagacaggttcagtggcagtgggtctgggacagacttcactctcaccatcagcagactggaggctgaggattttgctgtttatcactgtcaacagtatggtacctcagttccggtcactttcggcggagggaccaaggtggagatcaaacgaactgtggctgcaccatct

SEQ ID NO: 334 - protein, 110 aa, Homo sapiens; identical to SEQ ID NO: 332

SEQ ID NO: 335 - DNA, 327 bp, Homo sapiens
tctccaggcaccctgtctttgtctccaggggaaagagccaccctctcctgcagggccagtcagagtgttagtaggagctacttagcctggtaccaacagaaacctggccaggctcccaggctcctcatctatggtgcatccagcagggccactggcatcccagacaggttcagtggcagtgggtctgggacagacttcactctcagcatcagcggactggagcctgaagattttgcagtgtatttctgtcagcagtttggtggctcacagtacacttttggccaggggaccaagctggagatcaaacgaactgtggctgcaccatct

SEQ ID NO: 336 - protein, 109 aa, Homo sapiens
SPGTLSLSPGERATLSCRASQSVSRSYLAWYQQKPGQAPRLLIYGASSRATGIPDRFSGSGSGTDFTLSISGLEPEDFAVYFCQQFGGSQYTFGQGTKLEIKRTVAAPS

SEQ ID NO: 337 - DNA, 316 bp, Homo sapiens
tctccagactccctggctgtgtctccgggcgggagggccaccatcaagtgcgcgtccagccagagtgttttggacaactccaaccataagaactccttggcgtggtaccagcagaaaccaggactgcctcctaaactgctcatttactgggcatctacccggtattccggggtccctgaccgattcagtggcagtgggtctgggacagatttcactctcaccatcaacaacctgcaggctgccgatgtggcagtttatttctgtcagcaatattatagtactccgtggaccttcggccaggggaccaaggtggagc

SEQ ID NO: 338 - protein, 105 aa, Homo sapiens
SPDSLAVSPGGRATIKCASSQSVLDNSNHKNSLAWYQQKPGLPPKLLIYWASTRYSGVPDRFSGSGSGTDFTLTINNLQAADVAVYFCQQYYSTPWTFGQGTKVE

SEQ ID NO: 339 - DNA, 342 bp, Homo sapiens
tctccagactccctggctgtgtctccgggcgggagggccaccatcaagtgcgcgtccagccagagtgttttggacaactccaaccataagaactccttggcgtggtaccagcagaaaccaggactgcctcctaaactgctcatttactgggcatctacccggtattccggggtccctgaccgattcagtggcagtgggtctgggacagatttcactctcaccatcagcagcctgcaggctgaggatgtggctttttattactgtcagcaatattatagtactccgtggaccttcggccaggggaccaaggtggagatcaaacgaactgtggctgcaccatct

SEQ ID NO: 340 - protein, 114 aa, Homo sapiens
SPDSLAVSPGGRATIKCASSQSVLDNSNHKNSLAWYQQKPGLPPKLLIYWASTRYSGVPDRFSGSGSGTDFTLTISSLQAEDVAFYYCQQYYSTPWTFGQGTKVEIKRTVAAPS

SEQ ID NO: 341 - DNA, 342 bp, Homo sapiens
tctccagactccctggctgtgtctctgggcgagagggccaccatcaactgcaagtccagccagagtcttttgtacagctccagcaataagaactacttagcttggtaccagcagaaaccaggacagtctcctaagttgctcatttactgggcttcttcccgggaatccggggtccctgcccgattcagtggcagcgggtctgggacagatttcactctcaccatcagcagcctgcaggctgaggatgtggctttttattactgtcagcaatattataatactcctcgaacgttcggccaggggaccaaggtggaagtcaaacgaactgtggctgcaccatct

SEQ ID NO: 342 - protein, 114 aa, Homo sapiens
SPDSLAVSLGERATINCKSSQSLLYSSSNKNYLAWYQQKPGQSPKLLIYWASSRESGVPARFSGSGSGTDFTLTISSLQAEDVAFYYCQQYYNTPRTFGQGTKVEVKRTVAAPS

SEQ ID NO: 343 - DNA, 310 bp, Homo sapiens
tctccagaatacttggctgtgtctctgggcgagagggccaccatcaactgcaagtccagccggagtgttttagacagctccaacaataagaacttcttggcctggtaccaacaaaaaccagggcagcctcctaaactactcatttattgggcatctacccgggaatccggggtccctgaccgattcagtggcagcgggtctgggacagatttcactctcaccattagcagcctgcaggctgttgatgtggcagtttattactgtcaggagtatttttgtactccgtggacgttcggccaagggaccaagg

SEQ ID NO: 344 - protein, 103 aa, Homo sapiens
SPEYLAVSLGERATINCKSSRSVLDSSNNKNFLAWYQQKPGQPPKLLIYWASTRESGVPDRFSGSGSGTDFTLTISSLQAVDVAVYYCQEYFCTPWTFGQGTK

SEQ ID NO: 345 - DNA, 350 bp, Homo sapiens
caggtgaaactgctcgagtcgggggctgaggtgaagaagcctggggcctcagtgaaggtctcctgcgagacttctggttacgattttaccagctacagtgtcaactgggtgcgacaggcccctggacaaggacttgagtggatgggatggatcagcccttacaatggtaagagaaactatgcacagactctccaggacagagtcaccttgaccaccgacacatccacgaacacagcctacatggaactgcggagcctgagatccgacgacacggccatttatttctgcgcgcgggaaggcagcagctggtacgagttggaccactggggccagggaatcctggtcaccgt

SEQ ID NO: 346 - protein, 116 aa, Homo sapiens
QVKLLESGAEVKKPGASVKVSCETSGYDFTSYSVNWVRQAPGQGLEWMGWISPYNGKRNYAQTLQDRVTLTTDTSTNTAYMELRSLRSDDTAIYFCAREGSSWYELDHWGQGILVT

SEQ ID NO: 347 - DNA, 350 bp, Homo sapiens
caggtgaaactgctcgagtcgggggctgaggtgaagaagcctggggcctcagtgaaggtctcctgcgagacttctggttacgattttaccagctacagtgtcaactgggtgcgacaggcccctggacaagggcttgagtggatgggatggatcagcccttacaatggtaagagaaactatgcacagactctccaggacagagtcaccttgaccaccgacacatccacgaacacagcctacatggaactgcggagcctgagatccgacgacacggccatttatttctgcgcgcgggaaggcagcagctggtacgagttggaccactggggccagggaatcctggtcaccgt

SEQ ID NO: 348 - protein, 116 aa, Homo sapiens; identical to SEQ ID NO: 346

SEQ ID NO: 349 - DNA, 350 bp, Homo sapiens
caggtgaaactgctcgagtctgggggaggcttggtcaagcctggagggtccctgagactctcctgtgcaggctccggattcaccttcagtgactattccatgagctgggtccgccacgctccagggaggggcctggagtggcttgcagacattactggtgttggtccttccgtgtactacgcagactctgtgaggggccgattcaccctctcccgggacaacgccaagaggtcactgtatctgcaaatggacagcctgagagtcgacgacacgggcaaatattactgtgccttgctctatggttcgcgaatgagcccctttgaccactggggccagggaacagtggtcac

SEQ ID NO: 350 - protein, 116 aa, Homo sapiens
QVKLLESGGGLVKPGGSLRLSCAGSGFTFSDYSMSWVRHAPGRGLEWLADITGVGPSVYYADSVRGRFTLSRDNAKRSLYLQMDSLRVDDTGKYYCALLYGSRMSPFDHWGQGTVV

SEQ ID NO: 351 - DNA, 303 bp, Homo sapiens
ggggaaagagccaccctctcctgcagggccagtcagactgttagtagcacctacttagcctggtaccagctaaaacctggccaggctcccaggctcctcatccatggtgcatccagcagggccactggcatcccagacaggttcagtggcggtgggtctgggacagacttcactctcaccatcagtagactggagcctgaagattttgcactgtattgctgtcaacactacggtagctcaccggggatcactttcggcggagggaccacggtggagatcaaacgaactgtggctgcaccatct

SEQ ID NO: 352 - protein, 101 aa, Homo sapiens
GERATLSCRASQTVSSTYLAWYQLKPGQAPRLLIHGASSRATGIPDRFSGGGSGTDFTLTISRLEPEDFALYCCQHYGSSPGITFGGGTTVEIKRTVAAPS

SEQ ID NO: 353 - DNA, 303 bp, Homo sapiens
ggagacagagtcaccatcacttgccgggcgagtcaggacattagcaattatttagcctggtttcagcagagacccgggaaagttcctaatctcctcatctatgctgcatccactttgcaatcaggggtcccatctcgattcagtggcagtggatctggaacagaattcactctcaccatcagtagtctgcagcctgaagatggtgcaagttattactgtcaaaagtatgacggtgccccttggacgttcggccaggggaccaaggtggaaatcaaacgaactgtggctgcaccatctgtcttc

SEQ ID NO: 354 - protein, 101 aa, Homo sapiens
GDRVTITCRASQDISNYLAWFQQRPGKVPNLLIYAASTLQSGVPSRFSGSGSGTEFTLTISSLQPEDGASYYCQKYDGAPWTFGQGTKVEIKRTVAAPSVF

SEQ ID NO: 355 - DNA, 303 bp, Homo sapiens
ggagacagagtcaccatcacttgccaggcgagtcaggacattatcacctatttaaattggtatcagcagaaaccagggaaagcccctaaactcctgatctacgaagcatccgatttggaaacaggggtcccatcaaggttcagtggaagtggatctgggacatattttactttcaccatcagtagcctgcagtctgaagatattgcaacatattactgtcaacaatttgatagtctccccctcactttcggcggagggaccaaggtggagatcaaacgaactgtggctgcaccatctgtctcc

SEQ ID NO: 356 - protein, 101 aa, Homo sapiens
GDRVTITCQASQDIITYLNWYQQKPGKAPKLLIYEASDLETGVPSRFSGSGSGTYFTFTISSLQSEDIATYYCQQFDSLPLTFGGGTKVEIKRTVAAPSVS

SEQ ID NO: 357 - DNA, 303 bp, Homo sapiens
ggggaaagagccaccctctcctgcagggccagtcagagtgttagcagcaacttagcctggtaccagcagaaacctggccaggctcccaggctcgtcatctatggtgcatccaccagggccactggtatcccagccaggttcagtggcagtgggtctgggacagacttcactctcaccatcgacagcctagagcctgaagattttgcagtttattactgtcagcagcgtagcgacttgcggtcgttcggccaggggaccaaggtggagatcaaacgaactgtggctgcaccatctgtcttcatc

SEQ ID NO: 358 - protein, 101 aa, Homo sapiens
GERATLSCRASQSVSSNLAWYQQKPGQAPRLVIYGASTRATGIPARFSGSGSGTDFTLTIDSLEPEDFAVYYCQQRSDLRSFGQGTKVEIKRTVAAPSVFI

SEQ ID NO: 359 - DNA, 303 bp, Homo sapiens
ggggaaagagccaccctctcctgcagggccagtcagagtgtcagcagtagttacttagcctggtatcaacagagacctggccggtctcccaggctcctcatctatggtgcatccagcagggccactggcatcccagacaggttcagtggcagtgggtctgggacagagttcactctcaccatcagcagcctgcagtctgaagattttgcagtttattactgtcagcagtatggcagcacaccgtacacttttggccaggggaccaaggtggagatgaaacgaactgtggctgcaccatctgtc

SEQ ID NO: 360 - protein, 101 aa, Homo sapiens
GERATLSCRASQSVSSSYLAWYQQRPGRSPRLLIYGASSRATGIPDRFSGSGSGTEFTLTISSLQSEDFAVYYCQQYGSTPYTFGQGTKVEMKRTVAAPSV

SEQ ID NO: 361 - DNA, 303 bp, Homo sapiens
ggggaaagagccaccctctcctgcagggccagtcagagtattaccagcagctacttagcctggttccagcagaaacctggccaggctcccaggctcctcatctttggtgcttccaccagggccacaggcatcccagacaggttcagtggcagtgggtctgggacagacttcactctctccatcagcagactggagcctgaagattttgcagtgtatttttgtcagcagtatggcagcacaccgtacacttttggccaggggaccaaggtggagatgaaacgaactgtggctgcaccatctgtc

SEQ ID NO: 362 - protein, 101 aa, Homo sapiens
GERATLSCRASQSITSSYLAWFQQKPGQAPRLLIFGASTRATGIPDRFSGSGSGTDFTLSISRLEPEDFAVYFCQQYGSTPYTFGQGTKVEMKRTVAAPSV

SEQ ID NO: 363 - DNA, 303 bp, Homo sapiens
ggggaaagagccaccctctcctgcagggccagtcagagtgtcagcacctatttagcctggtaccaacagaaagctggccagcctcccaggctcctcatccacgatgcttccaagagggccactggcatcccagccaggttcagtggcagtgggtctgggacagacttcactctcaccatcagcagcctggagcctgaggattttgcagtgtattactgtcagcagtatggtacctcaccgctcactttcggcggagggaccaaggtggagatcaaacgaactgtggctgcaccatctgtcttc

SEQ ID NO: 364 - protein, 101 aa, Homo sapiens
GERATLSCRASQSVSTYLAWYQQKAGQPPRLLIHDASKRATGIPARFSGSGSGTDFTLTISSLEPEDFAVYYCQQYGTSPLTFGGGTKVEIKRTVAAPSVF

SEQ ID NO: 365 - DNA, 303 bp, Homo sapiens
ggggaaagagccaccctctcctgcagggccagtcagagtattagtttccacttagcctggtaccagcagaaacctggccaggctcccagtctcctcatctacggaacatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggaccaaggtggacatcaaacgaactgtggctgcaccatctgtc

SEQ ID NO: 366 - protein, 101 aa, Homo sapiens
GERATLSCRASQSISFHLAWYQQKPGQAPSLLIYGTSTRATGIPARFSGSGSGTEFTLTISSLQSEDSAVYYCQQYHNWPPLTFGGGTKVDIKRTVAAPSV

SEQ ID NO: 367 - DNA, 303 bp, Homo sapiens
ggggaaagagccaccctctcctgcagggccagtcagagtattagtttccacttagcctggtaccagcagaaacctggccaggctcccaggctcctcatctatggggcatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggaccaaggtggacatcaaacgaactgtgactgcaccatctgtc

SEQ ID NO: 368 - protein, 101 aa, Homo sapiens
GERATLSCRASQSISFHLAWYQQKPGQAPRLLIYGASTRATGIPARFSGSGSGTEFTLTISSLQSEDSAVYYCQQYHNWPPLTFGGGTKVDIKRTVTAPSV

SEQ ID NO: 369 - DNA, 303 bp, Homo sapiens
ggggaaagagccaccctctcctgcagggccagtcagagtgtcagcagtagttacttagcctggtatcaacagagacctggccggtctcccaggctcctcatctatggtgcatccagcagggccactggcatcccagacaggttcagtggcagtgggtctgggacagacttcactctctccatcagcagactggagcctgaagattttgcagtgtatttttgtcagcagtatggcagcacaccgtacacttttggccaggggaccaaggtggagatgaaacgaactgtggctgcaccatctgtc

SEQ ID NO: 370 - protein, 101 aa, Homo sapiens
GERATLSCRASQSVSSSYLAWYQQRPGRSPRLLIYGASSRATGIPDRFSGSGSGTDFTLSISRLEPEDFAVYFCQQYGSTPYTFGQGTKVEMKRTVAAPSV

SEQ ID NO: 371 - DNA, 383 bp, Homo sapiens
ggcccaggactggtgaagccttgggagaccctgtccctcacctgcactgtctctggtggctccgtcagcagtagtcattactactggggctggatccgccagcccccagggacgggactggagtggattgggagtatcaattattatgggagcaccaactacaacccgtcccttaagagtcgcgtcaccatatccgtagacacgtccaggaaccagttctccctgaagctgaactctctgaccgccgcagacacggctgtatattactgtacgagacatgttgaggattgtcctagttccggctgctactcttactactactacttctacatggacgtctggggcaaagggaccacggtcaccgtctcctcagcctccaccaa

SEQ ID NO: 372 - protein, 127 aa, Homo sapiens
GPGLVKPWETLSLTCTVSGGSVSSSHYYWGWIRQPPGTGLEWIGSINYYGSTNYNPSLKSRVTISVDTSRNQFSLKLNSLTAADTAVYYCTRHVEDCPSSGCYSYYYYFYMDVWGKGTTVTVSSAST

SEQ ID NO: 373 - DNA, 383 bp, Homo sapiens
ggcccaggactggtgaagccttgggagaccctgtccctcacctgcactgtctctggtggctccgtcagcagtagtcattactactggggctggatccgccagcccccagggacgggactggagtggattgggagtatcaattattatgggagcaccaactacaacccgtcccttaagagtcgcgtcaccatatccgtagacacgtccaggaaccagttctccctgaagctgaactctctgaccgccacagacacggctgtatattactgtacgagacatgttgaggattgtcctagttccggctgctactcttactactactacttctacatggacgtctggggcaaagggaccacggtcaccgtctcctcagcctccaccaa

SEQ ID NO: 374 - protein, 127 aa, Homo sapiens
GPGLVKPWETLSLTCTVSGGSVSSSHYYWGWIRQPPGTGLEWIGSINYYGSTNYNPSLKSRVTISVDTSRNQFSLKLNSLTATDTAVYYCTRHVEDCPSSGCYSYYYYFYMDVWGKGTTVTVSSAST

SEQ ID NO: 375 - DNA, 365 bp, Homo sapiens
gggggaggcgtggtccaacctggggggtcccttagactctcctgtgcagcgtctggattcatcttcgatacctatggcatgcactgggtccgccaggctccaggcaagggactggagtgggtggcggttatctggtttgatggaagtaatcaatactatgcagagtccgtggagggccgattcaccatctccagagacaattccaggaatacactgtatctgcaaatgaacagcctgaaagacgaggatacggctatttattactgtgcgagaatgggattttgtagtggtcccagttgctatgcccaatactttcagcattggggccagggcaccctggtcaccgtctcctcagcctccaccaa

SEQ ID NO: 376 - protein, 121 aa, Homo sapiens
GGGVVQPGGSLRLSCAASGFIFDTYGMHWVRQAPGKGLEWVAVIWFDGSNQYYAESVEGRFTISRDNSRNTLYLQMNSLKDEDTAIYYCARMGFCSGPSCYAQYFQHWGQGTLVTVSSAST

SEQ ID NO: 377 - DNA, 365 bp, Homo sapiens
gggggaggcttggtccagcctggggggtccctgaaactctcctgtgcagcctctgggttcaccttcagtgggtctactatacactgggtccgccaggcttccgggaaagggctggagtgggttggccgtattagagtcaaagctgtcggctacgagacaacatatgctgcgtcggtgaagggcaggttcaccatttccagagatgactcacagaacacggcgtttctggaaatgcacagcctgaaaaccgaggacacggccgtgtatttttgtactggctatggttcggggactaccgacaactactacggtatggacgtctggggccaagggaccacggtcaccgtctcctcagcctccaccaa

SEQ ID NO: 378 - protein, 121 aa, Homo sapiens
GGGLVQPGGSLKLSCAASGFTFSGSTIHWVRQASGKGLEWVGRIRVKAVGYETTYAASVKGRFTISRDDSQNTAFLEMHSLKTEDTAVYFCTGYGSGTTDNYYGMDVWGQGTTVTVSSAST

SEQ ID NO: 379 - DNA, 350 bp, Homo sapiens
gggggaaggctggcacagcccggggggtcgctgaggctctcgtgtgtggtatctggagtaaactccttcagcagttatagtatgcattgggttcgccaggctccagggaagggccttgagtgggtctccttcatcactggtagcggtcgaaccgttaagtacgcagccgctttggagggccgattcactatctccagagacaatgacaagaaatcactttatttgcaattgagcagcctgagaggcgaggacacggctgtatattactgtgtgacagattcgctgaagacattggtggggcccacgtggggccagggaaccctggtcaccgtctcctcagcttgcaccaa

SEQ ID NO: 380 - protein, 116 aa, Homo sapiens
GGRLAQPGGSLRLSCVVSGVNSFSSYSMHWVRQAPGKGLEWVSFITGSGRTVKYAAALEGRFTISRDNDKKSLYLQLSSLRGEDTAVYYCVTDSLKTLVGPTWGQGTLVTVSSACT

SEQ ID NO: 381 - DNA, 350 bp, Homo sapiens
gggggaaggctggcacagcccggggggtcgctgaggctctcgtgtgtggtatctggagtaaactccttcagcagttatagtatgcattgggttcgccaggctccagggaagggccttgagtgggtctccttcatcactggtagcggtcgaaccgttaagtacgcagccgctttggagggccgattcactatctccagagacaatgacaagaaatcactttatttgcaattgagcagcctgagaggcgaggacacggctgtatattactgtgtgacagattcgctgaagacattggtggggcccacgtggggccagggaaccctggtcaccgtctcctcagcttccaccaa

SEQ ID NO: 382 - protein, 116 aa, Homo sapiens
GGRLAQPGGSLRLSCVVSGVNSFSSYSMHWVRQAPGKGLEWVSFITGSGRTVKYAAALEGRFTISRDNDKKSLYLQLSSLRGEDTAVYYCVTDSLKTLVGPTWGQGTLVTVSSAST

SEQ ID NO: 383 - DNA, 350 bp, Homo sapiens
tgggggaggctggcacagcccggggggtcgctgaggctctcgtgtgtggtatctggagtaaactccttcagcagttatagtatgcattgggttcgccaggctccagggaagggccttgagtgggtctccttcatcactggtagcggtcgaaccgttaagtacgcagccgctttggtgggccgattcactatctccagagacaatgacaagaaatcactttatttgcaattgagcagcctgagaggcgaggacacggctgtatattactgtgtgacagattcgctgaagacattggtggggcccacgtggggccagggaaccctggtcaccgtctcctcagcttccaccaa

SEQ ID NO: 384 - protein, 116 aa, Homo sapiens
WGRLAQPGGSLRLSCVVSGVNSFSSYSMHWVRQAPGKGLEWVSFITGSGRTVKYAAALVGRFTISRDNDKKSLYLQLSSLRGEDTAVYYCVTDSLKTLVGPTWGQGTLVTVSSAST

SEQ ID NO: 385 - DNA, 333 bp, Homo sapiens
gccatggccgagctcacccagtctccatcctccctgtctgcatctatgggagacagagtcaccatcacttgccgggcaagtcagaccattagcatatatttaaattggtatcagcagaaaccagggaaaccccctaaactcctgatctattctgcatcccgtttgcaaagtggggtcccatcaaggttcactggcagtggatctgggacagatttcactctcaccatcagcagtctgcatcctgaagattttgcaacttactactgtcaacagagttacagtagcttcataaccttcggccaagggacacgactggacttttacgaactgtgg

SEQ ID NO: 386 - protein, 111 aa, Homo sapiens
AMAELTQSPSSLSASMGDRVTITCRASQTISIYLNWYQQKPGKPPKLLIYSASRLQSGVPSRFTGSGSGTDFTLTISSLHPEDFATYYCQQSYSSFITFGQGTRLDFYELW

SEQ ID NO: 387 - DNA, 333 bp, Homo sapiens
gccatggccgagctcactcagtctccactccccctgcccgtcacccctggagagccggcctccatctcctgcaggtctagtcagagcctcctccatagtaatggatataattatttggattggtacctgcagaagccagggcagtctccacacctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcgaagttttggccaggggaccaagctggag

SEQ ID NO: 388 - protein, 111 aa, Homo sapiens
AMAELTQSPLPLPVTPGEPASISCRSSQSLLHSNGYNYLDWYLQKPGQSPHLLIYLGSNRASGVPDRFSGSGSGTDFTLKISRVEAEDVGVYYCMQALQTPRSFGQGTKLE

SEQ ID NO: 389 - DNA, 334 bp, Homo sapiens
gccatggccgagctcacacagtctccaggcaccctgtctttgtctccaggggaaagagccaccctctcctgcagggccagccagactattagcaacaactacttagcctggtaccagcagaaagttggccaggctcccaggctcctcatctatggtgcatccagcagggccactggcatcccagacaggttcagtggcagtgggtctgggtcggacttcactctcaccatcagcagactggagcctgaagattttgcagtgtattactgtcagcagtatggtacctcaccttcaaggacgttcggcccagggaccaaggtggaaatcaaacgat

SEQ ID NO: 390 - protein, 111 aa, Homo sapiens
AMAELTQSPGTLSLSPGERATLSCRASQTISNNYLAWYQQKVGQAPRLLIYGASSRATGIPDRFSGSGSGSDFTLTISRLEPEDFAVYYCQQYGTSPSRTFGPGTKVEIKR

SEQ ID NO: 391 - protein, 12 aa, Homo sapiens
TMGFTAPRFPHY

SEQ ID NO: 392 - protein, 12 aa, Homo sapiens
MQSPFTPHFAER

SEQ ID NO: 393 - protein, 12 aa, Homo sapiens
MQSPIVLPLSLS

SEQ ID NO: 394 - protein, 12 aa, Homo sapiens
HHEPGAWLPLSP

SEQ ID NO: 395 - DNA, 458 bp, Homo sapiens
gggggaggcttggcacagcctggggggtccctgagactctcctgtgcagcctctggattcacctttagcagccatggcatgagctgggtccgccaggctccagggaaggggctggagtgggtctcagctattagtgggagtggtggtaacacttactacgcagactccgtgaagggccggttcaccatctccagagacatttccaagaacacgctgtatctgcaaatgaacagcctgagagccgaagacacggccctatattactgtgcgagagggggcgcctatggttcggggagttataagtactggggccagggaaccctggtctccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 396 - protein, 152 aa, Homo sapiens
GGGLAQPGGSLRLSCAASGFTFSSHGMSWVRQAPGKGLEWVSAISGSGGNTYYADSVKGRFTISRDISKNTLYLQMNSLRAEDTALYYCARGGAYGSGSYKYWGQGTLVSVSSASTKGPSVFPLAPSSKSTSGGTAALGCLVKDYFPEPVTV

SEQ ID NO: 397 - DNA, 458 bp, Homo sapiens
gggggaggcttggcacagcctggggggtccctgagactctcctgtgcagcctctggattcacctttagcagccatggcatgagctgggtccgccaggctccagggaaggggctggagtgggtctcagctattagtgggagtggtggtaacacttactacgcagactccgtgaagggccggttcaccatctccagagacatttccaagaacacgctgtatctgcaaatgaacagcctgagagccgaagacacggccctatattactgtgcgagagggggcgcctatggttcggggagttataagtactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 398 - protein, 152 aa, Homo sapiens
GGGLAQPGGSLRLSCAASGFTFSSHGMSWVRQAPGKGLEWVSAISGSGGNTYYADSVKGRFTISRDISKNTLYLQMNSLRAEDTALYYCARGGAYGSGSYKYWGQGTLVTVSSASTKGPSVFPLAPSSKSTSGGTAALGCLVKDYFPEPVTV

SEQ ID NO: 399 - DNA, 455 bp, Homo sapiens
gggggaggcgtggtccagcctgggaggtccctgagactctcctgtgcagcctctggattcaccttcagtagctatgacatgcactgggtccgccaggctccaggcaaggggctggagtgggtggcaattatattggatgatggaagtaataaatactatgcagcctccgtgaagggccgattcaccatctccagagacaacgccaagaactccctgcatctgcaaatgagcagcctgagagccgaggacacggccctatattactgtgcgagagacctcccatatgctagtggccttgactactggggacagggaaccctggtcaccgtctcgtcaccctccaccaagggcccatcggtcttccccctggcaccctcctccaagagcacctctgggggcacagcggccctgggctgcctggtcaaggactacttccccgaaccggtgacggtgtc

SEQ ID NO: 400 - protein, 151 aa, Homo sapiens
GGGVVQPGRSLRLSCAASGFTFSSYDMHWVRQAPGKGLEWVAIILDDGSNKYYAASVKGRFTISRDNAKNSLHLQMSSLRAEDTALYYCARDLPYASGLDYWGQGTLVTVSSPSTKGPSVFPLAPSSKSTSGGTAALGCLVKDYFPEPVTV

SEQ ID NO: 401 - DNA, 473 bp, Homo sapiens
gggggaggcgtggtccagcctgggaggtccctgagactctcctgtgcagcctctggattcaccttcagtagctatgacatgcactgggtccgccaggctccaggcaaggggctggagtgggtggcaattatattggatgatggaagtaataaatactatgcagcctccgtgaagggccgattcaccatctccagagacaattccaagaacacgctgtatctgcaaatgaacagcctgagagctgaggacacggctgtgtattactgtgcgaaagtgcgaatagggaaggtcaataaggtcaataagtcctactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 402 - protein, 157 aa, Homo sapiens
GGGVVQPGRSLRLSCAASGFTFSSYDMHWVRQAPGKGLEWVAIILDDGSNKYYAASVKGRFTISRDNSKNTLYLQMNSLRAEDTAVYYCAKVRIGKVNKVNKSYFDSWGQGTLVTVSSASTKGPSVFPLAPSSKSTSGGTAALGCLVKDYFPEPVTV

SEQ ID NO: 403 - DNA, 473 bp, Homo sapiens
gggggaggcttggtcgagcctggagggtccctgagactctcctgtgaagccactggattcaccttcagtgactactacatgagttgggtccgccaggctcctgggaaggggctggaatggattgcatacattagtactggtagtagttacataaattatgcagactctaagaagggccgattcaccatctccagaaacaacgccaagaactcactgtatctgcaactgaacagcctgagagtcgacgacacggccgtgtattactgtgcgagatcgacacagagtttcggggagttattacccctcgtcctctttgaccactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 404 - protein, 157 aa, Homo sapiens
GGGLVEPGGSLRLSCEATGFTFSDYYMSWVRQAPGKGLEWIAYISTGSSYINYADSKKGRFTISRNNAKNSLYLQLNSLRVDDTAVYYCARSTQSFGELLPLVLFDHWGQGTLVTVSSASTKGPSVFPLAPSSKSTSGGTAALGCLVKDYFPEPVTV

SEQ ID NO: 405 - DNA, 464 bp, Homo sapiens
gggggaggcttggtacagcctggggggtccctgagactctcctgtgcagcctctggatccaccttaatcaactatgccatgagctgggtccgccaggctccagggaaggggctggagtgggtctcagttattagtggaactggtgttggcacatactacgcagactccgtgaggggccggttcaccatctccagagacgattccaacaacacggtggatctgcaaatgaatagcctgagagccgaggacacggccgtatattactgtgcgaaagatttccaagtcttcggtgactacatttctctaggctattggggccagggaatcctggtcaccgtcgcctcagcctccaccaagggcccatcggtcttccccctggcaccctcctccaagagcacctctgggggcacagcggccctgggctgcctggtcaaggactacttccccgaaccggtgacggtgtc

SEQ ID NO: 406 - protein, 154 aa, Homo sapiens
GGGLVQPGGSLRLSCAASGSTLINYAMSWVRQAPGKGLEWVSVISGTGVGTYYADSVRGRFTISRDDSNNTVDLQMNSLRAEDTAVYYCAKDFQVFGDYISLGYWGQGILVTVASASTKGPSVFPLAPSSKSTSGGTAALGCLVKDYFPEPVTV

SEQ ID NO: 407 - DNA, 455 bp, Homo sapiens
ggcccaggactggtgaagccttcggggaccctgtccctcacctgcgctgtctctggtggctccatcagcagtagtaactggtggatttgggtccgccagcccccagggaagaggctggagtggattggagaaatcgatcatagtgggactaccaactacaacccgtccctcaagagtcgagtcaccatgtcagtggtcaagtccaagaaccagttctccctgaagctgagctctgtgaccgccgcggacacggccgtctattactgtgcgagaggagcaaaggataactggggattcgactactggggccagggaatcttggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 408 - protein, 151 aa, Homo sapiens
GPGLVKPSGTLSLTCAVSGGSISSSNWWIWVRQPPGKRLEWIGEIDHSGTTNYNPSLKSRVTMSVVKSKNQFSLKLSSVTAADTAVYYCARGAKDNWGFDYWGQGILVTVSSASTKGPSVFPLAPSSKSTSGGTAALGCLVKDYFPEPVTV

SEQ ID NO: 409 - DNA, 455 bp, Homo sapiens
ggcccaggactggtgaagccttcggggaccctgtccctcacctgcgctgtctctggtggctccatcagcagtagtaactggtggattttggtccgccagcccccagggaagaggctggagtggattggagaaatcgatcatagtgggactaccaactacaacccgtccctcaagagtcgagtcaccatgtcagtggtcaagtccaagaaccagttctccctgaagctgagctctgtgaccgccgcggacacggccgtctattactgtgcgagaggagcaaaggataactggggattcgactactggggccagggaatcttggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 410 - protein, 151 aa, Homo sapiens
GPGLVKPSGTLSLTCAVSGGSISSSNWWILVRQPPGKRLEWIGEIDHSGTTNYNPSLKSRVTMSVVKSKNQFSLKLSSVTAADTAVYYCARGAKDNWGFDYWGQGILVTVSSASTKGPSVFPLAPSSKSTSGGTAALGCLVKDYFPEPVTV

SEQ ID NO: 411 - DNA, 455 bp, Homo sapiens
ggcccaggactggtgaagccttcggggaccctgtccctcacctgcgctgtctctggtggctccatcagcagtagtaactggtggatttgggtccgccagcccccagggaagaggctggagtggattggagaaatcgatcatagtgggactaccaactacaacccgtccctcaagagtcgagtcaccatgtcagtggtcaagtccaagaaccagttctccctgaagctgagctctgtgaccgccgcggacacggccgtctattactgtgcgagaggagcaaaggataactggggattcgactactggggccagggaaccctggtctccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 412 - protein, 151 aa, Homo sapiens
GPGLVKPSGTLSLTCAVSGGSISSSNWWIWVRQPPGKRLEWIGEIDHSGTTNYNPSLKSRVTMSVVKSKNQFSLKLSSVTAADTAVYYCARGAKDNWGFDYWGQGTLVSVSSASTKGPSVFPLAPSSKSTSGGTAALGCLVKDYFPEPVTV

SEQ ID NO: 413 - DNA, 470 bp, Homo sapiens
gggggaggcttggtacagcctggggggtccctgagactctcctgtgcagcctctggatccaccttaatcaactatgccatgagctgggtccgccaggctccagggaaggggctggagtgggtctcagttattagtggaactggtgttggcacatactacgcagactccgtgaggggccgattcaccatctccagagacaacgccaagaactcactgtctctgcaaacgaacagcctgagagccgaggacacggctgtctattattgcgttagattgtatagcagtggctgggacggctacttctatggactggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 414 - protein, 156 aa, Homo sapiens
GGGLVQPGGSLRLSCAASGSTLINYAMSWVRQAPGKGLEWVSVISGTGVGTYYADSVRGRFTISRDNAKNSLSLQTNSLRAEDTAVYYCVRLYSSGWDGYFYGLDVWGQGTTVTVSSASTKGPSVFPLAPSSKSTSGGTAALGCLVKDYFPEPVTV

SEQ ID NO: 415 - DNA, 440 bp, Homo sapiens
ggggctgaggtgaagaagactgggtcctcagtgaaggtgtcctgcatggtctccggaaacagcttcacccagcgtttcctgcactgggtgcgacaggcccccggacaagcgcttgagtggatggggtggatcacacctttcagtggaaatacctactacgcacagaaattccaggacagactcaccattacgggggacaggtctgtgagtacagcctacatggagttgagcagcctgagatctgacgacacagccatctattactgtgtgatttttggtcttgactactggggcaagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 416 - protein, 146 aa, Homo sapiens
GAEVKKTGSSVKVSCMVSGNSFTQRFLHWVRQAPGQALEWMGWITPFSGNTYYAQKFQDRLTITGDRSVSTAYMELSSLRSDDTAIYYCVIFGLDYWGKGTLVTVSSASTKGPSVFPLAPSSKSTSGGTAALGCLVKDYFPEPVTV

SEQ ID NO: 417 - DNA, 440 bp, Homo sapiens
ggggctgaggtgaagaagactgggtcctcagtgaaggtgtcctgcatggtctccggaaacagcttcacccagcgtttcctgcactgggtgcgacaggcccccggacaagcgcttgagtggatggggtggatcacacctttcagtggaaatacctactacgcacagaaattccaggacagactcaccattacgggggacaggtctgtgagtacagcctacatggagttgagcagcctgagatctgacgacacagccatctattactgtgtgattttcggtcttgactactggggcaagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 418 - protein, 146 aa, Homo sapiens; identical to SEQ ID NO: 416

SEQ ID NO: 419 - DNA, 476 bp, Homo sapiens
ggggctgactttaagaagcctggggcctcagcgagggtctcctgcaaggcatcgggatacaccttcaccaactactacttccactgggtacgacaggcccctggacaagggcttgagtggatgggaataatcaaccctgttggtgaaactagagtcaatacagagaagttccgggacagagtcaccatgaccagggacacgtccacgaacacagtctacatggacctgagcagcctgagatctgaggatacggccgtctattattgtgcgaggtcatattattatgcttcggggagtcctgaggaggacgatgcttttgatatctggggccaggggtcaatggtcatcgtctcttcagcctccaccaagggcccatcggtcttccccctggcaccctcctccaagagcacctctgggggcacagcggccctgggctgcctggtcaaggactacttccccgaaccggtgacggtgtc

SEQ ID NO: 420 - protein, 158 aa, Homo sapiens
GADFKKPGASARVSCKASGYTFTNYYFHWVRQAPGQGLEWMGIINPVGETRVNTEKFRDRVTMTRDTSTNTVYMDLSSLRSEDTAVYYCARSYYYASGSPEEDDAFDIWGQGSMVIVSSASTKGPSVFPLAPSSKSTSGGTAALGCLVKDYFPEPVTV

SEQ ID NO: 421 - DNA, 476 bp, Homo sapiens
ggggctgaggtgaggaagcctggggcctcagtgagggtttcctgcagggcatctgcatacaccctcacagactactatatgcactgggtgcgacagacccctggacaagggcttgaatggatgggaataatcaaccctagtggtggtagcacaacctacgcacagaagttccagggcagagtcaccatgaccagggacacgtccaccagcacagtctacatggacctgagcagcctgagatctgaagacacggccgtgtattactgtgctaggtctgactacggacacttcgtgcaacactcctacttctacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 422 - protein, 158 aa, Homo sapiens
GAEVRKPGASVRVSCRASAYTLTDYYMHWVRQTPGQGLEWMGIINPSGGSTTYAQKFQGRVTMTRDTSTSTVYMDLSSLRSEDTAVYYCARSDYGHFVQHSYFYGMDVWGQGTTVTVSSASTKGPSVFPLAPSSKSTSGGTAALGCLVKDYFPEPVTV

SEQ ID NO: 423 - DNA, 455 bp, Homo sapiens
gggggaggcttgctcaagccaggagggtccctgagactctcctgtgtagcctctggattcagcataagcgacttctacatgagttggatccgccaggctccagggaaaggactggagtggatctcatacctcagtggtggcagtacttacaggagccacgcagactctgggaagggccgattcaccatctccagagacaacgccaagaattcactgtttttgcaaatgagtagcctgggagtcgaggacacggccgtgtatttttgtgcgaggcatgtgggagtggcgactgcctttgatatctggggccaagggacagtggtcactgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 424 - protein, 151 aa, Homo sapiens
GGGLLKPGGSLRLSCVASGFSISDFYMSWIRQAPGKGLEWISYLSGGSTYRSHADSGKGRFTISRDNAKNSLFLQMSSLGVEDTAVYFCARHVGVATAFDIWGQGTVVTVSSASTKGPSVFPLAPSSKSTSGGTAALGCLVKDYFPEPVTV

SEQ ID NO: 425 - DNA, 452 bp, Homo sapiens
ggcccaggggtggtgaagccttcggagaccctgtccctcacctgcattgtctccggtgactccatgaccagttattactgggcctggctccggcagtcgtcagggaagggactggagtggattggatatgccttcaatacgaggaatgacgagtatagtccctccttcaggggtcgagccaccatatcggtggacgcgtccaagagtcaggtctccctgcacttgacctctgtgacctctgtggacacggccgtgtacttttgtgcgagactcccttactctatcaattactttgacttctggggccagggaaccgttgtcaccgtgtcctcagcctccaccaagggcccatcggtcttccccctggcaccctcctccaagagcacctctgggggcacagcggccctgggctgcctggtcaaggactacttccccgaaccggtgacggtgtc

SEQ ID NO: 426 - protein, 150 aa, Homo sapiens
GPGVVKPSETLSLTCIVSGDSMTSYYWAWLRQSSGKGLEWIGYAFNTRNDEYSPSFRGRATISVDASKSQVSLHLTSVTSVDTAVYFCARLPYSINYFDFWGQGTVVTVSSASTKGPSVFPLAPSSKSTSGGTAALGCLVKDYFPEPVTV

SEQ ID NO: 427 - DNA, 476 bp, Homo sapiens
ggaggaggcttggtcaagcctggcgggtccctgagactctcctgcacagcctctggattcactttcagtaacggctggatgagctgggtccgccaggctcctgggaaggggctggagtgggtcggccggattagaagcaaccccgacggtggcacaacagactacgctgcacccttcaaaggcagattcaccatctcaagagatgattcaaaaaatacattgtttctgcaagtgaccagcctgaaaaccgaggacacaggcgtctattactgcatcacagatcggggtgactggaagtggggggtccctagggacctcacctactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 428 - protein, 158 aa, Homo sapiens
GGGLVKPGGSLRLSCTASGFTFSNGWMSWVRQAPGKGLEWVGRIRSNPDGGTTDYAAPFKGRFTISRDDSKNTLFLQVTSLKTEDTGVYYCITDRGDWKWGVPRDLTYWGQGTLVTVSSASTKGPSVFPLAPSSKSTSGGTAALGCLVKDYFPEPVTV

SEQ ID NO: 429 - DNA, 392 bp, Homo sapiens
gccatgatcgagctcacccagtctccagactccctggctgtgtctctgggcgagagggccaccatcaactgcaagtccagccagagtattttatacagctccaacagtcagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcattattatagtactcctccgtgggcgttcggccaggggaccaaggtggaaatcaaacgaactgtggctgcaccatctgtcttcatcttcccgccatctgatga

SEQ ID NO: 430 - protein, 130 aa, Homo sapiens
AMIELTQSPDSLAVSLGERATINCKSSQSILYSSNSQNYLAWYQQKPGQPPKLLIYWASTRESGVPDRFSGSGSGTDFTLTISSLQAEDVAVYYCQHYYSTPPWAFGQGTKVEIKRTVAAPSVFIFPPSD

SEQ ID NO: 431 - DNA, 392 bp, Homo sapiens
gccatggccgagctcactcagtctccagactccctggctgtgtctctgggcgagagggccaccatcaactgcaagtccagccagagtgttttatacacctccaacaataggaaccacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcattattatagtactcctccgtgggcgttcggccaggggaccaaggtggatttcaaacgaactgtggctgcaccatctgtcttcatcttcccgccatctgatga

SEQ ID NO: 432 - protein, 130 aa, Homo sapiens
AMAELTQSPDSLAVSLGERATINCKSSQSVLYTSNNRNHLAWYQQKPGQPPKLLIYWASTRESGVPDRFSGSGSGTDFTLTISSLQAEDVAVYYCQHYYSTPPWAFGQGTKVDFKRTVAAPSVFIFPPSD

SEQ ID NO: 433 - DNA, 371 bp, Homo sapiens
gccatggccgagctcacgcagtctccagccatcctgtctttgtctccaggagagagagccaccctctcctgcggggccagtcagagtgttcccagcaacctcttagcctggtaccagcagagacctggcctggcgcccaggctcctcgtctatgattcttccagcagggccactggcatcccggacaggttcagtggcagtgggtctggaacagccttcactctcaccatcagcagaatggagcctgaagattttgcagtatattactgtcaacagtacggttactcacctctgacttttggccgggggaccagactggagttcaaacgaactgtggctgcaccatctgtcttcatctcccgccatctgag

SEQ ID NO: 434 - protein, 123 aa, Homo sapiens
AMAELTQSPAILSLSPGERATLSCGASQSVPSNLLAWYQQRPGLAPRLLVYDSSSRATGIPDRFSGSGSGTAFTLTISRMEPEDFAVYYCQQYGYSPLTFGRGTRLEFKRTVAAPSVFISRHL

SEQ ID NO: 435 - DNA, 315 bp, Homo sapiens
gccatggccgagctcacgcagtctccaggcaccctatctgtgtctccaggggatagagccaccctctcctgtagggccagtcagagtgtcgacagcaactacttagcctggttccagcagaaacctggccaggctcccaggctcctcatttatggtgcgtatagcagggccactggcatcccagacaggttcagtggcagtgggtctgggacagacttcactctcaccatcagcagactggagcctgaggattttgtcgtgtattactgtcagcagtatcttagcccgccgatcaccttcggccaagggacacga

SEQ ID NO: 436 - protein, 105 aa, Homo sapiens
AMAELTQSPGTLSVSPGDRATLSCRASQSVDSNYLAWFQQKPGQAPRLLIYGAYSRATGIPDRFSGSGSGTDFTLTISRLEPEDFVVYYCQQYLSPPITFGQGTR

SEQ ID NO: 437 - DNA, 374 bp, Homo sapiens
gccatggccgagctcacgcagtctccagacaccctgtctttgtctccaggggaaagagccaccctctcctgtagggccagtcagagtgtcgacagcaactacttagcctggttccagcagaagcctggccaggctcccaggctcctcatttatggtgcgtatagcagggccactggcatcccagacaggttcagtggcagtgggtctgggacagacttcactctcaccatcagcagactggagcctgaggattttgtcgtgtattactgtcagcagtatcttagcccgccgatcaccttcggccaagggacacgactggagactaaacgaactgtggctgcaccatctgtcttcatcttcccgccatctgatga

SEQ ID NO: 438 - protein, 124 aa, Homo sapiens
AMAELTQSPDTLSLSPGERATLSCRASQSVDSNYLAWFQQKPGQAPRLLIYGAYSRATGIPDRFSGSGSGTDFTLTISRLEPEDFVVYYCQQYLSPPITFGQGTRLETKRTVAAPSVFIFPPSD

SEQ ID NO: 439 - DNA, 374 bp, Homo sapiens
gccatggccgagctcacgcagtctccaggcaccctgtctttgtctccaggggaaacagtctccctctcctgcagggccagtcagactgttctcagcaattacttagcctggtaccagcagaaacctggccaggctcccagggtcctcctctatggtgcatctagcagggccactggcatcccagacaggttcagtggcggtgggtctgggacagacttcactctaaccatcagcagactggagcctgaagattttgcagtgtattactgtcagcaatatgttagttcaccgtggacgttcggccaagggaccaaggtggaattcaaacgaactgtggctgcaccatctgtcttcatcttcccgccatctgatga

SEQ ID NO: 440 - protein, 124 aa, Homo sapiens
AMAELTQSPGTLSLSPGETVSLSCRASQTVLSNYLAWYQQKPGQAPRVLLYGASSRATGIPDRFSGGGSGTDFTLTISRLEPEDFAVYYCQQYVSSPWTFGQGTKVEFKRTVAAPSVFIFPPSD

SEQ ID NO: 441 - DNA, 374 bp, Homo sapiens
gccatggccgagctcacgcagtctccaggcaccctgtctttgtctccaggggaaagagccaccctctcctgcagggccagtcagagcattcgcagcaacttcttagcctggtaccagcagaaacctggccaggctcccaggctcctcatctttggtgcatcgaacagggccactggcatcccagacaggttcagtggcagtgggtctgggacagacttcactctcaccatcagtagactggagcctgaagattttgcggtttattactgtcagcagtatagtagctcaccggacacttttggccaggggaccaagctggagatcaaacgaactgtggctgcaccatctgtcttcatcttcccgccatctgatga

SEQ ID NO: 442 - protein, 124 aa, Homo sapiens
AMAELTQSPGTLSLSPGERATLSCRASQSIRSNFLAWYQQKPGQAPRLLIFGASNRATGIPDRFSGSGSGTDFTLTISRLEPEDFAVYYCQQYSSSPDTFGQGTKLEIKRTVAAPSVFIFPPSD

SEQ ID NO: 443 - DNA, 377 bp, Homo sapiens
gccatggccgagctcacgcagtctccaggcaccctgtctttgtctccaggggaaagagtcaccctctcctgcagggccagccagagcgttagtagcaactacttaacctggtaccagcagaaacctggccaggctcccaggctcctcatctatggtgcatccagaagggccactggcatcccagacaggttcagtggcagtgggtctgggaccgacttcactctcaccataagcagactggagcctgaagattttgcagtttattactgtcaacattatggtagctcacctccattccctttcggccctgggaccaaagtggatgtcaaacgaactgtggctgcaccatctgtcttcatcttcccgccatctgatga

SEQ ID NO: 444 - protein, 125 aa, Homo sapiens
AMAELTQSPGTLSLSPGERVTLSCRASQSVSSNYLTWYQQKPGQAPRLLIYGASRRATGIPDRFSGSGSGTDFTLTISRLEPEDFAVYYCQHYGSSPPFPFGPGTKVDVKRTVAAPSVFIFPPSD

SEQ ID NO: 445 - DNA, 368 bp, Homo sapiens
gccatggccgagctcacacagtctccagacaccctgtctctgccaccaggggaaagggccaccctctcttgcagggccagtgagagtattgatgggagacgcttggcctggtaccagcagcagcctggccaggctcccaggctcctcatttatgatgtttccaggagggccattggcgtcccatacaggttcagaggcagtgggtctgggacagacttcactctcaccatcggtggactggagcctgaagattttgcagtctactactgtcaacattatggtttctcagtgtacactttggccaggggaccaggctgcgtcccacgaactgtggctgcaccatctgtcttcatcttcccgccatctga

SEQ ID NO: 446 - protein, 122 aa, Homo sapiens
AMAELTQSPDTLSLPPGERATLSCRASESIDGRRLAWYQQQPGQAPRLLIYDVSRRAIGVPYRFRGSGSGTDFTLTIGGLEPEDFAVYYCQHYGFSVYTLARGPGCVPRTVAAPSVFIFPPS

SEQ ID NO: 447 - DNA, 371 bp, Homo sapiens
gccatggccgagctcacccagtctccatcctccctgtctgcatctgtaggagacagagtcaccatcacttgccgggcaagtcaggacattagaaatgatttaagctggtatcagcagaaaccagggagagcccctaatctcctgatctatggtgcatccagtttacagaggggggtcccatttaggttcagcggcagtggatctggctcagatttcactctcaccatcagcagcctgcagcctgaagattttgcaacttattactgtctacaagatcacaattaccctctaacgttcggccaggggaccagggtggaaatcaaacgaactgtggctgcaccatctgtcttcatcttcccgccatctgatga

SEQ ID NO: 448 - protein, 123 aa, Homo sapiens
AMAELTQSPSSLSASVGDRVTITCRASQDIRNDLSWYQQKPGRAPNLLIYGASSLQRGVPFRFSGSGSGSDFTLTISSLQPEDFATYYCLQDHNYPLTFGQGTRVEIKRTVAAPSVFIFPPSD

SEQ ID NO: 449 - DNA, 371 bp, Homo sapiens
gccatggccgagctcacccagtctccatcctccctgtctgcatctgtaggagacagagtcaccgtcacttgccgggcaagtcagaccattgccaactatttaaattggtatcagcaaaaaccagggaaagcccctaacctcctgatccaagctgcttccactttgcaaggtggggtcccatcaaggttcagtggcagtcgatctgggacagatttcactctcaccatcaccagtctgcagcctgaggattttgcaacttacttctgtcaacagagtttcagcgccccctggacgttcggccaagggaccaaagtggaaatcaaacgaactgtggctgcaccatctgtcttcatcttcccgccatctgatga

SEQ ID NO: 450 - protein, 123 aa, Homo sapiens
AMAELTQSPSSLSASVGDRVTVTCRASQTIANYLNWYQQKPGKAPNLLIQAASTLQGGVPSRFSGSRSGTDFTLTITSLQPEDFATYFCQQSFSAPWTFGQGTKVEIKRTVAAPSVFIFPPSD

SEQ ID NO: 451 - DNA, 309 bp, Homo sapiens
gccatggccgagctcacccagtctccatcctccctgtctgcatctgttggagacagagtcaccatcacttgccggtcaagtcagaacattaacatctacttaagttggtatcaacagaaaccagggagagcccctaaactcctgatctatgctacatccaatttgcaaagtggggtcccatcaaggttcagtggcagtggatctgggacagacttcactctcaccatcagcagtctgcaacctgaagattttgcaacttactactgtcaacagagttacagtgacccgacgttcggccaagggaccaag

SEQ ID NO: 452 - protein, 103 aa, Homo sapiens
AMAELTQSPSSLSASVGDRVTITCRSSQNINIYLSWYQQKPGRAPKLLIYATSNLQSGVPSRFSGSGSGTDFTLTISSLQPEDFATYYCQQSYSDPTFGQGTK

SEQ ID NO: 453 - DNA, 311 bp, Homo sapiens
accctcacctgccgggcaagtctgagcattagttactttttaaattggtatcagcagaaaccaggtaaagcccctaagctcctgatctatgctgcatcccgtttgcacagtggggtcccatcaaggttcagtggcagtgggtctgggacagaattcactctcaccatcagcagtttgcaacctgaagatcttgcaacttactactgtcaacagagttacggtactcctgggactttcggccctgggaccaaagcggccttcaaacgaactgtggctgcaccatctgtcttcatcttcccgccatctgatga

SEQ ID NO: 454 - protein, 103 aa, Homo sapiens
TLTCRASLSISYFLNWYQQKPGKAPKLLIYAASRLHSGVPSRFSGSGSGTEFTLTISSLQPEDLATYYCQQSYGTPGTFGPGTKAAFKRTVAAPSVFIFPPSD

(19) United States
(12) Patent Application Publication
(10) Pub. No.: US 2010/0316563 A1
(43) Pub. Date: Dec. 16, 2010

(54) Method For The Preparation Of New Oligoclonal Antibodies

(75) Inventors: Massimo Clementi, Milano (IT); Roberto Burioni, Milano (IT)

Correspondence Address: BRACCO RESEARCH USA INC., 305- COLLEGE ROAD EAST, PRINCETON, NJ, 08540, US

(73) Assignee: BRACCO IMAGING S.P.A., Milano (IT)

(21) Appl. No.: 12/679,109

(22) PCT Filed: Sep. 18, 2008
(86) PCT No.: PCT/EP08/62408

§ 371 Date: Aug. 27, 2010

(30) Foreign Application Priority Data
Sep. 20, 2007 (EP) 07116856.1
Jul. 18, 2008 (EP) 08160692.3

(51) Int. Cl. A61K 51/00; A61K 39/395; G01N 33/53; C12P 21/06; C12N 5/10; C12N 1/16; C12N 15/63; G01N 33/544; C07K 14/435; C07K 16/18; C07H 21/04

(52) U.S. Cl. 424/1.11; 424/133.1; 435/7.1; 435/69.6; 435/348; 435/366; 435/419; 435/254.2; 435/320.1; 436/528; 530/350; 530/387.3; 530/387.9; 536/23.5

(57) ABSTRACT

A process is provided for the preparation of antibodies or fragments thereof using a prokaryotic host cell containing DNA sequences encoding for said antibodies of fragments thereof, wherein said DNA sequence is derived from a coronary plaque sample. Compositions containing said antibodies are also provided. Ligands to said antibodies and compositions containing said ligands are also described.

The present invention relates to a method for preparing new oligoclonal antibodies, the antibodies themselves as well as fragments thereof and their uses as well as the antigen and ligands thereof. In particular, the present invention encompasses antibodies or fragments thereof that are directed against antigens possibly found in the coronary plaque. The present invention further relates to the nucleotidic sequences coding for these antibodies and amino acidic sequences of the antibodies or fragments thereof for use in immunoassays, as well as to the ligands of these antibodies or fragments thereof. Further, the invention encompasses diagnostic and therapeutic applications related to the use of said antibodies or fragments thereof or of their ligands.

### BACKGROUND

The acute coronary syndrome (also shortly referred to as ACS) is the manifestation of a plaque rupture in a coronary artery.

The rupture or the erosion of an atherosclerotic plaque, with the subsequent formation of thrombus and occlusion of the artery may cause myocardial infarction and unstable angina (see, for a general reference, “New insights into atherosclerotic plaque rupture” D. M. Braganza and M. R. Bennett, Postgrad. Med. J. 2001; 77; 94-98).

An atherosclerotic event begins as a subendothelial accumulation of lipid laden, monocytes derived foam cells and associated T cells which form a non-stenotic fatty streak. With progression, the lesion takes the form of an acellular core of cholesterol esters, bounded by an endothelialised fibrous cap containing smooth muscle cells (VMSC) and inflammatory cells (both macrophages and T lymphocytes). Also presented in the advanced lesions are new blood vessels and deposits of calcium hydroxyapatite may also appear in advanced lesions (see as a general reference, “Coronary disease: Atherogenesis: current understanding of the causes of atheroma” Peter L. Weissberg, Heart 2000; 83; 247-252).

The extracellular lipid core of the plaque is composed of free cholesterol, cholesterol crystals and cholesterol esters derived from lipids infiltrated the arterial wall or derived from the dead foam cells. The lipid core may affect the plaque by causing stress to the shoulder regions of the plaque; in addition, the lipid core contains prothrombotic oxidized lipids and it is further impregnated with tissue factors derived from macrophages in which the lipid core materials are highly thrombogenic when exposed to circulating blood (see, for instance, “Mechanism of Plaque Vulnerability and Rupture” Prediman K. Shah, Journal of the American College of Cardiology 2003).

The stability of the plaque depends also upon the vascular smooth muscle cells (SMCs) content of the plaque, as they are capable of synthesising the structurally important collagens types I and III. In contrast, macrophages and others inflammatory cells may release matrix metalloproteinases (MMPs) which degrade collagen and extracellular matrix, thus potentially weakening the plaque (see, “New insights into atherosclerotic plaque rupture” D. M. Braganza and M. R. Bennett, Postgrad. Med. J. 2001; 77; 94-98).

The structural components of the fibrous cap include matrix component such as collagen, elastin and proteoglycans, derived from SMCs. Said fibrous cap protects the deeper components of the plaque from contact with circulating blood and has been observed to thin out in the vicinity of the rupture (see, for example, “Mechanism of Plaque Vulnerability and Rupture” Prediman K. Shah, Journal of the American College of Cardiology 2003).

Ruptured plaques have been shown to have several histomorphologic features with respect to intact plaques. Therefore, when they are present, they are thought to indicate vulnerability to plaque rupture (see, for instance, “Mechanism of Plaque Vulnerability and Rupture” Prediman K. Shah, Journal of the American College of Cardiology 2003).

One of possible causes inherent to the plaque formation is thought to be caused by repeated injury to endothelium caused by the four “major” risk factors: smoking, hypertension, diabetes and hyperlipidaemia (high level of LDL and low level of HDL). Endothelial dysfunction following injury, moreover, plays a crucial role in plaque initiation, as lipids may pass more easily from the bloodstream into the tunica intima.

The rupture of a vulnerable plaque may occur either spontaneously, i.e. without occurrence of any of the above mentioned triggers or following a particular event, such as an extreme physical activity, a severe emotional trauma and stresses of different nature or acute infection.

Plaque rupture often leads to thrombosis with clinical manifestations of an ACS. The thrombotic response to a plaque rupture is probably regulated by the thrombogenicity of the constituents exposed on the plaque; generally, the plaque rupture develops in a lesion with a necrotic core and an overlying thin fibrous cap heavily infiltrated by inflammatory cells. A luminal thrombus further develops due to the physical contact between platelets and the necrotic core (see, for example, “Pathologic assessment of the vulnerable human coronary plaque” F. D. Kolodgie et al. Heart 2004; 90; 1385-1391).

Rupture or erosion of the fibrous cap exposes the highly thrombogenic collagenous matrix and lipid core to circulation leading inevitably to platelet accumulation and activation. This in turn leads to fibrin deposition and thrombus formation which may result into vessel occlusion, the latter being not inevitable, such as in the case of silent episodes (see, for instance, “Coronary disease: Atherogenesis: current understanding of the causes of atheroma” Peter L. Weissberg, Heart 2000; 83; 247-252).

Until recently, atherosclerosis was thought of as a degenerative and slowly progressive disease causing symptoms through its mechanical effects on blood flow, while it is now understood to be a dynamic inflammatory process that is eminently modifiable. Recent researches on cellular and molecular events underlying development and progression of atherosclerosis, focus the attention on the dynamic interaction between the plaque components that dictates the outcome of the disease (see, as a general reference “Coronary disease: Atherogenesis: current understanding of the causes of atheroma” Peter L. Weissberg, Heart 2000; 83; 247-252).

There are contrasting data for a relation between coronary syndrome and several pathogens to be assessed.

In a prospective study (see, for example, “Impact of viral and bacterial infectious burden on long term prognosis in patients with coronary artery disease” Rupprecht H. J. et al., Circulation 2001 Jul. 3; 104(1): 25-31) it was described the relation between stroke and 8 different pathogens (Herpes simplex virus 1-2, Epstein-Barr, Cytomegalovirus, Haemophilus influenzae, Mycoplasma pneumoniae, Helicobacter pylori and Chlamydia pneumoniae) in a group of 1018 patients; there was found an increase in mortality, related to the serum positivity for six to eight pathogens of 7% and 12.6% respectively.

De Palma and his group (“Patients with Acute Coronary Syndrome Show Oligoclonal T-Cell Recruitment Within Unstable Plaque” De Palma et al. Circulation 2006, 113: 640-646) conducted a study on the T cells repertoire recovered from blood sample and also directly from the coronary plaque of patients with acute coronary syndrome.

Antibody Structures

There exist five types of antibodies (also called immunoglobulins): IgG, IgA, IgD, IgM and IgE. The structure of IgG, depicted in FIG. 1, comprises two light chains of a molecular weight of approximately 23 KDa and two heavy chains of about 53-70 KDa. The four chains being linked to each other by disulfide bonds in a “Y” configuration.

Heavy chains are classified as γ, η, α, δ and ε with some subclasses among them, while light chains are classified as either κ or λ.

Each heavy chain comprises a constant region and a variable region, the latter being located at the N-terminal end of the immunoglobulin molecule of approximately 100 amino acids in length.

In particular, the most variable part of the immunoglobulin (Ig) heavy and light chains is the third complementarity-determining region (CDR3), a short amino acid sequence which is formed by the junctions between the V-D-J gene segments. CDR3 is found in the variable domains of antigen receptor (e.g. immunoglobulin and T cell receptor) protein that complements an antigen.

The variability of the CDR3 portion is responsible of the elevated number of antibodies produced and which are specific for any antigens; said variability is determined by the rearrangement of the V, D and J minigenes that occurs in the bone marrow during the generation of mature B cells.

After this first rearrangement has occurred, when the mature B cell encounters an antigen, further hypermutational events are responsible for the increased affinity of the antibody for that specific antigen.

“Lineage trees” or “dendrograms” have frequently been drawn to illustrate diversification, via somatic hypermutation of immunoglubulin variable region genes. More in particularly, the generation of lineage trees to visualize the lineage relationships of B cells mutant in the germinal centers has been used in the past to confirm the role of the germinal center as the location of somatic hypermutation and affinity maturation.

### Examples of Objects Encompassed by the Present Invention

The following are illustrative examples of the objects of the invention, that will be more apparent from the teaching of the whole disclosure.

A first object of the invention includes the isolated polynucleotidic sequences coding for the heavy chains of the antibodies and corresponding to the odd-numbered Sequence ID from 1 to 51, 65 to 105, 191 to 209, 253 to 295, 345 to 349, 371 to 383 and 395 to 427.

A second object of the invention is thus represented by the amino acidic sequences coding for the heavy chains of the antibodies and corresponding to the even-numbered Sequence ID from 2 to 52, 66 to 106, 192 to 210, 254 to 296, 346 to 350, 372 to 384 and 396 to 428.

A third object of the invention are the isolated polynucleotidic molecules coding for the light chains of antibodies and corresponding to the odd-numbered Sequence ID from 53 to 63, 107 to 189, 211 to 251, 297 to 343, 351 to 369, 385 to 389 and from 429 to 453.

As a forth object of the invention is thus represented by the amino acidic sequences coding for the light chains of antibodies and corresponding to the even-numbered Sequence ID from 54 to 64, 108 to 190, 212 to 252, 298 to 344, 352 to 370, 386 to 390 and from 430 to 454.

A fifth object of the present invention includes an expression vector, comprising one or more of the isolated polynucleotidic molecules, as well as the complementary sequences thereof, encoding for the amino acidic sequences corresponding to the even-numbered Sequence ID from 2 to 390 and from 396 to 454 and the homologous sequences thereof.

An additional object of the present invention includes an expression system comprising one or more of the isolated expression vector of the invention and a suitable host cell.

As further object of the present invention, there is provided a host cell comprising one or more of the expression vector of the present invention.

An additional object of the present invention includes a process for the production of recombinant antibodies or fragments thereof including the use of the expression system of the invention comprising one or more of the isolated polynucleotidic molecules comprising the odd-numbered Sequence ID from 1 to 389 and from 395 to 453 as well as the complementary and homologous sequences thereof.

A further object of the invention encompasses the isolated recombinant antibodies or fragments thereof produced by the host cell comprising the expression vector of the present invention.

It is another object of the present invention an immunoassay including the use of one or more of the amino acidic sequences corresponding to the even-numbered Sequence ID from 2 to 390 and from 396 to 454 and the homologous sequences thereof.

In an additional embodiment of the invention, there is provided a therapeutic composition comprising the antibodies of the present invention or any fragments thereof and a therapeutic moiety linked thereto.

In a further embodiment of the invention, there is provided a diagnostic composition comprising the antibodies of the invention or fragments thereof linked to a diagnostic moiety.

It is a still further embodiment of the present invention a ligand that specifically binds at least one of the antibodies of the invention or to any fragments thereof.

A further object of the invention, a method for the screening of molecules for identifying those having the most binding affinity for the antibodies of the present invention or for any fragments thereof.

As an additional embodiment of the present invention there is an immunoassay, which includes the use of the ligand identified according to the present invention.

In a still additional embodiment of the invention, it is disclosed a therapeutic or diagnostic composition comprising the ligand of the present invention, covalently linked or otherwise functionally associated to a therapeutic or to a diagnostic moiety or entity.

An additional embodiment of the invention is represented by the use of immunosuppressant, immunomodulant or antinfective agents for the preparation of pharmaceutical compositions for the treatment of coronary diseases, such as the acute coronary syndrome or of immuno-related pathologies.

In a further embodiment of the invention, there is provided a method for the identification of the ethiologic agent responsible for the development of immuno-related pathologies.

An additional embodiment of this invention is an amino acid consensus sequence of a putative ligand possibly found in the coronary plaque.

In a further embodiment of the invention, there are provided four peptides showing the consensus sequence.

### BRIEF DESCRIPTION OF THE FIGURES

FIG. 1 is a schematic representation of the structure of an IgG antibody molecule and of a Fab fragment thereof.

FIG. 2 represents the recombinant pattern for the production of antibodies.

FIG. 3 represents the number of functional gene segments in human immunoglobulin loci.

FIG. 4 is a schematic representation of the preparation of the antibodies or fragments thereof according to the present invention.

FIG. 5 shows the analysis of the VDJ and VJ gene for the heavy chains of the coronary plaque sample.

FIG. 6a graphically shows the homology percentage of light chains of peripheral blood samples compared to coronary plaque samples.

FIG. 6b graphically shows the homology percentage of heavy chains of peripheral blood samples compared to coronary plaque samples.

FIG. 7 shows the nucleotide sequence alignment of two clonal variants of heavy chain from a plaque (#8 e #24).

FIG. 8 shows the amino acid sequence alignment of two clonal variants of light chain from a plaque (#8 e #15).

FIG. 9 shows the alignment of the aminoacidic sequence of β-globin (as internal control) and standard β-globin L48931.

FIG. 10 shows the sequences of the primers used according to the present invention. A: the primers annealing to the 5′ of variable regions of K light chains; B: primers annealing to the 3′ of constant region of K light chains; C: primers annealing to the 5′ of variable regions of heavy chains; D: primers annealing to the 3′ of constant regions.

FIG. 11 is a schematic representation of a lineage tree.

FIG. 12 is a mutational lineage tree of clonally related groups of light chains

FIG. 13 is a mutational lineage tree of clonally related groups of heavy chains.

FIG. 14 shows the ELISA results for Fab 24 on Hep-2 cell lysate.

FIG. 15 shows the ELISA results for Fab 24 on syntetic ligands.

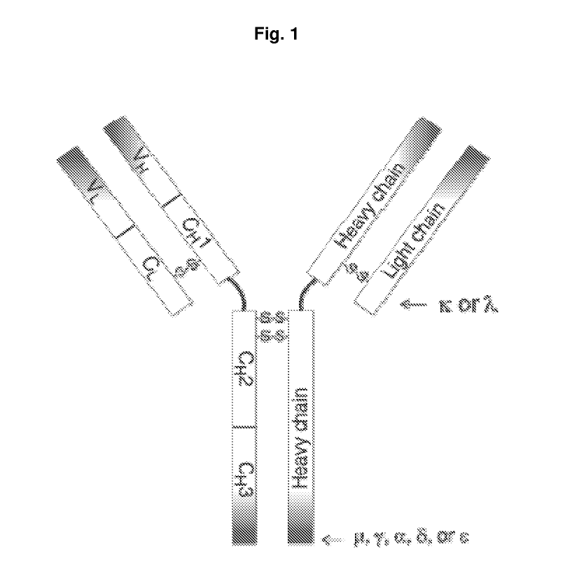

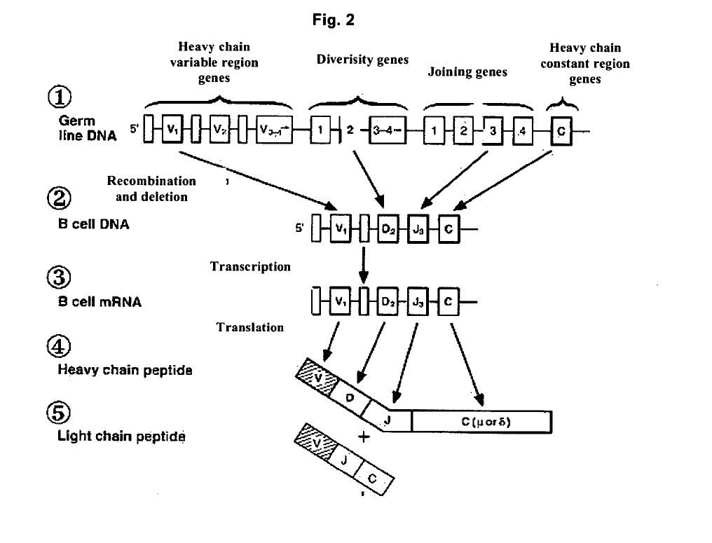

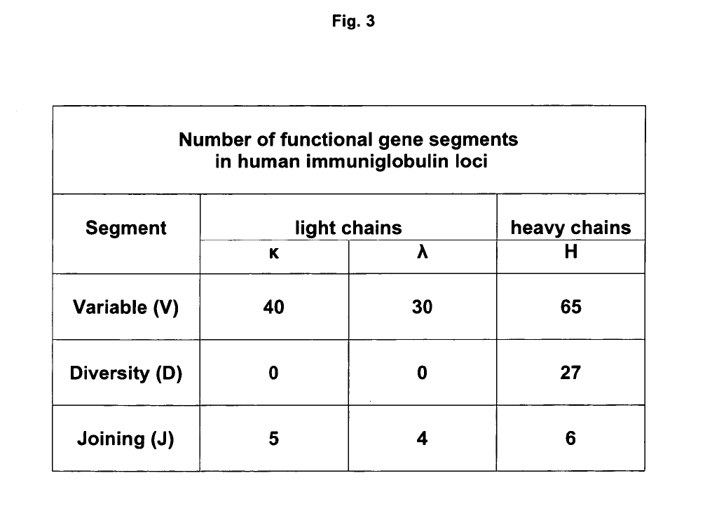

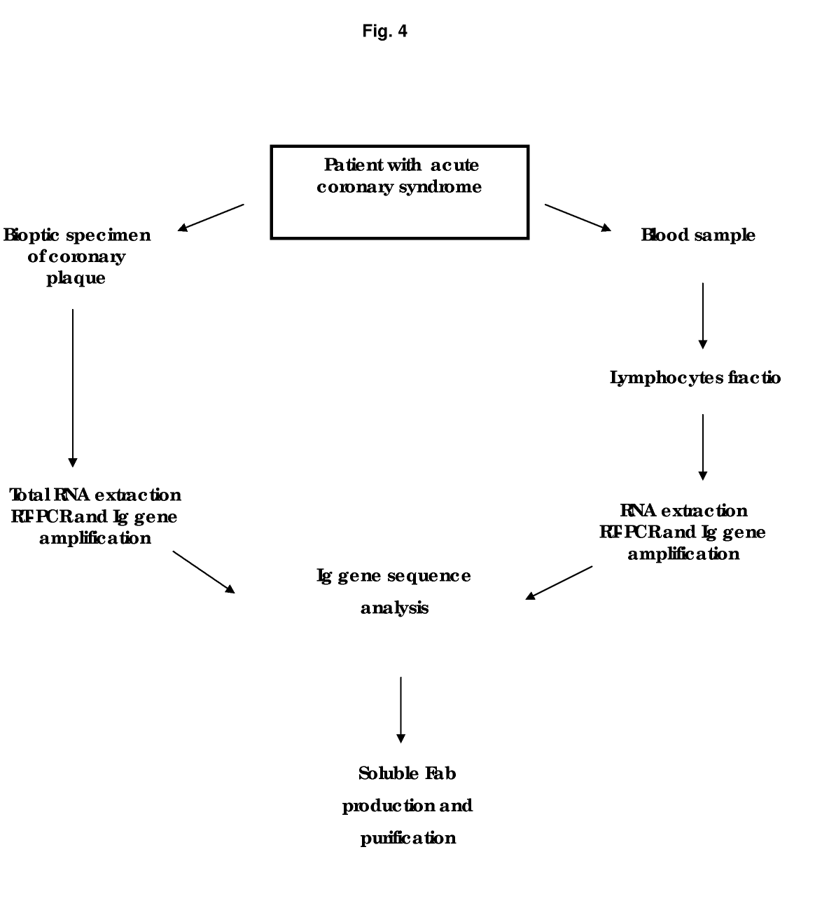

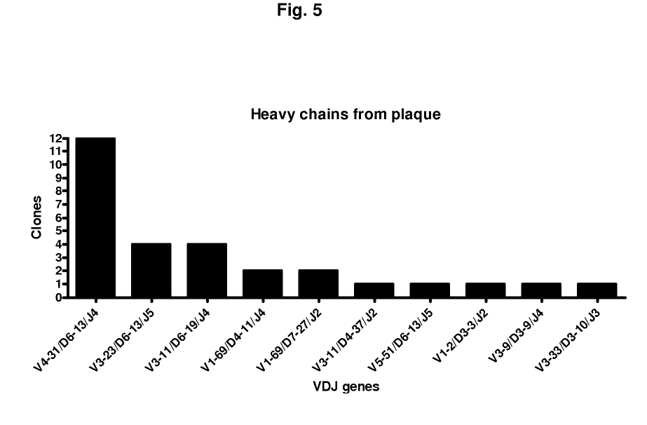

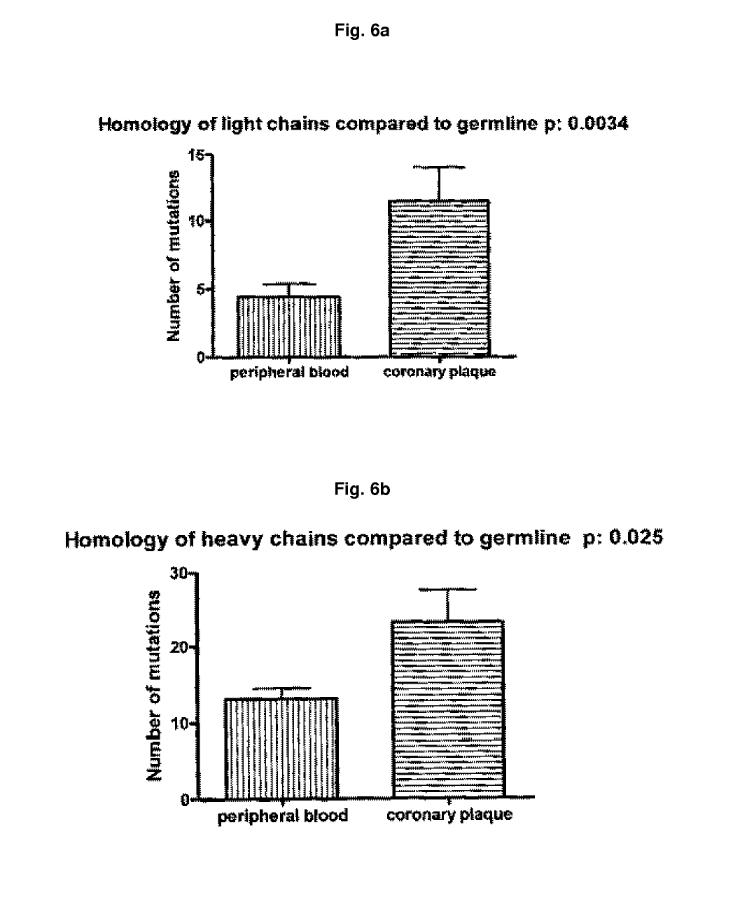

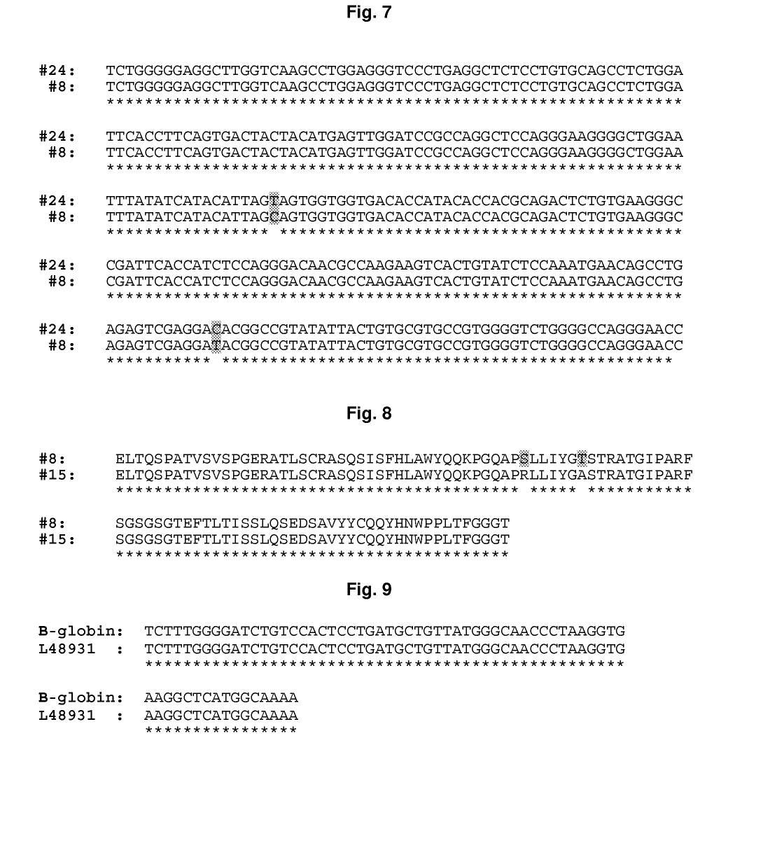

### DESCRIPTION OF THE INVENTION

Definitions

In the present invention, and unless otherwise provided, the term “isolated polynucleotide” or “isolated nucleotide” refers to a polynucleotide molecule, wherein polynucleotidic and nucleotidic, respectively, and polynucleotide and nucleotide are used alternatively with the same meaning, which is substantially free of any other cellular material or component that normally is present or interact with it in its naturally occurring environment, such as fragments of other nucleotidic or polynucleotidic sequences, proteins or other cellular component.

Unless otherwise provided, “complementary sequence” refers to the sequence which hybridizes to the sequence of interest under stringent conditions, resulting in two hydrogen bonds formed between adenine and tymine residues or three hydrogen bonds formed between cytosine and guanidine residues, respectively, and conservative analogs thereof having degenerative codon substitution or silent substitution, i.e. substitution of one or two or three consecutive nucleotides resulting in the same amino acid being coded due to the degeneracy of the genetic code.

The isolated polynucleotides within the meaning of the present invention, comprise, for instance, gene or gene fragments, exons, introns, mRNA, tRNA, rRNA, rybozyme, cDNA, plasmids, vectors, isolated DNA, probes and primers.

Unless otherwise indicated, the isolated polynucleotides of the invention, in addition to the specific ones described above, also comprise the complementary sequences thereto.

“cDNA” refers to the complementary DNA sequence, both single and double stranded and to any homologous sequence thereto and any fragment thereof, which codes continuously for an amino acidic sequence, i.e. its sequence is deprived of introns, and may be synthesized from isolated mRNA by retro-transcription techniques.

“Homologous sequence” within the meaning of the present invention refers to any sequence which is partially or almost identical to the sequence of interest; therefore, “homology” or “identity” of two or more sequences, comes from the factual measurement of the number of the same units, being those units nucleotides or amino acids, out of the total units componing said nucleotidic/amino acidic sequence, which occupy the same position. For example, 90% homology means that 90 of every 100 units making up a sequence are identical when the two sequences are aligned for maximum matching. Within the present invention, homologous sequences have an identity of at least 85%, preferably of 90%, more preferably of 95% and even more preferably of at least 99.5%.

“Conservative substitutions” of an amino is intended to be a substitution of an amino acid with another amino acid having the same properties, so that the substitution has no impact on the overall characterizing properties or functions of the peptide.

Examples of such conservative substitutions include the substitution of an amino acid with another amino acid belonging to the same group as follows:
(i) amino acids bearing a charged group, comprising Glutamine and Aspartic acid, Lysine, Arginine and Histidine;
(ii) amino acids bearing a positively-charged group, comprising Lysine, Arginine and Histidine;
(iii) amino acids bearing negatively-charged group, comprising Glutamine and Aspartic acid;
(iv) amino acids bearing an aromatic group, comprising Phenylalanine, Tyrosine and Tryptophan;
(v) amino acids bearing a nitrogen ring group, comprising Histidine and Tryptophan;
(vi) amino acids bearing a large aliphatic nonpolar group, comprising Valine, Leucine and Isoleucine;
(vii) amino acids bearing a slightly-polar group, comprising Metionine and Cysteine;
(viii) amino acids bearing a small-residue group, comprising Serine, Threonine, Aspartic acid, Asparagine, Glycine, Alanine, Glutamic acid, Glutamine and Proline;
(ix) amino acids bearing an aliphatic group comprising Valine, Leucine, Isoleucine, Metionine and Cysteine;
(x) amino acids bearing a small hydroxyl group comprising Serine and Threonine.

In the following disclosure, “CDR3” is a short sequence refers to the complementary-determining region, which is formed by the junctions between the V-D-J gene (in the heavy chain) or V-J gene (in the light chain) segments coding for an antibody. CDR3 is found in the variable domains that complements an antigen.

“Single clone” refers to a sequence coding for the CDR3 region of an antibody, which is able to specifically bind an antigen/epitope.

Sequences showing the same CDR3 are deemed to be produced by the same clone.

“Clonal variant” is intended to be any sequence, which differs by one or more nucleotide or amino acid, in presence of V region with identical mutations compared to the germline, identical VDJ or VJ gene usage, and identical D and J length.

“Replacement mutation” is intended to be a nucleotidic mutation which causes another amino acidic to be coded.

“Silent mutation” is intended to be a nucleotidic mutation which does not cause a change in the coded amino acid due to the degeneracy of the DNA.

An “expression vector” is intended to be any nucleotidic molecule used to transport genetic information.

An “isolated expression system” is intended to be a system for the expression of amino acidic molecules, and shall include one or more expression vectors comprising the nucleotidic sequences coding for one or more of the amino acidic molecules of the invention and a suitable host cell in which the one or more vectors are transfected.

“Host cell” as for the present invention is intended to be a cell comprising one or more expression vectors of the invention and which is capable of producing the corresponding coded amino acidic sequence or sequences or any fragments thereof, for example by expressing it on its surface.

“Antibodies” and “antibodies fragments” according to the present invention is intended to include whole antibodies, also referred to as immunoglobulin, of either type IgG, IgA, IgD, IgM or IgE, as well as any fragments thereof, such as proteolytic and/or recombinant fragments, like Fab fragments (produced upon digestion of Ig with papain), F(ab′)₂ (produced upon digestion of immunoglobulin with pepsin), Fab′, Fv, single chain antibodies (scFv) and single chain of antibodies, such as, for instance, heavy or light single chains.

“Ligand” within the present invention, is intended to be any agent that binds a recognized functional region of the antibody of the present invention or to any fragment thereof.

“Oligopeptide” according to the present invention is an amino acidic sequence comprising less than 50 amino acidic residues.

In the following description and unless otherwise provided, the “germline” sequence is intended to be the sequence coding for the antibody/immunoglobulin or of any fragment thereof deprived of mutations, therefore, the percentage of homology represents an indication of the mutational events which any type of heavy chain portion undergo after contact with an antigen; more in particular, said mutations involve the CDR3 portion of the antibody/immunoglobulin or of any fragment thereof.

The “R:S mutation” ratio refers to the ratio between replacing (R) and silent (S) mutations occurred in the FR or CDR3 portion of the antibody/immunoglobulin coding sequence.

Said ratio is higher for CDR3 than that of the FR sequence, as CDR3 undergoes an higher number of mutational event in order to adapt to the structure of the antigen. FR, in contrast, is a more conservative sequence, generally.

P-Value

“P-value” represents the significance of a mutational event.

In particular, the process of somatic hypermutation of rearranged V segments and the antigen selection of mutants with a higher affinity, allow the affinity maturation, in order to generate antibodies with improved binding properties to the antigen. This process leads to an accumulation of replacement mutations (R) in CDR regions, which are directly involved in the binding of antigen. On the contrary the silent mutations (S) accumulate in the FR regions, which are usually more conservative sequences in order to maintain the conformation of the antibody. In absence of the antigen selection, a random mutational process results in random distribution of R and S mutations in the sequence of both heavy and light chains of an antibody. However during the selection process, the R:S mutation ratio for CDR3 is usually higher than that of the FR sequence. Therefore, the p-value, which is calculated by multinomial distribution model that the excess (as for CDR) or the scarcity (as for FR) of mutations occurred by chance, relates to the probability of an antigen selection process. A low p-value indicates that there is a high probability that the variability of the heavy and light chains compared to the corresponding germline sequence, is due to the antigen-driven affinity maturation of the antibody.

A significant p-value is intended to be below 5%.

“Lineage trees” are a useful approach to study somatic hypermutation in B cells differentiation pathways by molecular analysis of antibodies genes expressed by clonally related cells.

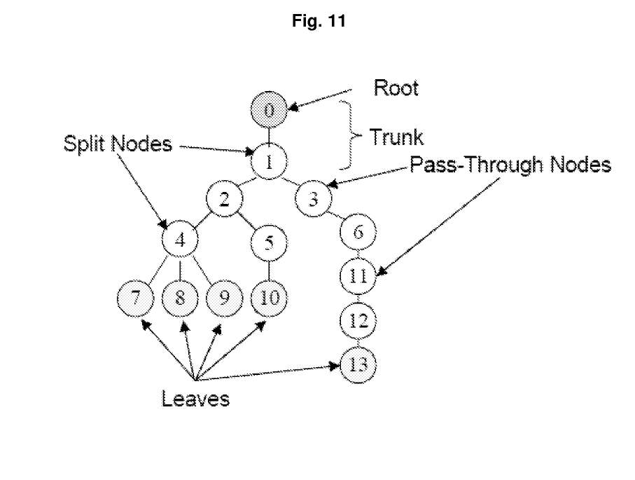

A lineage tree is defined, graphically, as a rooted tree where the nodes correspond to B cell receptor gene sequences (FIG. 11). For two nodes a and b it is said that b is a child of a if the sequence corresponding to b is a mutant of the sequence corresponding to a, which differs from b by at least one mutation and is one mutation further than b away from the original germline gene. Two B cells with identical receptors will correspond to the same node. Nodes in the tree can be either the root node, leaves (end-point sequences) or internal nodes, which can be either split nodes (branching points) or pass-through nodes.

Root is intended as representing the original B cell.

Leaves are intending to represent mutant B cells which were alive at the time of sampling and had no descendants at the time of observation.

Internal split nodes are intending as B cells that were produced during the maturation process and have more than one descendant.

Internal pass-through nodes refer to B cell with exactly one child.

Trunk is intended as the distance between the root to the first split node.

According to its first embodiment, the present invention concerns polynucleotidic molecules comprising any one of the sequences corresponding to the odd-numbered Sequence ID from 1 to 389 and from 395 to 453 and the complementary and homologous sequences thereto.

The polynucleotidic sequences of the present invention codes for the amino acidic sequences of antibodies or any fragments thereof which binds to an antigen or any fragment thereof possibly found in the coronary plaque.

Preferably, within the present invention, the isolated polynucleotides of the above first embodiment are cDNA molecules.

cDNA is obtained by retro-transcription from mRNA molecules according to the well-known procedures in the art.

According to the first object of the present invention, there are also provided amino acidic sequences corresponding to the even-numbered Sequence ID from 2 to 390 and from 396 to 454; as well as the homologous sequences thereof, and any sequences bearing conservative substitutions and fragments thereof.

As indicated, these definitions are intended to encompass analogous sequences, so as to include those sequences wherein, in the case of amino acid sequences, at least one or more amino acids are substituted by a derivative, such as the corresponding D-isomer or, for example, the corresponding sulphated, glycosylated or methylated amino acid; or one or more and up to 10% of the total amino acids making up a sequence may be substituted by a derivative thereof, such as, for example, cysteine may be substituted by homocysteine. There are also included sequences bearing conservative substitutions.

According to the present invention, there are also included the polynucleotidic sequences coding for antibodies or for any fragments thereof according to the first embodiment of the invention and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, as for the first object of the present invention, hypermutated amino acidic sequences are also encompassed.

Accordingly, there are also included the polynucleotidic sequences coding for the amino acidic sequences having a ρ-value of the CDR3 portion less than 5%, preferably less than 2%, more preferably less than 1% and even more preferably less than 1% and the coded amino acidic sequences thereof.

As set hereinbefore, according to the present invention, there is included the synthesis of cDNA molecules, which is performed from mRNA isolated from a suitable sample of the active coronary plaque of a patient.

For the purpose of the present invention, said suitable samples of the active coronary plaque includes a sample of the coronary plaque taken immediately after an infarction event, i.e. so-called “fresh-sample” or, alternatively, a sample may be taken and conserved under liquid nitrogen for a suitable period of time so as not to impair nor alter its histological properties and be further analysed.

For the purpose of the present invention, patients with acute coronary syndrome (ACS) have been selected, which have experienced a typical chest pain occurring less than 48 hours from hospital admission or ECG changes suggesting myocardial damage. In order to exclude possible confusing factors, patients with recent infectious diseases, immunologic disorders, immunosuppressive therapy or neoplastic diseases have been excluded.

Isolation of mRNA molecules from the above suitable samples, i.e. both from coronary plaque and peripheral blood, is carried out according to well-known methods. For a general reference, see, for instance Molecular cloning. Sambrook and Russell. Cold Spring Harbor Laboratory Press Cold Spring Harbor, N.Y. Third Edition 2001.

According to the second embodiment, the expression vector of the invention is selected from the group comprising for example, plasmid, cosmid, YAC, viral particle, or phage and comprises one or more of the polynucleotidic sequences according to the first embodiment of the invention; in a preferred aspect, the expression vector is a plasmid, comprising one or more of the polynucleotidic sequences according to the first embodiment of the invention.

In a most preferred embodiment of the invention, the expression vector, i.e. a plasmid, comprises one of more of the polynucleotidic sequences of the invention selected from the group comprising the odd-numbered Sequence ID from 1 to 389 and from 395 to 453.

Expression vectors ordinarily also include an origin of replication, an operably linked, i.e. connected thereto in such a way as to permit the expression of the nucleic acid sequence when introduced into a cell, promoter located upstream the coding sequences, together with a ribosome binding side, an RNA splice site, a polyadenylation site and a transcriptional sequence. The skilled artisan will be able to construct a proper expression vector and, therefore, any proper expression vector according to the selected host cell; for example, by selecting a promoter which is recognized by the host organism.

In an even more preferred embodiment, the expression vector of the present invention is represented by the vector described by Burioni et al. (Human Antibodies 2001; 10 (3-4): 149-54).

The isolated expression system according to the third embodiment of the invention may comprise a single expression vector, which comprises one or more of any one of the polynucleotidic sequences of the invention.

Alternatively, the above expression system may comprise two or more expression vectors, each of them comprising one or more of any one of the polynucleotidic molecules of the invention.

For example, an expression vector may comprise a polynucleotidic molecule of the invention coding for the light chain of an antibody or fragment thereof and a second expression vector may include a polynucleotidic molecule of the invention coding for the heavy chain of an antibody or fragment thereof.

In an embodiment of the invention, the expression system comprises a single expression vector including one or more of the polynucleotidic molecules comprising the odd-numbered Sequence ID from 1 to 389 and from 395 to 453 and coding for the amino acidic sequences and corresponded to the even-numbered Sequence ID from 2 to 390 and from 396 to 454 and any homologous sequence thereto.

In a preferred embodiment of the invention, the expression system comprises one expression vectors comprising the polynucleotidic sequences coding for a light chain, i.e. being selected from the sequences corresponding to the odd-numbered Sequence ID from 53 to 63, 107 to 189, 211 to 251, 297 to 343, 351 to 369, 385 to 389 and from 429 to 453 and a second polynucleotidic sequence coding for a heavy chain, i.e. being selected from the sequences corresponding to the odd-numbered Sequence ID from 1 to 51, 65 to 105, 191 to 209, 253 to 295, 345 to 349, 371 to 383 and from 395 to 427.

In a most preferred embodiment of the present invention, the expression system includes a vector comprising the polynucleotidic sequence coding for the light chain as set forth in Sequence ID n° 53 and the second vector comprising any one of the polynucleotidic sequences coding for the heavy chain as set forth in Sequence ID n° 21, 37, 43 and 51, respectively.

The preparation of the expression vector comprised into the expression system of the invention, includes the insertion of the appropriate nucleic acid molecule o molecules into one or more vector or vectors, which generally comprises one or more signal sequences, origins of replication, one or more marker genes o sequence, enhancer elements, promoters, and transcription termination sequences according to methods well-known in the art.

For a general reference to said procedure, see, for instance Phage display, Cold Spring Harbor Laboratory Press. Cold Spring Harbor, N.Y.

For instance, the sequences coding for the heavy chain of the present invention are inserted into the expression vector with a Flag o a six-Histidine tail, for being easily detectable.

The host cell according to a forth embodiment of the present may be, for instance, a prokaryotic cell or a eukaryotic cells.

Suitable prokaryotic cells include gram negative and gram positive and may include, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces. For example, publicly available strains which may be used are, for instance, E. coli K12 strain MM294 (ATCC 31,446); E. coli X1776 (ATCC 31,537); E. coli strain W3110 (ATCC 27,325) and K5 772 (ATCC 53,635) or E. coli XL1-Blue, which represents the preferred E. coli strain.

In addition to prokaryotes, eukaryotic microbes such as filamentous fungi or yeast are suitable host cells. Saccharomyces cerevisiae, also known as common baker's yeast, is commonly used; other yeast are, for instance, Saccharomyces, Pichia pastoris, or Kluyveromyces such as, for example, K. lactis, K. fragilis, K. bulgaricus, K. wickeramii, K. waltii, K. drosophilarum, K. thermotolerans, and K. marxianus, Schizosaccharomyces, such as Schizosaccharomyces pombe, yarrowia, Hansenula, Trichoderma reesia, Neurospora crassa, Schwanniomyces such as Schwanniomyces occidentalis, Neurospora, Penicillium, Tolypociadium, Aspergillus such as A. nidulans, Candida, Torulopsis and Rhodotorula.

In addition, suitable eukaryotic cells used for the preparation of the expression system may be derived from multicellular organisms as well, such as from invertebrate cells or plant cells. Plant cells include, for instance, Agrobacterium tumefaciens and Nicotiana tabacum. In addition, insect cells may be used, which include, for instance, Drosophila S2 and Spodoptera Sf9.

Conversely, mammalian host cell include Chinese hamster ovary (CHO) and COS cells. More specific examples further include monkey kidney CVI line transformed by SV40 (COS-7, ATCC CRL 1651); human embryonic kidney line, Chinese hamster ovary cells/-DHFR, mouse sertoli cells, human lung cells (W138, ATCC CCL 75); human liver cells (Hep G2, HB 8065); and mouse mammary tumor (MMT 060562, ATCC CCL51).

The selection of the appropriate host cell is deemed to be within the knowledge of the skilled person in the art, i.e. prokaryotic cells may be used for the preparation of antibodies fragments such as Fabs, while for the preparation of whole antibodies such as IgG, eukaryotic cells like yeasts may be employed.

Methods for cell transfection and transformation in order to prepare the above disclosed host cell comprising the above expression system depends upon the host cell used and are known to the ordinarily skilled artisan.

For example, treatments with calcium or electroporation are generally used for prokaryotes, while infection with Agrobacterium tumefaciens is used for transformation of certain plant cells. For mammalian cells, calcium phosphate precipitation may be used as disclosed by Graham and van der Eb, Virology, 52:456-457 (1978).

However, other methods for introducing polynucleotidic sequences into cells, such as, for example, nuclear microinjection, electroporation, bacterial fusion with intact cells, or polycations, may also be used.

Host cells, in addition, may also be transplanted into an animal so as to produce transgenic non-human animal useful for the preparation of humanized antibodies or fragments thereof. A preferred non-human animal includes, for instance, mouse, rat, rabbit, hamster.

The production of recombinant antibodies and fragments thereof as for the fifth embodiment of the invention is performed according to known methods in the art and includes the use of the isolated polynucleotidic sequences of the invention.

In particular, said method includes the steps of:
a) isolating mRNA from a suitable sample of the coronary plaque;
b) performing reverse transcription in order to obtain the corresponding cDNA;
c) preparing an expression system comprising the one or more cDNA molecule or molecules obtained from step b) and any one of the above disclosed suitable host cells;
d) culturing the host cell under suitable growth conditions;
e) recovering the produced antibodies or any fragments thereof; and
f) purifying said antibodies or any fragments thereof.

In particular, steps a) to f) are performed according to known methods in the art as it will be apparent from the following Examples.

In order to assess the influence on the results obtained by statistically occurring mutations or other mechanism different from those involved in the maturation of B-cells of the coronary plaque, cloning and sequencing is also performed on a small portion of a gene having a conserved region. Accordingly, as internal reference, β-globin gene is chosen; in particular, standard β-globin L48931 is used.

Therefore, it is a further object of the present invention, the isolated recombinant antibodies and fragments thereof produced by the host cell of the present invention and according to the method disclosed above, include immunoglobulin (shortly referred to as Ig) of the IgG type, while “fragments thereof” preferably include Fab fragments of IgG.

Preferably, the isolated recombinant antibodies fragments of IgG of the present invention comprise the amino acidic sequences set forth in Sequence ID n° 54 and, alternatively, any one of the amino acidic sequences set forth in Sequence ID n° 22, 44, 52 and 38.

According to the present invention, there are also included the amino acidic sequences coding for antibodies or for any fragments thereof which may be produced according to the process above disclosed and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, there are also included the amino acidic sequences having a ρ-value of the CDR3 portion less than 5%, preferably less than 2%, more preferably less than 1% and even more preferably less than 1%.

According to another object of the invention, there is provided an immunoassay, which comprises the use of the antibodies or of any fragments thereof produced according to the present invention.

Immunoassays are test based on the formation of an antigen/antibody complex and can be either competitive or non-competitive.

Competitive immunoassays include the testing of unknown samples containing a particular antigen which competes for the binding to the antibodies with another but labelled antibody; therefore, the response is inversely proportional to the concentration of the antigen in the unknown sample.

Conversely, non-competitive immunoassays, also called “sandwich assays”, include the use of an immobilized antibody, bound by an antigen, thus forming a complex which is targeted by a labelled antibody; the result of said methods is, therefore, directly proportional to the concentration of the antigen.

Widespread used immunoassays include, for example, RIA (Radio Immuno Assay), Western Blot, ELISA (Enzyme-linked Immunosorbent Assay), immunostaining, immunoprecipitation, immunoelectrophoresis, immunofluorescence, luminescent immunoassay (LIA), immunohystochemistry, which are routinely used in lab practise.

A preferred immunoassay according to the present invention is an ELISA test.

ELISA is a well-established biochemical technique, which allows the detection and further quantification of biomolecules, such as antibodies or fragments thereof, antigens, proteins, hormones and other organic molecules, in a given sample; preferably, according to the present invention, the above mentioned ELISA test is used for the detection of a specific antigen.

ELISA test, in particular, may include the use of two antibodies, one of which, the first antibody, is selective for the molecule of interest, i.e. the antigen, and it is immobilized onto an ELISA plate. A mixture possibly containing said molecule of interest is added, incubation for a suitable and sufficient time is allowed, then a first washing is performed in order to remove unbound material. The secondary antibody coupled to an enzyme and specific for the complex formed between the molecule of interest and the first antibody is further added. There follows a second step of washing of the ELISA plate and the addition of a chromogenic substrate. The resulting variation in colour may be assessed by spectrophotometric techniques and is directly related through a colorimetric standard curve to the quantity of the complex formed and thus to the concentration of the molecule of interest present in the sample.

Samples to be tested by the above immunoassay of the invention are, for example, samples of the unstable coronary plaque taken from patient immediately after an infarction event, i.e. a so-called “fresh” sample as said before, or a sample which has been conserved under liquid nitrogen after being taken; alternatively, it may consist of a sample of whole blood or serum.

The immunoassay test according to the present invention represents a valuable diagnostic tool, when included in programs for the screening of either the population at risk or not of developing acute coronary syndrome (ACS) or other coronary diseases.

As for an additional embodiment of the invention, there is disclosed a therapeutic composition comprising the antibodies or any fragments thereof of the present invention and a therapeutic moiety covalently linked thereto.

Said therapeutic composition is able to selectively target a therapeutic agent to the coronary plaque site.

Well-known advantages of said targeted composition include, among others, the possibility of reducing the quantity of active principle to be administered, thus reducing the potentially side effects thereof.

For said purpose, therapeutic moieties may include as non limiting examples, radionuclides, drugs, hormones, hormone antagonists, receptor antagonists, enzymes or proenzymes activated by another agent, autocrines or cytokines, antimicrobial agents; toxins can also be used.

Drugs and prodrugs are included as well.

A further embodiment of the invention relates to a diagnostic composition comprising the antibodies of the invention or any fragment thereof linked to a diagnostic moiety for the visualisation of the coronary plaque site.

The diagnostic compositions according to the present invention comprise the antibody or any fragments thereof, produced according to the present invention, covalently linked to at least one diagnostic moiety in order to selectively target the coronary plaque site and thus allowing its localization.

Therefore, it will be possible to precisely localise the site where the coronary plaque developed and to even better understand the extent of the occurred lesion to the vase. In addition, this represents a very useful tool before removal of the plaque by surgery.

Diagnostic moieties allow the detection by the visualising techniques used in the field of medicine, such as, for example, MRI (magnetic resonance imaging), CT (computer tomography), ultrasound, ecography, x-rays, and other diagnostic techniques within the knowledge of the skilled person in the art.

The kind of diagnostic moiety will be selected according to the diagnostic technique to be used.

According to a still further object of the present invention, there are provided ligands, that is to say, molecules which do bind selectively to the antibodies or to any fragments thereof.

The ligand or ligands of the present invention may also be an agent that binds any surface or internal sequences or conformational domains or any other part of the target antibody or fragments thereof. Therefore, the “ligands” of the present invention encompass agents that may have no apparent biological function, beyond their ability to bind the target of interest.

Accordingly, proteins, peptides, polysaccharides, glycoproteins, hormones, receptors, cell surfaces antigens, antibodies or fragments thereof such as Fab fragments, F(ab′)2, Fab′, Fv and single chain antibodies (scFv) or even anti-idiotype antibodies, toxins, viruses, substrates, metabolites, transition state analogs, cofactors, inhibitors, drugs, dyes, nutrients, growth factors, etc., without limitation, are included as well within the above definition.

In a preferred embodiment, the ligand of the present invention is an oligopeptide as above defined; preferably is a peptide comprising 4 to 12 amino acids, more preferably is a peptide comprising 4 to 10 and even more preferably is a peptide comprising 6 to 8 amino acids.

The identification of the ligands may be performed by screening tests on libraries of compounds. In particular, according to the present invention, said identification includes the use of the antibodies provided by the present invention or of any fragments thereof.

A method for the identification of ligands to the antibodies of the present disclosure or to any fragments thereof, therefore, represents a further object of the invention.

For instance, said method may include the binding of the antibodies or fragments thereof onto a solid phase, for example through a streptavidin-biotin linkage, followed by contacting the molecules to be tested with the thus prepared solid phase, so as to allow them binding to the complementary antibodies and then washing to remove unbounded material; finally, the extend of the binding can be determined by various methods such as, for instance, an ELISA test.

Preferably, said ELISA test is one wherein a first antibody or a fragment thereof, being selected from those of the present invention, is linked to a solid phase, for instance, by a biotin/streptavidin linkage, then a mixture containing the molecules to be tested is added, incubation is allowed for a suitable period of time, followed by removal of unbound material by washing. After that, the secondary antibody is admixed and incubation is allowed again. The molecules showing the highest affinity for the antibodies of the invention or for any fragments thereof may thus be isolated, identified and quantified according to well-known methods such as, for instance, by colorimetric measurements.

Alternatively, as for an additional embodiment of the present invention there is provided an immunoassay including the use of a ligand identified according to the present invention.

Said immunoassay may be any one of the immunoassays already mentioned above as for the second object of the invention.

For example, an immunoenzymatic test as for the claimed invention may be an immunohystologic assay as further detailed in Example 10.

The above immunohystologic assay can be performed in order to investigate the presence inside the plaque of the ligands identified and disclosed in the present invention according to the above embodiments.

In a still additional embodiment of the invention, there is disclosed a therapeutic composition comprising a ligand identified by the above method of the invention and covalently linked to a therapeutic moiety.

A therapeutic moiety for said purpose may be any one of those already described above.

In particular, the therapeutic composition thus provided may selectively target a therapeutic agent to the coronary plaque site.

There is also disclosed a diagnostic composition comprising a ligand identified by the above method of the invention and covalently linked to a diagnostic moiety.

A diagnostic moiety for said purpose may be any one of those already described above.

As for an additional embodiment of the invention, there is claimed the use of immunosuppressant compounds for the preparation of a pharmaceutical composition for the treatment of coronary diseases, such as the acute coronary syndrome (ACS) or of immuno-related pathologies.

Immuno-related pathologies include pathologies wherein the physiologic mechanisms triggering and controlling the immuno-responses are altered.

Immunosuppressant compounds may be selected from the group comprising by way of non limiting example, glucocorticoids, alkylating agents, antimetabolites, methotrexate, azathioprine and mercaptopurine, cytotoxic antibiotics such as dactinomycin, anthracyclines, mitomycin C, bleomycin, mithramycin, ciclosporine, interferons, opioids, TNF binding protein, mycophenolate, small biological agent; in addition, monoclonal and polyclonal antibodies are comprised.

In a further embodiment, the present invention provides for a method for the identification, demonstration and characterization of a local antigen-specific and antigen-driven stimulation of the immune system, providing useful details that can be used for the identification of the aetiopatology, for the definition of targets and for the design of immunotherapy and immunoprophylaxis.

In particular, said method includes the steps of testing the affinity of the antibodies of the present invention or of any fragments thereof for pathogenic agents potentially responsible for the development of the coronary disease.

With the aim of better understanding of the present invention, and without posing any limitation to it, the following Examples are given.

### Example 1

### Sample Collection

1a) Sampling of Atherosclerotic Coronary Plaque

A sufficient amount of tissue is obtained from an atherosclerotic plaque of a patient with acute coronary syndrome undergoing coronary atherectomy and it is stored in liquid nitrogen.

1b) Sampling of Peripheral Blood

5 ml of peripheral blood from the same patient from whom the tissue of Example 1a is taken, at the same time, and stored in tubes treated with EDTA.

### Example 2

### mRNA Extraction

2a) mRNA Extraction from Coronary Plaque

The plaque taken according to Example 1a is homogenized and the total mRNA is extracted according to conventional methodologies using a commercial kit for the extraction of mRNA (Rneasy kit, Qiagen, Germany) and according to the instructions provided by the manufacturer.

2b) mRNA Extraction from Peripheral Blood Sample

5 ml of the peripheral blood collected according to Example 1b is diluted in an equal volume of PBS (phosphate buffered saline) at 37° C., overlaid onto 15 ml of Histopaque-1077 (Sigma-Aldrich, St Louis, Mo.) and centrifuged at 300 g for 30 minutes at room temperature. Lymphocytes are collected at the interface using a Pasteur pipette, diluted in 15 ml of PBS and further centrifuged at 300 g. The obtained pellet is thus resuspended in 15 ml of PBS and a small aliquot is taken in order to count the cells using a counting chamber (Burker). Finally, the cell suspension is centrifuged at 300 g and mRNA extraction is performed on the obtained pellet according to the procedure described above.

### Example 3

### mRNA Retrotranscription

3a) Retrotranscription of mRNA from Coronary Plaque Sample

Reverse transcription of mRNA from the coronary plaque sample obtained as from Example 2a is performed using a commercial kit for the retrotranscription of mRNA, Superscript III RT (Sigma-Aldrich, St Louis, Mo.) according to the manufacturer's instruction. The cDNA synthesis is performed according to standard procedures from the total mRNA primed with oligo(dT).

3b) Retrotranscription of mRNA from Peripheral Blood Sample

The same procedure of Example 3a is performed on mRNA obtained according to Example 2b.

### Example 4

### Amplification of cDNA Sequences

4a) Amplification of cDNA Sequences from Coronary Plaque Sample

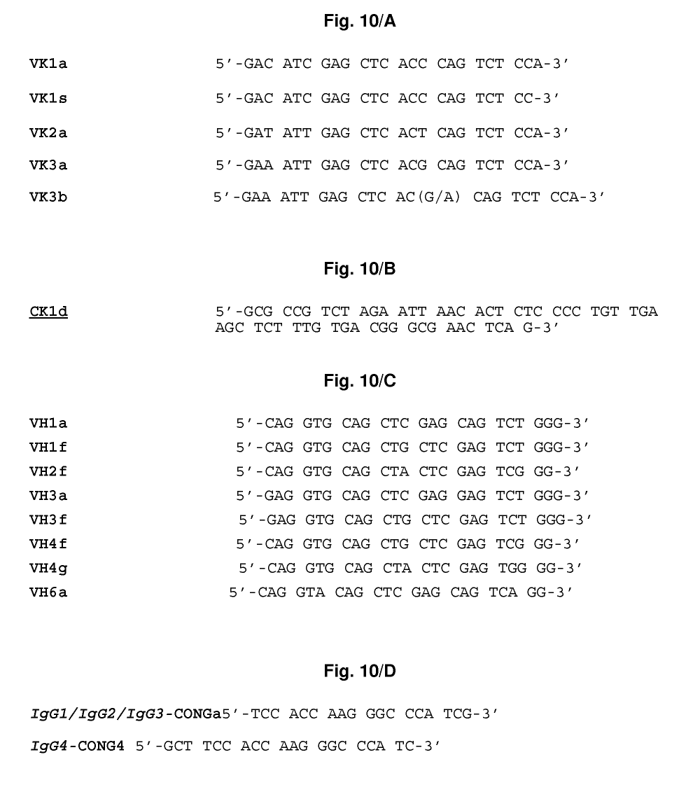

1 μl of cDNA obtained from the Example 3a undergo polymerase chain reaction. The reverse primers are designed in order to anneal to the segments of sequences coding for the constant region of heavy and light chains respectively (FIGS. 10B and D as for light and heavy chains, respectively). The forward primers are “family specific” and are designed to correspond to the 5′ end of the heavy and light chain genes in the first framework region FIGS. 10A and C as for light and heavy chains respectively); see, as a reference, Phage display, Cold Spring Harbor Laboratory Press. Cold Spring Harbor, N.Y. Third Edition 2001. For the heavy chains, primers specific for IgG1 and IgG2 isotypes are used, whereas for the light chains primers specific for K isotype are used. Amplification round is conducted with the following thermal profile: 94° C. for 15 seconds, 52° C. for 1 minute and 72° C. for 90 seconds. The reaction is conducted for 35 cycles. A negative control (the same mixture without DNA) and a positive control (a known sequence is inserted) are included in each reaction. The PCR product is analyzed by electrophoresis in a 2% agarose gel containing ethidium bromide. The reaction yields a ≅650 by band corresponding to the light chains, and a 700 by corresponding to the heavy chains. The amplicon, i.e. the product of the PRC process) is extracted from the gel with the use of a commercial kit for the extraction of DNA (QIAquick gel extraction kit; Qiagen, Germany) according to the manufacturer's instructions. Finally, the PCR products are recovered as per standard methods.

4b) Amplification of cDNA Sequences from Peripheral Blood Sample

The amplification of cDNA sequences from peripheral blood sample (cDNA obtained from Example 3b) is performed using the same procedure of Example 4a.

### Example 5

### Sequencing

The sequences obtained according to the previous Examples are sequenced in for quantitative and qualitative analysis.

5.1) Heavy and Light Chain Sample Processing

A sample of clones of heavy and light chains obtained from coronary plaque sample and from peripheral blood sample obtained according to the previous Examples 4a and 4b, respectively, is picked up in order to be sequenced by Big Dye chemistry and analyzed using ABI PRISM 3100 sequencer.

The obtained sequences are individually aligned to the germline segments using a database available in ImMunoGeneTics (available through the web site http://imgt.cines.fr), in order to identify the V,D,J and V and J genes recurrence as for the heavy and light chains respectively, the homology level with the germline and the extent of somatic mutations. CDR3 sequence identity is used for identifying the clones; as mentioned above, sequences with identical CDR3 are deemed to come from the same clone.

The polynucleotidic sequences from coronary plaque samples obtained according to the above Example 4a for the heavy chains correspond to the odd-numbered Sequence ID from 1 to 51, 65 to 105, 191 to 209, 253 to 295, 345 to 349, 371 to 383 and from 395 to 427 and code for the amino acidic sequences corresponding to the even-numbered Sequence ID from 2 to 52, 66 to 106, 192 to 210, 254 to 296, 346 to 350, 372 to 384 and from 396 to 428.

The polynucleotidic sequences from coronary plaque samples obtained according to the above Example 4a for the light chains correspond to the odd-numbered Sequence ID from 53 to 63, 107 to 189, 211 to 251, 297 to 343, 351 to 369, 385 to 389 and from 429 to 453 and code for the amino acidic sequences corresponding to the even-numbered Sequence ID from 54 to 64, 108 to 190, 212 to 252, 298 to 344, 352 to 370, 386 to 390 and from 430 to 454.

5.2) B-Globin Sequence: Internal Reference

The analysis of five clones shows that the obtained sequence of β-globin is identical to the sequence present in database (see FIG. 9, which reports one of the alignment with the standard β-globin L48931), thus demonstrating that no mutational event was due to the process variabilities.

5.3) Light Chains from Coronary Plaque Sample

The results of the sequencing of clones obtained from the coronary plaque samples according to Example 4a are shown in the following Table I for each clone V, D and J gene column report the type of sequence found to code for the V, D and J variable portion of the heavy chain, respectively. Homology percentage refers to the percentage of homology between each one of the sequence cloned from the coronary plaque sample and the sequence of the corresponding germline sequence as above disclosed.

TABLE I

| | | | Homology | R:S mutations | | p-value | |
|---|---|---|---|---|---|---|---|
| # Clone | VK gene | JK Gene | (%) | FR | CDR | FR | CDR |
| 1 | V3-15*01 | J4*01 | 96.86 | 1/0 | 4/2 | 0.00338 | 0.00063 |
| 2 | V3-15*01 | J4*01 | 96.07 | 2/1 | 4/2 | 0.00423 | 0.00207 |
| 7 | V3-15*01 | J4*01 | 96.07 | 2/1 | 4/2 | 0.00423 | 0.00207 |
| 15 | V3-15*01 | J4*01 | 96.07 | 2/1 | 4/2 | 0.00423 | 0.00207 |
| 24 | V3-15*01 | J4*01 | 96.07 | 2/1 | 4/2 | 0.00423 | 0.00207 |
| 25 | V3-15*01 | J4*01 | 96.07 | 2/1 | 4/2 | 0.00423 | 0.00207 |
| 38 | V3-15*01 | J4*01 | 96.07 | 2/1 | 4/2 | 0.00423 | 0.00207 |
| 67 | V3-15*01 | J4*01 | 96.07 | 2/1 | 4/2 | 0.00423 | 0.00207 |
| 86 | V3-15*01 | J4*01 | 96.07 | 2/1 | 4/2 | 0.00423 | 0.00207 |
| 36 | V3-15*01 | J4*01 | 96.07 | 2/1 | 4/2 | 0.00423 | 0.00207 |
| 8 | V3-15*01 | J4*01 | 94.90 | 3/2 | 5/2 | 0.00197 | 0.00091 |
| 32 | V3-15*01 | J4*01 | 95.31 | 3/2 | 4/2 | 0.00462 | 0.00489 |
| 10 | V3-15*01 | J4*01 | 94.90 | 3/2 | 5/2 | 0.00197 | 0.00091 |
| 29 | V3-15*01 | J4*01 | 94.90 | 3/2 | 5/2 | 0.00197 | 0.00091 |
| 39 | V3-15*01 | J4*01 | 94.90 | 3/2 | 5/2 | 0.00197 | 0.00091 |
| 9 | V3-15*01 | J4*01 | 95.22 | 2/1 | 5/3 | 0.00067 | 0.00061 |
| 28 | V3-15*01 | J4*01 | 95.45 | 4/2 | 1/3 | 0.045 | 0.3 |
| 51 | V3-20*01 | J4*01 | 86.77 | 24/4 | 2/1 | 0.89847 | 0.65339 |
| 52 | V3-20*01 | J4*01 | 86.77 | 24/4 | 2/1 | 0.89847 | 0.65339 |
| 57 | V3-20*01 | J4*01 | 86.77 | 24/4 | 2/1 | 0.89847 | 0.65339 |
| 63 | V3-20*01 | J4*01 | 86.77 | 24/4 | 2/1 | 0.89847 | 0.65339 |
| 49 | V1-33*01 | J4*01 | 94.9 | 3/4 | 3/0 | 0.00865 | 0.02608 |
| 53 | V1-33*01 | J4*01 | 94.9 | 3/4 | 3/0 | 0.00865 | 0.02608 |
| 56 | V1-33*01 | J4*01 | 94.9 | 3/4 | 3/0 | 0.00865 | 0.02608 |
| 64 | V1-33*01 | J4*01 | 94.9 | 3/4 | 3/0 | 0.00865 | 0.02608 |
| 29b | V3-11*01 | J2*01 | 95.68 | 3/2 | 2/0 | 0.10342 | 0.06186 |
| 58 | V5-2*01 | J1*01 | 97.25 | 5/0 | 1/0 | 0.74747 | 0.2472 |

5.4) Heavy Chains from Coronary Plaque Sample

The same procedure adopted for the analysis of the sequences of the light chains is repeated for the sequence of the heavy chains obtained according to Example 4a.

The results are shown in the following Table II.

TABLE II

| | | | | Homology | R:S mutations | | p-value | | |
|---|---|---|---|---|---|---|---|---|---|
| #Clone | V Gene | D Gene | J Gene | (%) | FR | CDR | FR | CDR | Isotype |
| 20(4) | V3-23*01 | D6-13*01 | J5*02 | 91.32 | 7/8 | 5/3 | 0.00131 | 0.11974 | IgG1 |
| 11(2) | V3-23*01 | D3-10*01 | J3*02 | 95.07 | 4/3 | 4/2 | 0.01368 | 0.04852 | IgG2 |
| 13(12) | V4-31*03 | D6-13*01 | J4*02 | 93.58 | 8/4 | 5/0 | 0.12581 | 0.04459 | IgG1 |
| 9(4) | V3-11*01 | D6-19*01 | J4*02 | 92.07 | 11/6 | 4/0 | 0 | 0.23027 | IgG1 |
| 22(2) | V1-69*01 | D4-11*01 | J4*02 | 71.37 | 49/5 | 18/1 | 0.75964 | 0.00235 | IgG1 |
| 1 | V3-23*01 | D3-16*01 | J4*02 | 98.11 | 4/0 | 1/0 | 0.76745 | 0.31544 | IgG1 |
| 5 | V3-13*01 | D4-17*01 | J2*01 | 91.18 | 14/2 | 7/1 | 0.33946 | 0.01352 | IgG1 |
| 2 | V3-15*07 | D2-21*01 | J6*02 | 80.74 | 42/7 | 2/1 | 0.99879 | 0.99128 | IgG1 |
| 19 | V3-33*01 | D3-10*01 | J3*02 | 92.91 | 10/5 | 4/0 | 0.24167 | 0.19402 | IgG1 |
| 26 | V3-33*01 | D3-3*01 | J6*02 | 93.93 | 8/3 | 5/0 | 0.19861 | 0.03969 | IgG1 |
| 25 | V3-9*01 | D3-9*01 | J4*02 | 93.96 | 6/2 | 6/2 | 0.02766 | 0.00928 | IgG1 |
| 4 | V3-23*01 | D7-27*01 | J5*02 | 97.73 | 5/1 | 0/0 | 0.83827 | 0.78322 | IgG1 |
| 6 | V5-51*03 | D6-13*01 | J5*01 | 83.01 | 20/16 | 8/1 | 0.00469 | 0.18476 | IgG1 |
| 23 | V1-69*01 | D1-7*01 | J4*02 | 86.74 | 19/9 | 6/1 | 0.14441 | 0.20547 | IgG1 |

5.5) Light Chains from Peripheral Blood Sample

The same procedure applied for the analysis of the light chain as above disclosed is repeated on the sequences of the light chains obtained from the peripheral blood sample obtained according to Example 4b.

The results are shown in the following Table III.

TABLE III

| | | | Homology | | |
|---|---|---|---|---|---|
| # Clone | VK Gene | JK Gene | (%) | R:S mutations | |
| 4a | V4-1*01 | J1*01 | 98.16 | 4/1 | 0/0 |
| 5a | V4-1*01 | J1*01 | 95.6 | 3/4 | 2/0 |
| 9a | V4-1*01 | J4*01 | 98.16 | 1/0 | 3/0 |
| 7 | V3-20*01 | J2*01 | 98.44 | 1/0 | 1/1 |
| 8a | V3-20*01 | J2*02 | 96.12 | 0/1 | 4/1 |
| 10a | V3-20*01 | J2*01 | 98.44 | 2/0 | 0/1 |
| 2 | V3-20*01 | J1*01 | 100 | 0/0 | 0/0 |
| 14a | V3-20*01 | J1*01 | 96.51 | 3/2 | 2/0 |
| 1 | V3-15*01 | J4*01 | 97.64 | 1/2 | 3/1 |
| 1a | V3-15*01 | J1*01 | 94.5 | | |
| 12a | V3-15*01 | J2*01 | 100 | 0/0 | 0/0 |
| 5 | V5-2*01 | J2*01 | 95.68 | 7/3 | 0/0 |
| 6 | VD1-13*01 | J4*01 | 97.25 | 3/0 | 1/0 |
| 6a | V1-33*01 | J5*01 | 100 | 0/0 | 0/0 |
| 9a | V4-1*01 | J4*01 | 98.16 | 1/0 | 3/0 |
| 7a | V1-5*03 | J2*02 | 96.48 | 4/1 | 1/1 |
| 8 | V1-39*01 | J2*01 | 100 | 0/0 | 0/0 |
| 11 | V1-39*01 | J4*01 | 95.29 | 5/5 | 1/0 |
| 15 | V1-39*01 | J1*01 | 100 | 0/0 | 0/0 |
| 16 | V1-6*01 | J2*01 | 96.07 | 5/1 | 2/0 |
| 19 | V2-30*01 | J2*01 | 91.85 | 6/5 | 3/1 |
| 14 | V3-11*01 | J4*01 | 100 | 0/0 | 0/0 |
| 13 | V2-30*01 | J1*01 | 96.29 | 2/2 | 2/0 |

5.6) Heavy Chains from Peripheral Blood Sample

The same procedure is repeated on the sequences of the heavy chains from the peripheral blood sample and the results are shown in the following Table IV.

TABLE IV

| | | | | Homology | R:S mutations | |
|---|---|---|---|---|---|---|
| # Clone | VH gene | DH gene | JH gene | (%) | FR | CDR |
| 5 | V4-59*02 | D6-25*01 | J3*02 | 88.93 | 13/6 | 7/3 |
| 8 | V3-48*01 | D3-3*01 | J4*02 | 93.18 | 7/7 | 4/0 |
| 12 | V3-23*01 | D3-10*01 | J3*01 | 91.69 | 11/2 | 7/2 |
| 14 | V4-34*01 | D2-2*01 | J6*02 | 96.94 | 1/4 | 3/0 |
| 18 | V4-34*01 | D3-22*01 | J1*01 | 96.18 | 6/2 | 2/0 |

Therefore, as clones 11, 9, 13 and 20 of the sequences amplified from the plaque show the highest divergence from the germline sequence, they are selected in order to be expressed together with the light chain 8.

5.7) Results

The above data show that both heavy and light chains from coronary plaque sample have an oligoclonal pattern and a characteristic VDJ and VJ gene pattern, respectively.

In addition, somatic hypermutations in the CDR3 portion are more frequent for the heavy and light chains of the coronary plaque sample compared to the peripheral blood sample; moreover, a higher number of mutational events occurred to the sequences of light and heavy chains from coronary plaque samples.

5.8) Mutational Lineage Tree

Lineage trees have been drawn for the sequences obtained according to the previous Examples aiming to illustrate diversification via somatic hypermutation of immunoglobulin variable-region (IGV) within clonally related groups of immunoglobulins.

5.8.1) Lineage Tree Generation

Germlines genes are identified according to Example 5. Tree bifurcations are identified by using a nj algorithm and the p model of evolution as implemented in the Mega 3 software (http://www.megasoftware.net/) using the germline sequence to root the tree. Manual corrections are performed to optimise the topology according to sequence visual inspection.

5.8.2) Results

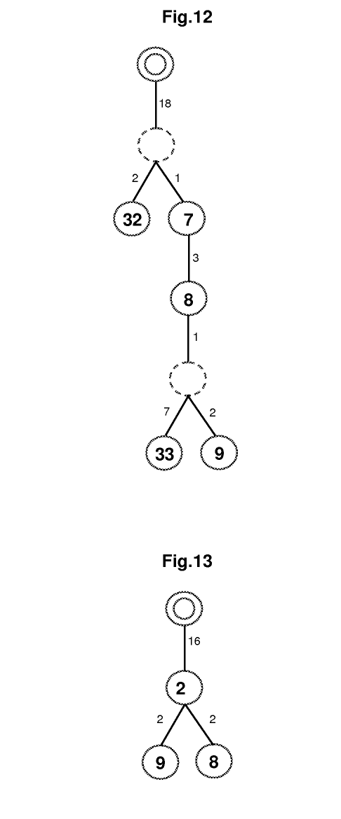

Results are shown in FIG. 12 and FIG. 13.

### Example 6

### Preparation of the Expression System with Sequences from Coronary Plaque Sample and Transformation of Host Cells

Clones or light and heavy chain are then selected for transfection, in particular, clone 8 of the light chain (corresponding to Sequence ID n° 53) and clones 11, 9, 13 and 20 of the heavy chains (corresponding to Sequence ID n° 21, 43, 51 and 37, respectively) of the coronary plaque sample are selected to be transfected into the expression vector for the preparation of the soluble Fab fragments according to the following procedure.

Gene encoding for the light chains selected according to the above Example 6 and corresponding to Sequence ID n° 53 is transferred into the expression vector pRB/expr and following the procedure disclosed by Burioni et al. Hum. Antibodies. 2001; 10(3-4):149-54.

Seq. ID n^{o} 53
GAGCTCACGCAGTCTCCAGCCACCGTGTCTGTGTCTCCAGGGGAAAGAGC
CACCCTCTCCTGCAGGGCCAGTCAGAGTATTAGTTTCCACTTAGCCTGGT
ACCAGCAGAAACCTGGCCAGGCTCCCAGTCTCCTCATCTACGGAACATCC
ACCAGGGCCACTGGTATCCCAGCCAGGTTCAGTGGCAGTGGGTCTGGGAC
AGAGTTCACTCTCACCATCAGCAGCCTGCAGTCTGAAGATTCTGCGGTTT
ATTACTGTCAGCAGTATCATAACTGGCCTCCCCTCACTTTCGGCGGAGGG
ACC

In the expression vector comprising the gene coding for the selected light chain (clone 8 selected from Example 5) is further introduced the gene coding for the heavy chain corresponding to the clone 11 (corresponding to Sequence ID n° 21) following the same procedure disclosed by Burioni et al. Hum Antibodies. 2001; 10(3-4):149-54.

Seq. ID n^{o} 21
CTCGAGTCTGGGGGAGGCTTGGGACAGCCTGGGGGGTCCCTGAGACTCTC
CTGTGCAGCCTCTGGATTTACCTTTAGCAGCTATGCCATGAGCTGGGTCC
GCCAGGCTCCAGGGAAGGGGCTGGAGTGGGTCTCAGCTATTAGTGATAGG
GGGGAGAGCACATACTACGCAGACTCCGTGAAGGGCCGGTTCACCATCTC
CAGGGACAATTCTAAGAACACGCTGTATGTGCAAATGAACAGCCTGAGAG
CCGAGGACACGGCCCTATATTTCTGCGCGAAAGATCAATTTCTATGGTTC
GGGGAGTCAACAGCGGGTGATGCTTTTGATATCTGGGGCCAAGGGACA

The expression vector is introduced into the E. coli XL-1 Blue for the expression of soluble Fabs.

In particular, 10 ml of SB (Super Broth, Becton, Dickinson, N.J.) with ampicillin (100 ng/ml, Sigma-Aldrich, St Louis, Mo.) and tetrayicline (10 ng/ml, Sigma-Aldrich, St Louis, Mo.) is inoculated with a single bacterial colony from a fresh plate and incubated overnight at 37° C. in an orbital shaker

After that, 2.5 ml of this colture is inoculated into 1 liter of SB/amp-tet (the above mixture of SB, ampicillin and tetracyclin) into a 5 liter flask and allowed to grow until an Optical Density (OD₆₀₀) of approximately 1.0. Then IPTG (isopropyl-beta-D-thiogalactopyranoside; Biorad, California) is added up to a final concentration of 1 mM and the bacterial culture are incubated overnight at 30° C. in the orbital shaker. Thus, bacteria are centrifuged at 3000 rpm for 20 minutes at 4° C. and the pellets are resuspended in 10 ml PBS. Subsequently, 50 μl PMSF (from a stock solution of 100 mM) is added in order to inhibit the proteases and bacteria are sonicated three times in ice, 3 minutes for each run. The bacterial culture is centrifuged at 18000 rpm for 45 minutes at 4° C. and the supernatant is filtered carefully with a 0.22 μm diameter membrane (Millipore®). Meanwhile, the column is washed with 10 volumes of PBS and subsequently the filtered supernatant is added slowly to the column. After washing with at least 30 volumes of PBS, Fabs are eluted with 100 mM glycine/HCl pH 2.5. 10 fractions are collected (each one of about 1 ml) and immediately neutralized with Tris 1M pH 9.

Purified Fabs are tested in SDS-PAGE gel in non-reducing conditions showing a single band of approximately 50 kDa.

Fabs are quantified comparing the relative band with at least two different standard concentrations of BSA.

### Example 7

### Preparation of the Expression System with Sequences from Atherosclerotic Plaque Sample and Transformation of Host Cells

The same procedure disclosed in Example 6 is repeated by introducing into the expression vector comprising the gene for the light chain of clone 8 selected according to Example 5, the sequence coding for the heavy chain of clone 9 (corresponding to Sequence ID n° 43).

Seq ID n^{o} 43
CTCGAGTCTGGGGGAGGCTTGGTCAAGCCTGGAGGGTCCCTGAGGCTCTC
CTGTGCAGCCTCTGGATTCACCTTCAGTGACTACTACATGAGTTGGATCC
GCCAGGCTCCAGGGAAGGGGCTGGAATTTATATCATACATTAGTAGTGGT
GGTGACACCATACACCACGCAGACTCTGTGAAGGGCCGATTCACCATCTC
CAGGGACAACGCCAAGAAGTCACTGTATCTCCAAATGAACAGCCTGAGAG
TCGAGGACACGGCCGTATATTACTGTGCGTGCCGTGGGGTCTGGGGCCAG
GGAACC

### Example 8

### Preparation of the Expression System with Sequences from Atherosclerotic Plaque Sample and Transformation of Host Cells

The same procedure disclosed in Example 6 is repeated by introducing into the expression vector comprising the gene for the light chain of clone 8 selected according to Example 5, the sequence coding for the heavy chain of clone 13 (corresponding to Sequence ID n° 51).

Seq. ID n^{o} 51
CTCGAGTCGGGCCCAGGACTGGTGAAGCCTTCACAGACCCTGTCCCTCAC
CTGCACTGTCTCTGGTGGCTCCATCAGCAGTGGTTACTACTGGACCTGGA
TCCGCCAGTACCCAGGGAGGGGCCTGGAGTGGATTGGATACATCTCTTAC
AGGGGGAGCACCTACTACAACCCGTCCCTCAAGAGTCGAGTTACAATATC
ACTAGACACGTCTAAGAACCAGTTTTTCTTGAACCTGACCTCTGTGACTG
CCGCGGACACGGCCGTGTATTTCTGTGCGAGAGATCGGAGTAGAGCAACA
TCTGGTATTCTTGACTACTGGGGCCAGGGAACC

### Example 9

### Preparation of the Expression System with Sequences from Atherosclerotic Plaque Sample and Transformation of Host Cells

The same procedure disclosed in Example 6 is repeated by introducing into the expression vector comprising the gene for the light chain of clone 8 selected according to Example 5, the sequence coding for the heavy chain of clone 20 (corresponding to Sequence ID n° 37).

Seq. ID n^{o} 37
CTCGAGTCGGGGGGAGGCTTCGTACAGCCTGGGGGGTCTCTGAGACTCTC
CTGTGCAGCCTCTGGATTCACCTTCAGGGACTATGCCATGGGCTGGGTCC
GCCAGGCTCCAGGGAAGGGGCCGGAGTGGGTCTCAATTATTAGTGCTAGT
GGTGGTTCCATATACTACGCAGACTCCGTGAAGGGCCGATTCACCATCTC
CAGAGACAACGCCAAGAACACACTGTATCTGCAAATGAACAGTCTCAGAG
CCGACGACACGGCTGTATACTACTGTGCAAGACAGACCAGCAGCAGATGG
TATGATTGGTTCGACCCCTGGGGCCAGGGAACC

### Example 10

### Immunohystologic Assay

A fresh sample of plaque is frozen in liquid nitrogen and sectioned using a cryostat. Sections 5 μm thick are fixed with ice-cold acetone and blocked with a serum blocking solution (2% serum, 1% BSA, 0.1% Triton X-100, 0.05% Tween 20) for 1 hour at room temperature. The fixed sections are probed with the Fabs produced and identified according to the present invention, at an appropriate dilution, and incubated for 2 hours at room temperature. Sections are washed five times with PBS and an appropriate dilution of a FITC (fluorescein isothiocyanate)-conjugated secondary anti-human Fab (Sigma-Aldrich, St Louis, Mo.) is added. After 30 minutes at room temperature, sections are washed again and the complex ligand/antibody thus formed is detected with a fluorescence microscope.

### Example 11

### Antibody Screening of Phage Library

Panning of the random phage-displayed peptide library expressing dodecapeptides at the N-terminus of cpIII coat protein of the filamentous phage M13 (Ph.D.12™ Phage Display Peptide Library Kit, Catalog #E8110S, New England Biolabs, Beverly, Mass.) is performed according to the manufacturer's instructions using Fab-coated high-binding 96-well ELISA plates (Costar 96w polystyrene ½ area flat bottom HI-binding flat bottom, cat #3690).

In order to remove phages binding to antibody conserved regions, a negative selection is performed from the second round of panning by combining the amplified phages with 25 μg of a pool of human standard IgG (Endobulin, A.T.0 J06BA02, Baxter S.p.A.) for 1 hour at 37° C.

Four rounds of selection are performed as described above, panning the amplified phage on Fabs produced and identified according to the present invention and the same pool of standard IgG used for the negative selection.

### Example 12

### Peptide Screening and DNA Sequence Analysis

All the phages obtained as from Example 11 are used to infect E. coli strain ER2537 and randomly picked single plaques are screened in enzyme-linked immunoassay on Fabs produced and identified according to the present invention and the pool of standard IgG.

Antigen-coated plates (Costar 96w polystyrene ½ area flat bottom HI-binding flat bottom, cat #3690) are ished and blocked with a solution of PBS/BSA 1% for 1 hour at 37° C.; 50 μl of 10⁸ phages per milliliter are added and incubated for 2 hours at 37° C.

Plates are washed 10 times with PBS (0.1% Tween-20; Sigma-Aldrich, St Louis, Mo.); afterward, 50 μl of a 1:3000 dilution in PBS of a HRP-conjugated anti-M13 antibody (GE Healthcare 27-9411-01) is added.

After 2 hours at 37° C. plates are washed P with PBS (0.5% Tween-20; Sigma-Aldrich, St Louis, Mo.), specific bound phages are detected by adding 100 μl of substrate (Sigma-Aldrich, St Louis, Mo.) and plates are read for an Optical Density of 450 nm after 30 minutes at room temperature.

Positive clones showing an OD_{450 nm} value >1 on Fabs of the present invention and OD_{450 nm} value <0.3 on pool of IgG are scored as positives and evaluated by sequence analysis using the software Pepitope http://pepitope.tau.ac.il/index.html. From peptide sequence analisys conserved aminoacidic positions are identified and four peptides are selected on the basis of the amount of consensus residues present in their sequences.

Four peptides have been identified, and the related sequences corresponding to Sequence ID from 391 to 394.

### Example 13

### Enzyme-Linked ImmunoSorbent Assay

Hep-2 (ATCC CCL-23) cells are grown in E-Mem (Invitrogen 0820234DJ) supplemented with Antibiotic/Antimycotic Solution (Invitrogen, Antibiotic/Antimycotic Solution, liquid 15240-062) and 10% FBS. Cells are regularly split 1:10 every 5 days. Five million cells are washed in PBS and lysed by using RIPA buffer (50 mM Tris HCL ph8+150 mM NaCl+1% NP-40+0.5% NA deossicolate+0.1% SDS).

Elisa plates (Costar 96w polystyrene ½ area flat bottom HI-binding flat bottom, cat #3690) are coated with serial dilution of of Hep-2 Lysate (1000 ng, 200 ng, 40 ng and 8 ng in PBS) overnight at 4°. After blocking with PBS+BSA3% for 2 hours at 37° C., serial dilutions of Fab 24 (20 μg/ml, 10 μg/ml, 5 μg/ml, 2.5 μg/ml, are incubated with the coated antigens for 1 hour at 37° C. After washing with PBS+Tween20 0.1% (SIGMA cod. PL379), plates are incubated with anti human IgG peroxidase (SIGMA cod. A2290) for 30 minutes at 37° C. After washing with PBS+Tween 0.1%, TMB substrate was added to the wells (PIERCE TMB substrate kit for peroxidase cod. SK 4400). ELISA plates are analysed with a spectrophotometer at 450 nm.

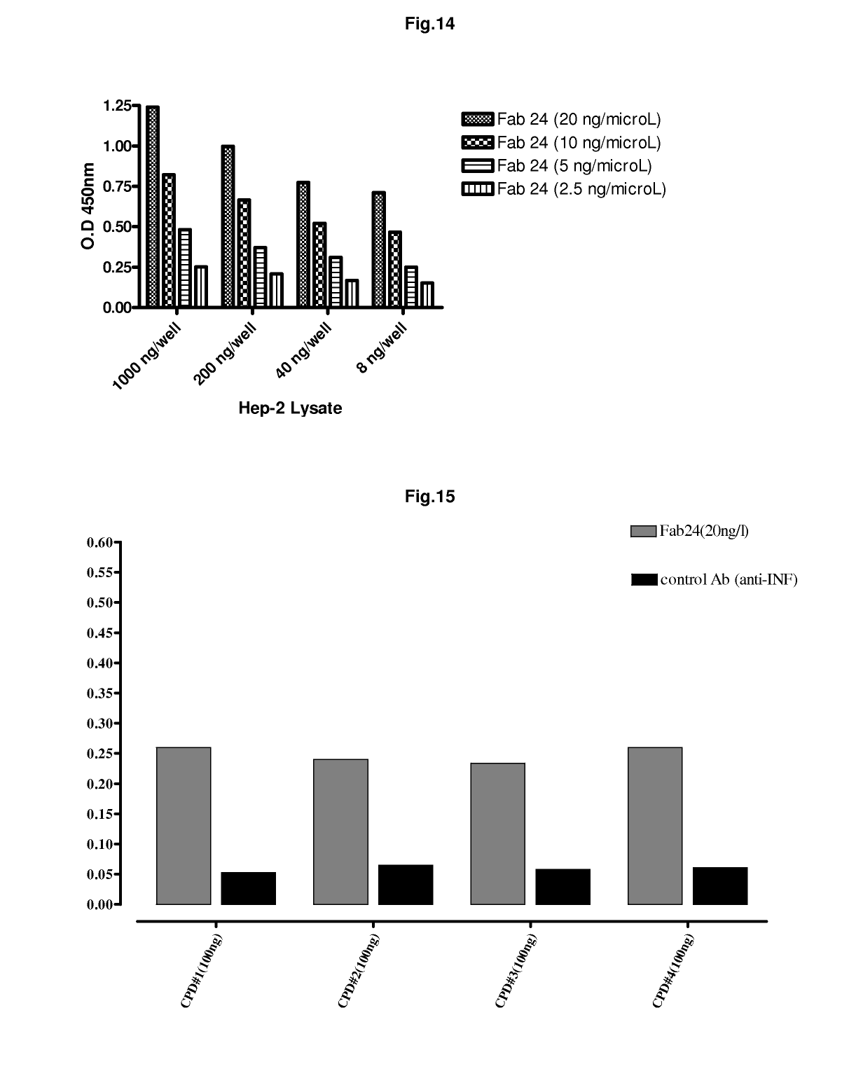

Results are shown in FIG. 14.

### Example 14

### Synthesis of the Peptides

14.1) General

Abbreviations for Chemical Reagents, Chemical Structure Moieties and Techniques: AA—amino acid, AcOH—Acetic acid, ACN—Acetonitrile, API-ES—Atmospheric pressure ionization electrospray, Btn—Biotin, Boc—tert-Butyloxycarbonyl, DCM—Dichloromethane, DIC—N,N-Diisopropylcarbodiimide, DIEA—N,N-Diisopropylethylamine, DMF—N,N-Dimethylformamide, Et₂O—Diethyl ether, Fmoc-9-Fluorenylmethoxycarbonyl, Adoa-8-Amino-3,6-dioxaoctanoic acid, HFIP-1,1,1,3,3,3-hexafluoro-2-propanol, HOBt—N-Hydroxybenzotriazole, MeOH—Methanol, Neg. ion—Negative ion, NHS—N-Hydroxysuccinimide, NMP—N-Methylpyrrolidone, Pip—Piperidine, Pos. ion—Positive ion, HBTU-O—(Benzotriazol-1-yl)-N,N,N′,N′-tetramethyluronium hexafluorophosphate, PyBOP—Benzotriazole-1-yl-oxy-tris-pyrrolidino-phosphonium hexfluorophosphate, t_{R}—Retention time (minutes), Reagent B (88:5:5:2—TFA:H₂O:phenol:TIPS—v/v/wt/v), Su—Succinimidyl, TFA—Trifluoroacetic Acid, TIPS—Triisopropylsilane, H₂O—Water.

Names, structures and abbreviations used for amines and unnatural amino acids used in the synthesis of various peptides are given in Table V.

Solvents for reactions, chromatographic purification and HPLC analyses are E. Merck Omni grade solvents from VWR Corporation (West Chester, Pa.). NMP and DMF are purchased from Pharmco Products Inc. (Brookfield, Conn.), and are peptide synthesis grade or low water/amine-free Biotech grade quality. Piperidine (sequencing grade, redistilled 99+%) and TFA (spectrophotometric grade or sequencing grade) are purchased from Sigma-Aldrich Corporation (Milwaukee, Wis.) or from the Fluka Chemical Division of Sigma-Alrich Corporation. Phenol (99%), DIEA, DIC and TIPS are purchased from Sigma-Aldrich Corporation. Fmoc-protected amino acids, PyBop, and HOBt used are purchased from Nova-Biochem (San Diego, Calif., USA), Advanced ChemTech (Louisville, Ky., USA), Chem-Impex International (Wood Dale Ill., USA), and Multiple Peptide Systems (San Diego, Calif., USA). Fmoc-Adoa and Btn-Adoa-Adoa-OH are obtained from NeoMPS Corp (San Diego, Calif.).

Analytical HPLC data are generally obtained using a Shimadzu LC-10AT VP dual pump gradient system employing either Waters X-Terra® MS-C18 (5.0μ, 50×4.6 mm; 120 Å pore size) or Waters Sunfire™ OBD-C8 (4.6×50 mm 3.5μ, 120 Å pore size) columns and gradient or isocratic elution systems using H₂O (0.1% TFA) as eluent A and ACN (0.1% TFA) as eluent B. Detection of compounds is accomplished using UV at 220 and/or 230 nm.

Preparative HPLC is conducted on a Shimadzu LC-8A dual pump gradient system equipped with a SPD-10AV UV detector fitted with a preparative flow cell. Generally the solution containing the crude peptide is loaded onto a reversed phase Waters Sunfire™ OBD C8 (50×250 mm; particle size: 10.0μ, 120 Å pore size) column, using a third pump attached to the preparative Shimadzu LC-8A dual pump gradient system. After the solution of the crude product mixture is applied to the preparative HPLC column the reaction solvents and solvents employed as diluents, such as DMF or DMSO, are eluted from the column at low organic phase composition. Then the desired product is eluted using a gradient elution of eluent B into eluent A. Product-containing fractions are combined based on their purity as determined by analytical HPLC and mass spectral analysis. The combined fractions are freeze-dried to provide the desired product.

Mass spectral data are obtained in-house on an Agilent LC-MSD 1100 Mass Spectrometer. For the purposes of fraction selection and characterization of the products, mass spectral values are usually obtained using API-ES in positive ion mode. Generally the molecular weight of the target peptides is ˜2000; the mass spectra usually exhibited strong doubly or triply positively charged ion mass values rather than weak [M+H]⁺. These are generally employed for selection of fractions for collection and combination to obtain the pure peptide from HPLC purification.

14.2) General Methods for Solid Phase Peptide Synthesis (SPPS)

14.2.1) The linear peptides are synthesized by an established automated protocol on a Rainin PTI Symphony® Peptide Synthesizer (twelve peptide sequences/synthesis) using Fmoc-Pal-Peg-PS resin (0.2 mmol/g) and/or suitably preloaded resins, Fmoc-protected amino acids and PyBop-mediated ester activation in DMF. The rest of the peptide sequence is loaded on the Fmoc-Pal-Peg-PS and/or other resins in stepwise fashion by SPPS methods typically on a 0.2 mmol scale. The amino acid coupling is carried out with a 4-fold excess each of amino acid activated by PyBop-DIEA reagent in DMF. Biotin is coupled to N-terminus of the peptide with a four-fold excess of Btn-NHS ester.

14.2.2) When preloaded diamines on trityl resins are used, after final acetylation the fully protected peptide chain is cleaved from the resin with 8:1:1—DCM: AcOH: HFIP and after evaporation of the volatiles, the final Btn-Adoa-Adoa is coupled to the amine at the C-terminus in solution. The crude fully protected peptide is treated with 1.0 equivalent of pre-formed Btn-Adoa-Adoa-NHS ester in solution for 16 h at RT (total volume 5.0 mL/g of the crude weight).

In a typical coupling process for a given amino acid, 6.0 mL of DMF solution containing 0.8 mmol of an amino acid followed by PyBOP (0.8 mmol, DMF solution, 1.25 mL) and DIEA (0.8 mmol, DMF solution, 1.25 mL) are added in succession by an automated protocol to a reaction vessel (RV) containing the resin (0.2 mmol) and the resin is agitated by recurrent nitrogen bubbling. After 1 hour the resin is washed thoroughly with DMF (4.5 mL, 6×) and the cleavage of the Fmoc-group is performed with 25% Pip in DMF (4.5 mL) for 10 minutes followed by a second treatment with the same reagent for 10 minutes to ensure complete deprotection. Again the resin is thoroughly washed with DMF (5 mL/g, 6×); a DCM (10 mL/g) wash is performed in between DMF washes to guarantee that the resin is freed of residual Pip. After completion of the peptide synthesis, the resin bearing the fully protected peptide was cleaved with, Reagent B (10.0 mL/g of resin or 10.0 mL/g of crude protected peptide) for 4 hours. The volatiles are removed under vacuum to provide a paste. The paste thus obtained is triturated with Et₂O to provide a solid which was pelleted by centrifugation followed by 3 more cycles of Et₂O washing and pelleting. The resulting solid is dried under vacuum to provide the crude peptide. A 200 μmol scale synthesis of a peptide of MW˜2000 gave 200-300 mg (90-110% of theory) of the crude peptide. The greater than theoretical yield is most likely due to moisture and residual solvents.

14.3) Purification of Peptides—General Procedure

A 200 μmol scale synthesis of a peptide of MW˜2000 on the ‘Symphony’ instrument provided˜200-300 mg of crude peptide from each reaction vessel (RV). The crude peptide (˜200-500 mg) is purified in one run by reversed phase HPLC. The crude peptide (˜200 mg) dissolved in ACN (10 mL) is diluted to a final volume of 50 mL with H₂O and the solution is filtered. The filtered solution is loaded onto a preparative HPLC column (Waters, Sunfire™ Prep C8, 50×250 mm 10μ, 120 Å) which had been pre-equilibrated with 10% ACN in H₂O (0.1% TFA). During the application of the solution to the column the flow of the equilibrating eluent from the preparative HPLC system is stopped. After the solution is applied to the column, the flow of equilibrating eluent from the gradient HPLC system is reinitiated and the composition of the eluent is then ramped to 20% ACN-H₂O (0.1%TFA) over 10.0 minutes after which a linear gradient at a rate of 0.5%/min of ACN (0.1% TFA) into H₂O (0.1% TFA) is initiated and maintained for 60 minutes. Fractions (15 mL) are collected using UV at 220 nm as an indicator of product elution. The collected fractions are analyzed on an analytical reversed phase C8 column (Waters Sunfire™ OBD-C8, 4.6×50 mm, 5μ, 120 Å) and product-containing fractions of >95% purity are combined and freeze-dried to afford the corresponding peptide. After isolation, the peptides are analyzed by HPLC and mass spectrometry to confirm identity and purity. Data for the peptides are provided in Table VI (Sequence, Resin

Loading and Yield), Table VII (HPLC and Mass Spectral Analysis) and Table VIII (Peptide Structures).

### Example 15

### Enzyme-Linked ImmunoSorbent Assay

Elisa plates (Costar 96w polystyrene ½ area flat bottom HI-binding flat bottom, cat #3690) are coated with 100 ng of peptides resuspended in PBS overnight at 4° C. After blocking with PBS+BSA3% for 2 hours at 37° C., Fab 24 (20 μg/ml) are incubated with the coated antigens for 1 hour at 37° C. After washing with PBS+Tween20 0.1% (SIGMA cod: PL379), plates are incubated with anti human IgG peroxidase (SIGMA cod: A2290) for 30 minutes at 37° C. After washing with PBS+Tween 0.1%, TMB substrate is added to the wells (PIERCE TMB substrate kit for peroxidase cod: SK 4400). ELISA plates are analysed with a spectrophotometer at 450 nm.

Results are shown in FIG. 15.

TABLE V
Abbreviations and Structures

| Abbreviation | Structure |
|---|---|
| Adoa | 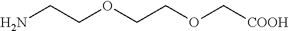 |
| Btn | 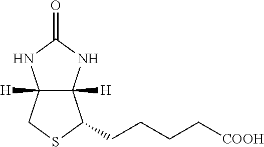 |
| EDA | H₂N—CH₂—CH₂—NH₂ |

TABLE VI
Peptide Sequence, Resin Loading and Yield

| | | Resin used, Loading, | Yield in |
|---|---|---|---|
| CPD# | Sequence | mmol/g, g, mmol | mg (%) |
| 1 | Ac-TMGFTAPRFPHY-NH₂ | Fmoc-PAL-PEG-PS, | 167.0 |
| | | 0.2 mmol/g, 1.2 g, 0.24 | (46%) |
| | | mmol | |
| 2 | Ac-MQSPFTPHFAER-NH₂ | Fmoc-PAL-PEG-PS, | 137.0 |
| | | 0.2 mmol/g, 1.2 g, 0.24 | (38%) |
| | | mmol | |
| 3 | Ac-MQSPIVLPLSLS-NH₂ | Fmoc-PAL-PEG-PS, | 131.0 |
| | | 0.2 mmol/g, 1.2 g, 0.24 | (41%) |
| | | mmol | |
| 4 | Ac-HHEPGAWLPLSP-NH₂ | Fmoc-PAL-PEG-PS, | 209.0 |
| | | 0.2 mmol/g, 1.2 g, 0.24 | (62%) |
| | | mmol | |
| 5 | Btn-Adoa-Adoa- | Fmoc-PAL-PEG-PS, | 70.0 |
| | TMGFTAPRFPHY-NH₂ | 0.2 mmol/g, 1.0 g, 0.2 | (18%) |
| | | mmol | |
| 6 | Btn-Adoa-Adoa-MQSPIVLPLSLS- | Fmoc-PAL-PEG-PS, | 140.0 |
| | NH₂ | 0.2 mmol/g, 1.0 g, 0.2 | (38%) |
| | | mmol | |
| 7 | Btn-Adoa-Adoa- | Fmoc-PAL-PEG-PS, | 205.0 |
| | HHEPGAWLPLSP- NH₂ | 0.2 mmol/g, 1.0 g, 0.2 | (55%) |
| | | mmol | |
| 8 | Btn-Adoa-Adoa- | Fmoc-PAL-PEG-PS, | 135.0 |
| | MQSPFTPHFAER-NH₂ | 0.2 mmol/g, 1.0 g, 0.2 | (34%) |
| | | mmol | |
| 9 | Ac-TMGFTAPRFPHY-DAE-Adoa- | 1,2-Diaminoethane trityl | 65.0 |
| | Adoa-Btn | resin, 0.9 mmol/g, | (16%) |
| | | 0.222 g, 0.2 mmol | |
| 10 | Ac-MQSPFTPHFAER-DAE-Adoa- | 1,2-Diaminoethane trityl | 30.0 |
| | Adoa-Btn | resin, 0.9 mmol/g, | (7%) |
| | | 0.222 g, 0.2 mmol | |
| 11 | Ac-MQSPIVLPLSLS-DAE-Adoa- | 1,2-Diaminoethane trityl | 60.0 |
| | Adoa-Btn | resin, 0.9 mmol/g, | (15.7%) |
| | | 0.222 g, 0.2 mmol | |
| 12 | Ac-HHEPGAWLPLSP-DAE-Adoa- | 1,2-Diaminoethane trityl | 25.0 |
| | Adoa-Btn | resin, 0.9 mmol/g, | (7%) |
| | | 0.222 g, 0.2 mmol | |

TABLE VII
HPLC and Mass Spectral Analysis of Peptides

| Cpd # | RT, Column & Conditions | MS |
|---|---|---|
| 1 | Ret. time: 7.38 min; Assay: >95% (area %); | [M + H]: |
| | Column: Waters X-Terra MS C-18 RP, | 1465.6, |
| | 50.0 mm × 4.6 mm i.d.; Particle size: 5.0 | [M + 2H]/2: |
| | microns; Eluents: A: Water (0.1% TFA), B: | 733.4 |
| | acetonitrile (0.1% TFA); Elution: Initial | |
| | condition: 10.0% B, linear gradient 10-40% | |
| | B over 15.0 min; Flow rate: 3.0 mL/min; | |
| | Detection: UV @ 220 nm. | |
| 2 | Ret. time: 6.48 min; Assay: >95% (area %); | [M + H]: |
| | Column: Waters X-Terra MS-C18 RP, | 1489.6; |
| | 50.0 mm × 4.6 mm i.d.; Particle size: 5.0 | [M + Na + H]: |
| | microns; Eluents: A: Water (0.1% TFA), B: | 755.0; |
| | acetonitrile (0.1% TFA); Elution: Initial | [M + 2H]/2: |
| | condition: 10.0% B, linear gradient 10-40% | 744.8 |
| | B over 15.0 min; Flow rate: 3.0 mL/min; | |
| | Detection: UV @ 220 nm. | |
| 3 | Ret. time: 10.14 min; Assay: >95% (area | [M + K]: |
| | %); Column: Waters X-Terra MS-C18 RP, | 1364.6; |
| | 50.0 mm × 4.6 mm i.d.; Particle size: 5.0 | [M + Na]: |
| | microns; Eluents: A: Water (0.1% TFA), B: | 1348.6 |
| | acetonitrile (0.1% TFA); Elution: Initial | |
| | condition: 10.0% B, linear gradient 10-40% | |
| | B over 15.0 min; Flow rate: 3.0 mL/min; | |
| | Detection: UV @ 220 nm. | |
| 4 | Ret. time: 6.86 min; Assay: >95% (area %); | [M + H]: |
| | Column: Waters X-Terra MS C-18 RP, | 1382.6; |
| | 50.0 mm × 4.6 mm i.d.; Particle size: 5.0 | [M + Na]: |
| | microns; Eluents: A: Water (0.1% TFA), B: | 1403.6 |
| | acetonitrile (0.1% TFA); Elution: Initial | |
| | condition: 10.0% B, linear gradient 10-40% | |
| | B over 15.0 min; Flow rate: 3.0 mL/min; | |
| | Detection: UV @ 220 nm. | |
| 5 | Ret. time: 5.63 min; Assay: >95% (area %); | [M + H]: |
| | Column: Waters Sunfire ™ C-8 RP, | 1940.6; |
| | 50.0 mm × 4.6 mm i.d.; Particle size: 3.5 | [M + 2H]/2: |
| | microns; Eluents: A: Water (0.1% TFA), B: | 970.8 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm. | |
| 6 | Ret. time: 8.53 min; Assay: >95% (area %); | [M + Na]: |
| | Column: Waters Sunfire ™ C-8 RP, | 1823.8; |
| | 50.0 mm × 4.6 mm i.d.; Particle size: 3.5 | [M + H]: |
| | microns; Eluents: A: Water (0.1% TFA), B: | 1800.8; |
| | acetonitrile (0.1% TFA); Elution: Initial | [M + 2Na]/2; |
| | condition: 15.0% B, linear gradient 15-45% | 922.5; |
| | B over 15.0 min; Flow rate: 3.0 mL/min; | [M + 2H]/2: |
| | Detection: UV @ 220 nm | 900.5 |
| 7 | Ret. time: 5.57 min; Assay: >95% (area %); | [M + H]: |
| | Column: Waters Sunfire ™ C-8 RP, | 1855.5; |
| | 50.0 mm × 4.6 mm i.d.; Particle size: 3.5 | [M + 2H]/2: |
| | microns; Eluents: A: Water (0.1% TFA), B: | 928.5 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |
| 8 | Ret. time: 5.29 min; Assay: >95% (area %); | [M + H]: |
| | Column: Waters Sunfire ™ C-8 RP, | 1964.5; |
| | 50.0 mm × 4.6 mm i.d.; Particle size: 3.5 | [M + 2H]/2: |
| | microns; Eluents: A: Water (0.1% TFA), B: | 982.0 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |
| 9 | Ret. time: 5.73 min; Assay: >95% (area %); | [M + H]: |
| | Column: Waters Sunfire ™ C-8 RP, | 2025.5; |
| | 50.0 mm × 4.6 mm i.d.; Particle size: 3.5 | [M + 2H]/2: |
| | microns; Eluents: A: Water (0.1% TFA), B: | 1013.3 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |
| 10 | Ret. time: 5.29 min; Assay: >90% (area %); | [M + H]: |
| | Column: Waters Sunfire ™ C-8 RP, | 2047.8; |
| | 50.0 mm × 4.6 mm i.d.; Particle size: 3.5 | [M + 2H]/2: |
| | microns; Eluents: A: Water (0.1% TFA), B: | 1024.7 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |
| 11 | Ret. time: 8.41 min; Assay: >90% (area %); | [M + H]: |
| | Column: Waters Sunfire ™ C-8 RP, | 1906.8; |
| | 50.0 mm × 4.6 mm i.d.; Particle size: 3.5 | [M + 2H]/2: |
| | microns; Eluents: A: Water (0.1% TFA), B: | 965.0 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |
| 12 | Ret. time: 5.73 min; Assay: >95% (area %); | [M + H]: |
| | Column: Waters Sunfire ™ C-8 RP, | 1940.8; |
| | 50.0 mm × 4.6 mm i.d.; Particle size: 3.5 | [M + 2H]/2: |
| | microns; Eluents: A: Water (0.1% TFA), B: | 971.0 |
| | acetonitrile (0.1% TFA); Elution: Initial | |
| | condition: 15.0% B, linear gradient 15-45% | |
| | B over 15.0 min; Flow rate: 3.0 mL/min; | |
| | Detection: UV @ 220 nm | |

TABLE VIII
Structures of Peptides
Compound 1
Ac-TMGFTAPRFPHY-NH₂

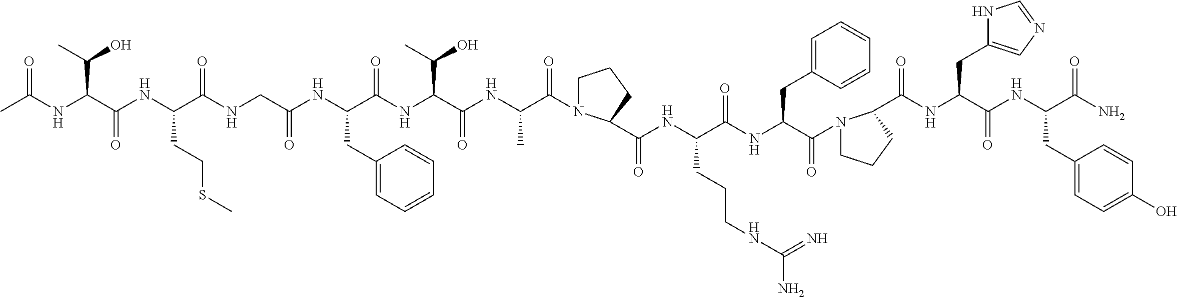

Compound 2
Ac-MQSPFTPHFAER-NH₂

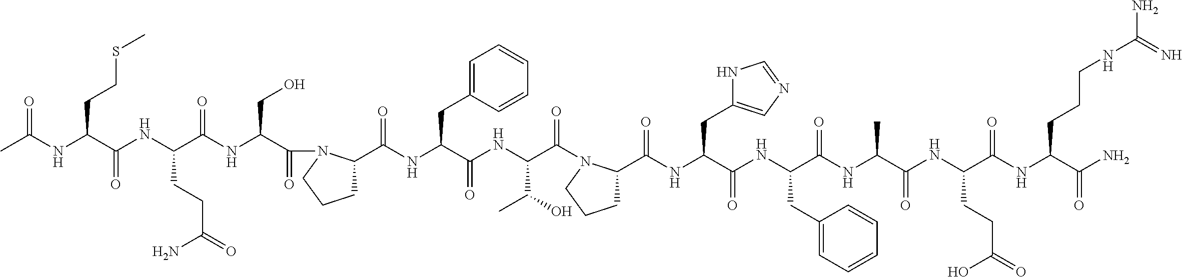

Compound 3
Ac-MQSPIVLPLSLS-NH₂

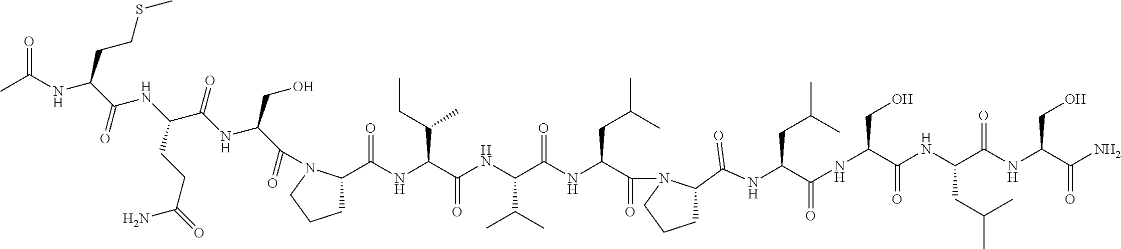

Compound 4
Ac-HHEPGAWLPLSP-NH₂

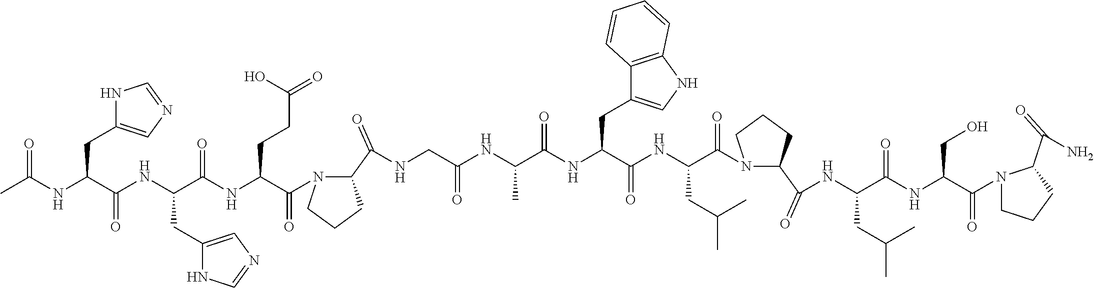

Compound 5
Btn-Adoa-Adoa-TMGFTAPRFPHY-NH₂

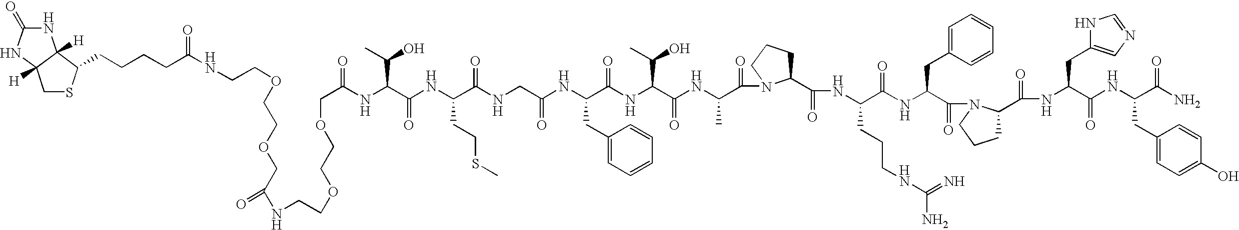

Compound 6
Btn-Adoa-Adoa-MQSPIVLPLSLS-NH₂

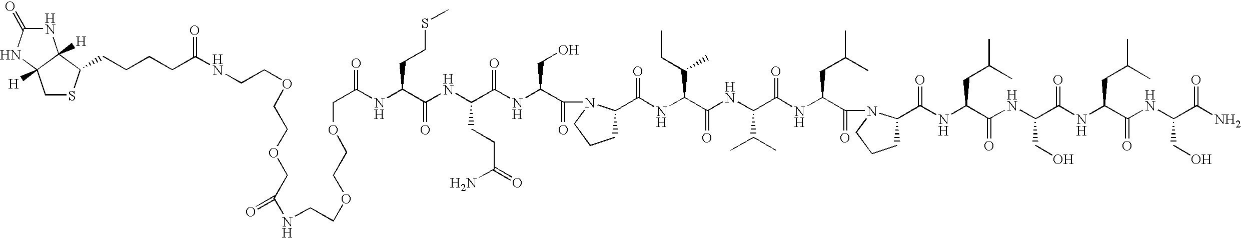

Compound 7
Btn-Adoa-Adoa-HHEPGAWLPLSP-NH₂

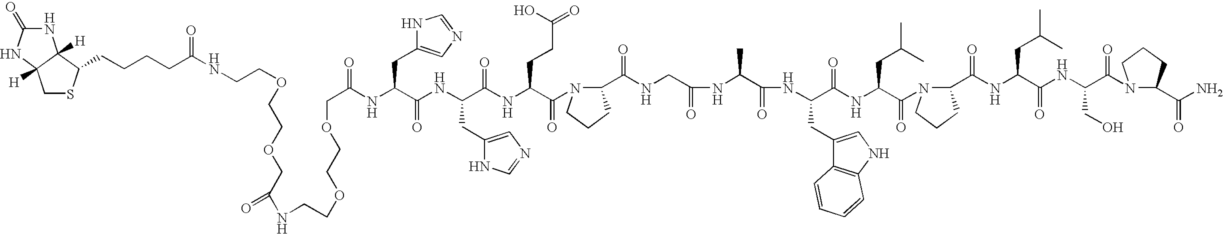

Compound 8
Btn-Adoa-Adoa-MQSPFTPHFAER-NH₂

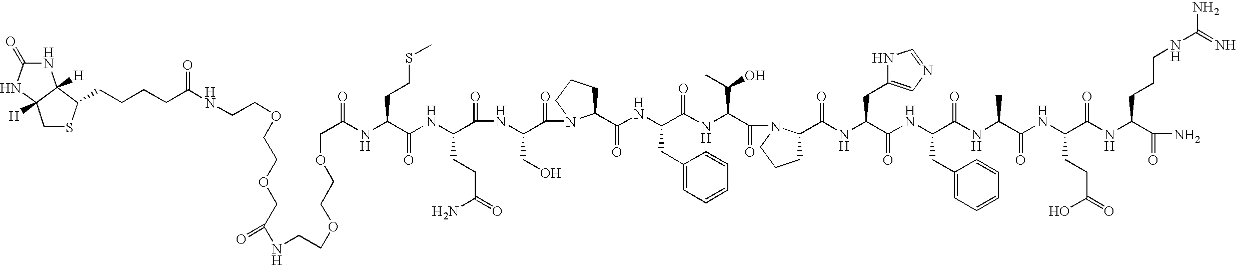

Compound 9
Ac-TMGFTAPRFPHY-DAE-Adoa-Adoa-Btn

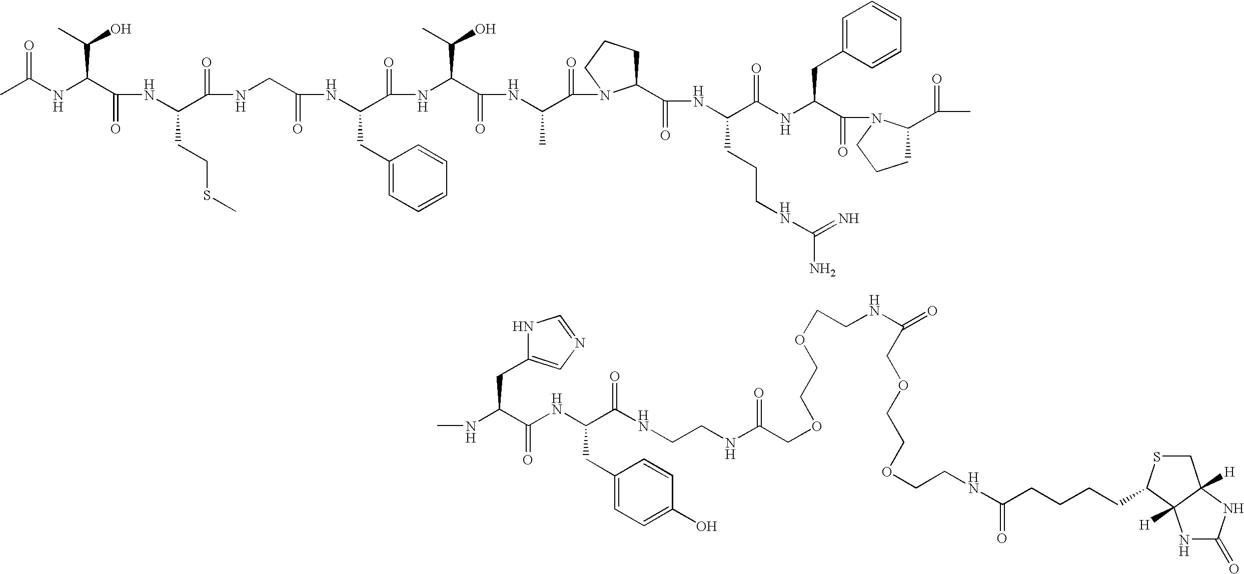

Compound 10
Ac-MQSPFTPHFAER-DAE-Adoa-Adoa-Btn

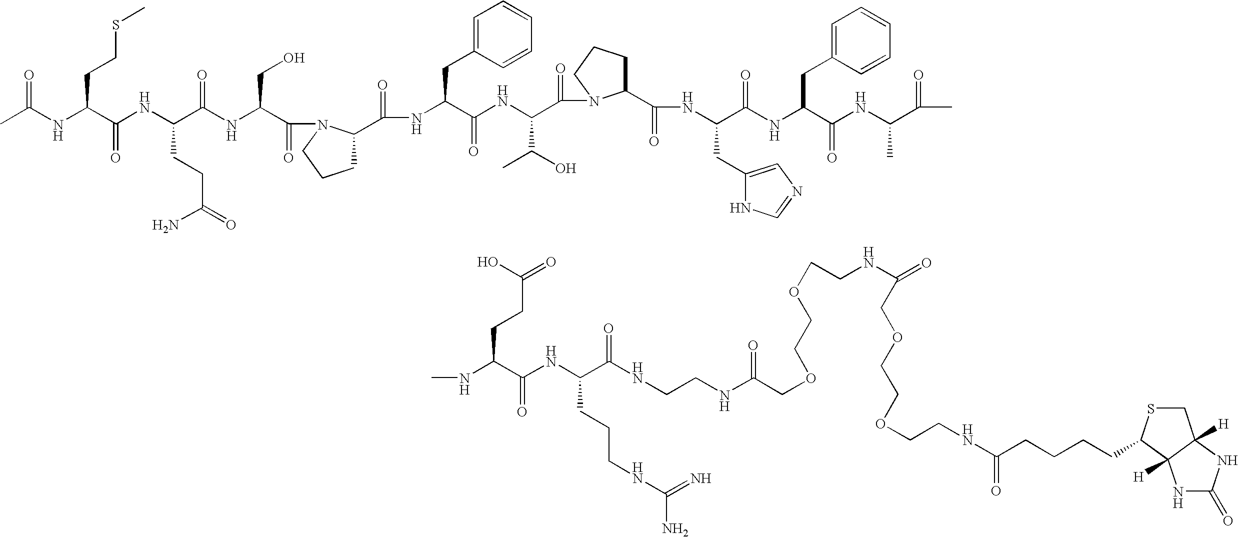

Compound 11
Ac-MQSPIVLPLSLS-DAE-Adoa-Adoa-Btn

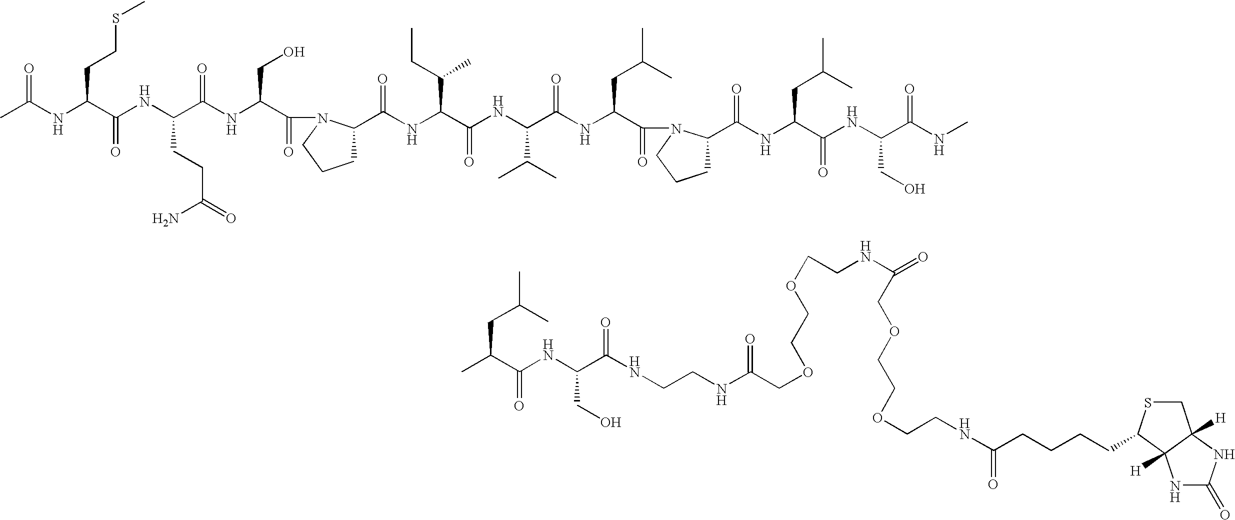

Compound 12
Ac-HHEPGAWLPLSP-DAE-Adoa-Adoa-Btn

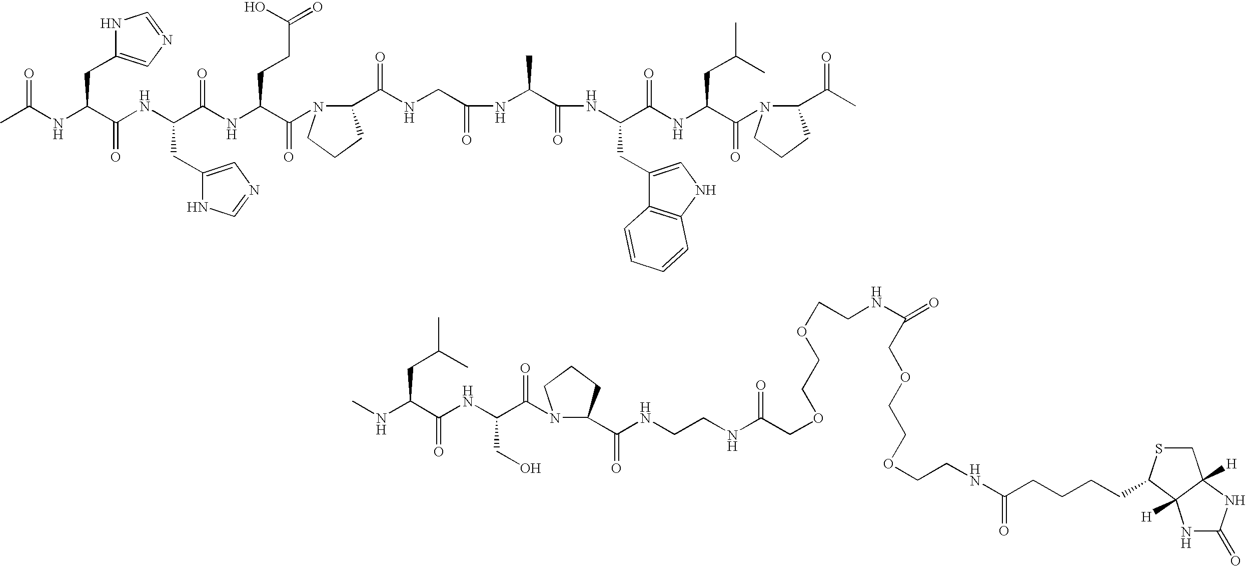

1. An amino acid sequence comprising any one of the even-numbered Sequence ID NOS: 2 to 390 and 396 to 454 or any homologous sequence bearing conservative substitutions, encoded by a polynucleotide isolated from a biopsy of an active coronary plaque.

2. An antibody comprising one or more of the amino acid sequences according to claim 1 and showing binding properties to at least one of a consensus peptide selected from the group consisting of: SEQ ID NOS 391 to 394.

3. The isolated amino acid sequence according to claim 1 selected from the group consisting of: SEQ ID NO:22, SEQ ID NO:38, SEQ ID NO:44, SEQ ID NO:52 and SEQ ID NO:54.

4. The amino acid sequence of claim 1 having a germline homology of at least 95% and even more preferably of at least 97%; or any fragment thereof.

5-6. (canceled)

7. A polynucleotidic molecule isolated from a biopsy of an active coronary plaque comprising: any one of the odd-numbered SEQ ID NOS: 1 to 389 and 395 to 453, encoding for the amino acid sequence of claim 1.

8. (canceled)

9. An expression vector comprising one or more of the isolated polynucleotide molecules of claim 7.

10. The expression vector of claim 9 selected from the group comprising plasmids, cosmids, YACs, viral particles or phages.

11. An expression system comprising one or more expression vectors according to claim 9.

12. An isolated recombinant host cell comprising the expression system of claim 11.

13. The isolated recombinant host cell of claim 12 selected from the group consisting of: prokaryotic recombinant isolated cells; yeast recombinant cells; human recombinant isolated cells; plant isolated recombinant cells; and insect recombinant isolated cells.

14. A process for the preparation of a recombinant antibody comprising any one of the amino acid sequences of claim 1 or any homologous sequence bearing conservative substitutions- or any fragment thereof—including the steps of:
a) preparing an expression system in an isolated host cell comprising any one of the polynucleotidic molecules selected from the group consisting of: any one of the odd-numbered SEQ ID NO: 1 to 389 and 395 to 453, isolated from a biopsy of an active coronary plaque;
b) culturing said host cell under suitable growth conditions; and
c) recovering and/or purifying the antibody or any fragments thereof thus produced.

15. The process of claim 14 further comprising a step d) of testing for any binding activity to an amino acid consensus sequence selected from the group consisting of: SEQ ID NOS: 391 to 394.

16. The process of claim 14 wherein said isolated host cell is selected from the group consisting of: E. coli, B. subtilis, S. Cerevisiae and Chinese hamster ovary (CHO).

17. The process according to claim 14 wherein said antibody is an IgG or any fragment thereof.

18. The process according to claim 14 wherein said fragment is a Fab fragment.

19-23. (canceled)

24. A recombinant isolated antibody or any fragment thereof obtained according to the process of claim 14.

25. A composition comprising the recombinant antibody or any fragment thereof according to claim 2 and, optionally, a moiety.

26. The composition of claim 25 wherein said moiety is a therapeutic moiety selected from the group consisting of: radionuclides, drugs and prodrugs, hormones, hormone antagonists, receptor antagonists, enzymes or proenzymes activated by another agent, autocrines and cytokines, antimicrobial agents and toxins.

27. The composition of claim 25 wherein said moiety is a diagnostic moiety.

28. The composition of claim 26 for the treatment of the acute coronary syndrome (ACS).

29-34. (canceled)

35. The composition of claim 27 for the diagnosis of the acute coronary syndrome (ACS).

36-41. (canceled)

42. A method for identifying a ligand which binds to the antibody or any fragment thereof according to claim 2 comprising the steps of:
a) binding said antibody onto a solid phase;
b) removing unbound material by one or more washing steps;
c) contacting a candidate ligand with the solid phase prepared in step a) and allowing incubation of the candidate ligand and the solid phase for a suitable period of time;
d) removing unbound material by one or more washing steps;
e) adding a secondary antibody specific for the complex of the antibody of step a) with the candidate ligand bound thereto; and
f) identifying the ligand bound to the antibodies of step a).

43. A method for the diagnosis of acute coronary syndrome (ACS) in a patient comprising the step of contacting a biological sample from a patient selected from the group consisting of whole blood, serum and atherosclerotic coronary plaque with the antibody—or any fragment thereof—according to claim 2.

44. The diagnostic method of claim 43 for screening the population at risk of acute coronary syndrome (ACS).
